(19) United States
(12) Patent Application Publication
(10) Pub. No.: US 2013/0022551 A1
(43) Pub. Date: Jan. 24, 2013

(54) DEspR ANTAGONISTS AND AGONISTS AS THERAPEUTICS

(71) Applicants: Nelson Ruiz-Opazo, Westwood, MA (US); Victoria L.M. Herrera, Westwood, MA (US)

(72) Inventors: Nelson Ruiz-Opazo, Westwood, MA (US); Victoria L.M. Herrera, Westwood, MA (US)

(73) Assignee: TRUSTEES OF BOSTON UNIVERSITY, Boston, MA (US)

(21) Appl. No.: 13/356,233

(22) Filed: Jan. 23, 2012

Related U.S. Application Data
(63) Continuation-in-part of application No. PCT/US11/45056, filed on Jul. 22, 2011.

(51) Int. Cl. A61K 39/395; A61K 49/22; A61P 35/00; A61P 9/10; A61P 25/28; A61P 27/02; A61P 19/10; A61P 19/02; A61P 19/04; A61P 7/04; A61P 25/00; A61P 17/06; C07K 17/00; A61K 9/50; A61K 9/14; A61K 38/18

(52) U.S. Cl. 424/9.52; 424/130.1; 424/134.1; 514/8.1; 424/178.1; 424/142.1; 530/402; 424/490; 424/489

(57) ABSTRACT

Provided herein are novel compositions comprising DEspR-specific antagonists and agonists, and methods of their use in a variety of therapeutic applications. The compositions comprising the DEspR-specific anatgonists and agonists described herein are useful in therapeutic, diagnostic and imaging methods, such as DEspR-targeted molecular imaging of angiogenesis, and for companion diagnostic and/or in vivo-non invasive imaging and/or assessments.

### CROSS-REFERENCE TO RELATED APPLICATIONS

This application claims benefit under 35 U.S.C. §120 of PCT International Application Serial No.: PCT/US2011/45056 filed on 23 Jul. 2011, the contents of which are herein incorporated by reference in its entirety.

### GOVERNMENT SUPPORT

This invention was made with Government Support under Contract Nos.: NIH UL1 RR025771 and RO1 AG032649-01 awarded by the National Institutes of Health. The Government has certain rights in the invention.

### FIELD OF THE INVENTION

This invention relates to treatment of a variety of medical conditions, including for example stroke, adverse neurological events, cancer, cancer reoccurrence, and pathological angiogenesis. The invention further relates to compositions of matter and pharmaceutical compositions for treating medical conditions, including for example DEspR inhibitors, DEspR agonists, DEspR antagonists, DEspR agonists coupled to toxins, and antibodies to VEGFsp.

### BACKGROUND

The establishment of a critical role of the angiogenic switch in tumorigenesis has made the rationale behind the development of anti-angiogenesis therapy clear (Hanahan & Weinberg 2007). Unfortunately, the ability to attain long-term efficacy of anti-angiogenesis therapy for all cancer-types, in order to reduce cancer to a dormant, chronic manageable disease without increasing morbidity from side effects, has not yet been achieved (Loges et al. 2010, Ferrara 2009, Abdollahi & Folkman 2009, Bergers & Hanahan 2008).

Cumulative observations indicate that all three FDA-approved VEGF pathway inhibitors (anti-VEGFbevacizumab or Avastin, AntiVEGFR2 sunitinib, and sorafanib) result in only transitory improvements in the form of tumor stasis or shrinkage, and only for certain cancers despite most, if not all cancer types exhibiting pathological angiogenesis (Carmeliet 2005; Bergers and Hanahan 2008). Moreover, while anti-VEGF pathway therapies have reduced primary tumor growth and metastasis in preclinical studies (Crawford & Ferrara 2008), recent mouse tumor model studies have reported that sunitinib and an anti-VEGFR2 antibody, DC101, increased metastasis of tumor cells despite inhibition of primary tumor growth and increased overall survival in some cases (Ebos et al. 2009, Paez-Ribes et al. 2009). Addressing this “antiangiogenesis therapy conundrum,” cumulative observations have suggested several mechanisms of evasive and intrinsic resistances (Loges et al. 2010, Ferrara 2009, Abdollahi & Folkman 2009, Bergers & Hanahan 2008) such as: a) activation and/or upregulation of alternative pro angiogenic pathways, b) recruitment of bone marrow-derived pro-angiogenic cells, c) increased pericyte coverage for the tumor vasculature, attenuating the need for VEGF signaling; d) activation and enhancement of invasion and metastasis to provide access to normal tissue vasculature without obligate neovascularization; [for intrinsic resistance]: e) pre-existing multiplicity of redundant pro-angiogenic signals; f) pre-existing inflammatory cell-mediated vascular protection; g) tumor hypovascularity; and h) invasive and metastatic co-option of normal vessels without requisite angiogenesis (Bergers and Hanahan 2008).

Stroke remains a serious health problem affecting millions annually. For stroke survivors, the issue becomes ameliorating the potentially debilitating effects of the stroke. Finding therapies to ameliorate the effects of stroke remains a critical medical goal.

### SUMMARY OF THE INVENTION

This invention relates to treatment of a variety of medical conditions. The invention further relates to compositions of matter and pharmaceutical compositions for treating medical conditions. The invention further relates to DEspR inhibitors, DEspR agonists, DEspR antagonists, DEspR agonists coupled to toxins, and antibodies to VEGFsp. Described herein are novel compositions comprising DEspR agonists coupled to toxins, including DEspR agonists that are antibodies to DEspR (and DEspR binding portions thereof) coupled to toxin and VEGFsp (and DEspR binding portions thereof) coupled to toxin. Also described herein are anti-VEGFsignal peptide (anti-VEGFsp) antibodies and fragments thereof that bind VEGFsp, including human and humanized, monoclonal and polyclonal antibodies and fragments thereof, and VEGFsp fusion proteins. Also described herein are pharmaceutical preparations containing DEspR inhibitors, DEspR agonists, DEspR antagonists, DEspR agonists coupled to toxins, and antibodies to VEGFsp in methods of use in a variety of applications, including, but not limited to: 1) anti-angiogenesis therapies, treating stroke; inhibiting adverse neurological events, treating cancer, preventing cancer reoccurrence, and anti-angiogenesis approaches relevant to treatment of those vascular diseases where pathological angiogenesis plays a role in pathogenesis or progression, such as in age-related macular degeneration, carotid artery disease, diabetic retinopathy, rheumatoid arthritis, a neurodegenerative disease, Alzheimer's disease, obesity, endometriosis, psoriasis, atherosclerosis, ocular neovascularization, neovascular glaucoma, osteoporosis, or restenosis. Also provided herein are compositions and methods of using anti-DEspR antibodies and fragments thereof, including human and humanized, monoclonal and polyclonal anti-DEspR antibodies and fragments thereof to treat stroke and adverse neurological events such as micro-hemorrhages, recurrent cerebral hemorrhage and neurological deficit.

In some aspects, provided herein are methods of treating a subject having a stroke, the method comprises administering to a human subject within two days of the subject having a stroke a DEspR inhibitor in an amount effective to treat the stroke. The stroke may be an ischemic stroke, or a hemorrhagic stroke. The DEspR inhibitor may be administered to the subject within two days, within one day, within 12 hours, within 4 hours, within 2 hours, or within an hour of the subject having the stroke.

In some embodiments, the DEspR inhibitor comprises a monoclonal antibody or fragment thereof that binds DEspR or VEGFsp. In some embodiments, the DEspR inhibitor comprises a human or humanized monoclonal antibody or fragment that binds DEspR or VEGFsp. The human or humanized monoclonal antibody or fragment thereof, in some embodiments, binds to residues 1-9 of SEQ ID NO. 1. In some embodiments, the human or humanized monoclonal antibody comprises (i) a heavy chain variable region that is SEQ ID No. 4, (ii) a light chain variable region that is SEQ ID No. 9, or a heavy chain variable region that is SEQ ID No. 4 and a light chain variable region that is SEQ ID No. 9. In some embodiments, the human or humanized monoclonal antibody or fragment thereof binds VEGFsp. In some embodiments, the human antibody is a composite antibody.

In some aspects, provided herein are compositions comprising a DEspR inhibitor for treating stroke. In some embodiments, the DEspR inhibitor comprises a monoclonal antibody or fragment thereof that binds DEspR or VEGFsp. In some embodiments, the DEspR inhibitor comprises a human or humanized monoclonal antibody or fragment thereof that binds DEspR or VEGFsp. The human or humanized monoclonal antibody or fragment thereof, in some embodiments, binds to residues 1-9 of SEQ ID NO. 1. In some embodiments, the human or humanized monoclonal antibody comprises (i) a heavy chain variable region that is SEQ ID No. 4, (ii) a light chain variable region that is SEQ ID No. 9, or a heavy chain variable region that is SEQ ID No. 4 and a light chain variable region that is SEQ ID No. 9. In some embodiments, the human or humanized monoclonal antibody or fragment thereof binds VEGFsp of SEQ IF NO: 2. In some embodiments, the human antibody is a composite antibody.

In some aspects, provided herein are methods of inhibiting an adverse neurological event. The method comprises administering to a human subject having or suspected of having micro-hemorrhages a DEspR inhibitor in an amount effective to inhibit the adverse neurological event. The adverse neurological event, in some embodiments is further micro-hemorrhages, recurrent cerebral hemorrhage and/or neurological deficit. In some embodiments, the presence of micro-hemorrhages in the subject is detected prior to treatment with the DEspR inhibitor. In some embodiments, the subject is suspected of or at risk a known risk of having micro-hemorrhages prior to treatment with the DEspR inhibitor.

In some embodiments, the DEspR inhibitor comprises a monoclonal antibody or fragment thereof that binds DEspR or VEGFsp. In some embodiments, the DEspR inhibitor comprises a human or humanized monoclonal antibody or fragment that binds DEspR or VEGFsp. The human or humanized monoclonal antibody or fragment thereof, in some embodiments, binds to residues 1-9 of SEQ ID NO. 1. In some embodiments, the human or humanized monoclonal antibody comprises (i) a heavy chain variable region that is SEQ ID No. 4, (ii) a light chain variable region that is SEQ ID No. 9, or a heavy chain variable region that is SEQ ID No. 4 and a light chain variable region that is SEQ ID No. 9. In some embodiments, the human or humanized monoclonal antibody or fragment thereof binds VEGFsp. In some embodiments, the human antibody is a composite antibody.

In some aspects, provided herein are compositions for inhibiting an adverse neurological event comprising a DEspR inhibitor. In some embodiments, the DEspR inhibitor comprises a monoclonal antibody or fragment thereof that binds DEspR or VEGFsp. In some embodiments, the DEspR inhibitor comprises a human or humanized monoclonal antibody or fragment thereof that binds DEspR or VEGFsp. The human or humanized monoclonal antibody or fragment thereof, in some embodiments, binds to residues 1-9 of SEQ ID NO. 1. In some embodiments, the human or humanized monoclonal antibody comprises (i) a heavy chain variable region that is SEQ ID No. 4, (ii) a light chain variable region that is SEQ ID No. 9, or a heavy chain variable region that is SEQ ID No. 4 and a light chain variable region that is SEQ ID No. 9. In some embodiments, the human or humanized monoclonal antibody or fragment thereof binds VEGFsp. In some embodiments, the human antibody is a composite antibody.

In some aspects, provided herein are methods of treating cancer. The method comprises administering to a subject having a cancer expressing DEspR an antibody or fragment thereof that binds selectively to VEGFsp in an amount effective to inhibit the cancer. The antibody or fragment thereof may be a monoclonal antibody or a humanized monoclonal antibody. The antibody or fragment thereof may block the binding of VEGFsp to DEspR. In some embodiments, the antibody has an Fc region modified to promote clearance from circulation of the antibody.

In some aspects, provided herein are methods of inhibiting angiogenesis. The method comprises administering to a subject having a disease or disorder dependent on or modulated by angiogenesis, an antibody or fragment thereof that binds selectively VEGFsp in an amount effective to inhibit the angiogenesis. In some embodiments, the disease or disorder is cancer, age-related macular degeneration, carotid artery disease, diabetic retinopathy, rheumatoid arthritis, a neurodegenerative disease, Alzheimer's disease, obesity, endometriosis, psoriasis, atherosclerosis, ocular neovascularization, neovascular glaucoma, osteoporosis, or restenosis. The antibody or fragment thereof may be a monoclonal antibody or a human or humanized monoclonal antibody. In some embodiments, the human antibody is a composite antibody. The antibody or fragment thereof may block the binding of VEGFsp to DEspR. In some embodiments, the antibody has an Fc region modified to promote clearance from circulation of the antibody.

In some aspects, provided herein are pharmaceutical preparations comprising a human or humanized antibody or fragment thereof that binds selectively VEGFsp and a pharmaceutically acceptable carrier constructed and arranged for administration to a human. In some embodiments, the antibody or fragment thereof is a monoclonal antibody. In some embodiments, the human antibody is a composite antibody. The antibody or fragment thereof may block binding of VEGFsp to DEspR. In some embodiments, the pharmaceutical preparation comprises the antibody. In some embodiments, the pharmaceutical preparation comprises the fragment. In some embodiments, the antibody has an Fc region modified to promote clearance from circulation of the antibody.

In some aspects, provided herein are compositions comprising a DEspR agonist coupled to a toxin. In some embodiments the agonist is an antibody or fragment thereof that binds selectively DEspR. In some embodiments the agonist is VEGFsp or a fragment thereof that binds DEspR. The agonist, such as the antibody or fragment thereof that selectively binds DEspR or the VEGFsp or fragment thereof that binds DEspR, is coupled to a toxin. The agonist may be covalently or non-covalently coupled to a toxin. In some embodiments, the DEspR agonist that binds DEspR is coupled to a particle that is coupled to, coated with, embedded with or contains the toxin. In some embodiments, the particle is a solid polymer matrix or a liposome. The toxin may be a radiotoxin or a chemotoxin. In any of the foregoing embodiments the agonist is VEGFsp or a fragment thereof that binds DEspR.

In some aspects, provided herein are pharmaceutical preparations comprising VEGFsp, or a fragment thereof that binds DEspR, coupled to a toxin and a pharmaceutically acceptable carrier constructed and arranged for administration to a human. The VEGFsp or fragment thereof may be covalently or non-covalently coupled to a toxin. In some embodiments, the VEGFsp or the fragment thereof that binds DEspR is coupled to a particle that is coupled to, coated with, embedded with or contains the toxin. In some embodiments, the particle is a solid polymer matrix or a liposome. The toxin may be a radiotoxin or a chemotoxin.

In some aspects, provided herein are pharmaceutical preparations comprising an antibody or fragment thereof that binds selectively DEspR, coupled to a toxin and a pharmaceutically acceptable carrier constructed and arranged for administration to a human. The antibody or fragment thereof that binds selectively DEspR may be covalently or non-covalently coupled to a toxin. In some embodiments, the antibody or fragment thereof that binds selectively DEspR is coupled to a particle that is coupled to, coated with, embedded with or contains the toxin. In some embodiments, the particle is a solid polymer matrix or a liposome. The toxin may be a radiotoxin or a chemotoxin.

In some aspects, provided herein are methods for inhibiting growth of tumor cells. The method comprises contacting tumor cells expressing DEspR with a DEspR agonist coupled to a toxin in an amount effective to inhibit growth of the tumor. In some embodiments, the tumor cells are in a subject who has had one or more of (i) radiation treatment for cancer, (ii) chemotherapy for cancer, or (iii) surgical treatment for cancer. In some embodiments, the DEspR-agonist is an antibody or fragment thereof that binds DEspR or is VEGFsp or a fragment thereof that binds DEspR. In some embodiments, the antibody or fragment thereof that binds DEspR is a monoclonal antibody. In some embodiments, the antibody or fragment thereof that binds DEspR is a human or humanized monoclonal antibody. The DEspR agonist may be covalently or non-covalently coupled to the toxin. In some embodiments, the DEspR agonist is coupled to a particle that is coupled to, coated with, embedded with or contains the toxin. In some embodiments, the particle is a solid polymer matrix or a liposome. The toxin may be a radiotoxin or a chemotoxin. In some embodiments, inhibiting growth of tumor cells means that the tumor size is halted from growing or is reduced. In some embodiments, tumor metastasis is inhibited. In some embodiments, side effects or complications associated with the cancer are reduced or progression of such tumor side effects or complications are inhibited or halted. In some aspects, provided herein are methods of reducing cancer re-occurrence. The method comprises administering to a subject after the subject has had one or more of (i) radiation treatment for cancer, (ii) surgical treatment for cancer and (iii) chemotherapy treatment for cancer, a DEspR inhibitor in an amount effective to reduce cancer re-occurrence. In some embodiments, the DEspR inhibitor comprises an antibody or fragment thereof that binds DEspR or VEGFsp as described herein.

In some aspects, provided herein is a method for identifying a circulating tumor cell comprising contacting a circulating tumor cell expressing DEspR with an agent that binds DEspR, and detecting the agent bound to the circulating tumor cell. In some embodiments, the agent is (i) an antibody that binds DEspR or (ii) VEGFsp. In some embodiments, the agent is labeled.

In addition, compositions comprising VEGFsp coupled to a label or to an imaging moiety are useful in diagnostic and imaging methods, such as DEspR-targeted molecular imaging of angiogenesis, which can be used, for example, in monitoring response to therapy, in vivo detection of tumor “angiogenic switch” or vascular mimicry. The compositions comprising the anti-DEspR antibodies and fragments thereof are useful for novel companion diagnostic and/or in vivo-non invasive imaging and/or assessments. Additionally, VEGFsp may be coupled directly or indirectly to agents other than a label or a toxin, to deliver such agent to a tissue expressing DEspR. Accordingly, the compositions comprising the VEGFsp and fragments thereof that bind DEspR, coupled to an agent, comprise targeting tools and/or modules for target-specific delivery of therapeutics, in forms such as toxins, drugs, small molecules, peptides, fusion proteins, chimeric proteins, carriers such as nanoparticles and liposomes, DNA, siRNA, etc., as well as for combinatorial target-specific diagnostics and therapeutics, termed herein as “theragnostics.”

In some embodiments of these aspects and all such aspects described herein, the anti-DEspR antibody or antibody fragment thereof specifically binds to DEspR comprising the amino acid sequence of SEQ ID NO: 1. In some embodiments of these aspects, the antibody or antibody fragment thereof specifically binds to an epitope of DEspR comprising some or all of residues 1-9 of SEQ ID NO: 1. In some embodiments of these aspects, the antibody or antibody fragment thereof specifically binds to an epitope of DEspR consisting essentially of residues 1-9 of SEQ ID NO: 1. In some embodiments of these aspects, the antibody or antibody fragment thereof specifically binds to an epitope of DEspR consisting of residues 1-9 of SEQ ID NO: 1. In some embodiments of these aspects, the antibody or antibody fragment thereof was generated using a portion of DEspR that consists of residues 1-9 of SEQ ID NO: 1.

In some embodiments of these aspects and all such aspects described herein, the anti-DEspR antibody or antibody fragment thereof specifically binds to DEspR at a VEGF signal peptide (VEGFsp) binding site.

In some embodiments, the VEGF signal peptide comprises the amino acid sequence of SEQ ID NO: 2. In some embodiments, the VEGF signal peptide consists essentially of the amino acid sequence of SEQ ID NO: 2. In some embodiments, the VEGF signal peptide consists of the amino acid sequence of SEQ ID NO: 2.

In some embodiments of these aspects and all such aspects described herein, the anti-DEspR antibody is a monoclonal antibody or antibody fragment thereof. In some embodiments of these aspects and all such aspects described herein, the anti-DEspR antibody is a human or humanized antibody or antibody fragment thereof. In some embodiments, the human antibody is a composite antibody.

In some embodiments of these aspects and all such aspects described herein, the anti-DEspR antibody or antibody fragment comprises one or more heavy chain CDR regions comprising a sequence selected from the group consisting of SEQ ID NO: 5, SEQ ID NO: 6, and SEQ ID NO: 7. In some such embodiments, one or more heavy chain CDR regions consist essentially of a sequence selected from the group consisting of SEQ ID NO: 5, SEQ ID NO: 6, and SEQ ID NO: 7. In some such embodiments, one or more heavy chain CDR regions consist of a sequence selected from the group consisting of SEQ ID NO: 5, SEQ ID NO: 6, and SEQ ID NO: 7.

In some embodiments of these aspects and all such aspects described herein, the anti-DEspR antibody or antibody fragment comprises one or more light chain CDR regions comprising a sequence selected from the group consisting of SEQ ID NO: 10, SEQ ID NO: 11, and SEQ ID NO: 12. In some such embodiments, one or more light chain CDR regions consist essentially of a sequence selected from the group consisting of SEQ ID NO: 10, SEQ ID NO: 11, and SEQ ID NO: 12. In some such embodiments, one or more light chain CDR regions consist of a sequence selected from the group consisting of SEQ ID NO: 10, SEQ ID NO: 11, and SEQ ID NO: 12.

In some embodiments of these aspects and all such aspects described herein, the anti-DEspR antibody or antibody fragment comprises one or more heavy chain CDR regions comprising a sequence selected from the group consisting of SEQ ID NO: 5, SEQ ID NO: 6, and SEQ ID NO: 7, and one or more light chain CDR regions comprising a sequence selected from the group consisting of SEQ ID NO: 10, SEQ ID NO: 11, and SEQ ID NO: 12. In some such embodiments, the one or more heavy chain CDR regions consist essentially of a sequence selected from the group consisting of SEQ ID NO: 5, SEQ ID NO: 6, and SEQ ID NO: 7. In some such embodiments, the one or more heavy chain CDR regions consist of a sequence selected from the group consisting of SEQ ID NO: 5, SEQ ID NO: 6, and SEQ ID NO: 7. In some such embodiments, the one or more light chain CDR regions consist essentially of a sequence selected from the group consisting of SEQ ID NO: 10, SEQ ID NO: 11, and SEQ ID NO: 12. In some such embodiments, the one or more light chain CDR regions consist of a sequence selected from the group consisting of SEQ ID NO: 10, SEQ ID NO: 11, and SEQ ID NO: 12.

In some embodiments of these aspects and all such aspects described herein, the anti-DEspR antibody is a composite antibody or antibody fragment thereof. In some such embodiments, the anti-DEspR composite antibody or antibody fragment comprises one or more heavy chain CDR regions comprising a sequence selected from the group consisting of SEQ ID NO: 5, SEQ ID NO: 6, and SEQ ID NO: 7. In some such embodiments, the anti-DEspR composite antibody or antibody fragment comprises one or more heavy chain CDR regions consisting essentially of a sequence selected from the group consisting of SEQ ID NO: 5, SEQ ID NO: 6, and SEQ ID NO: 7. In some such embodiments, the anti-DEspR composite antibody or antibody fragment comprises one or more heavy chain CDR regions consisting of a sequence selected from the group consisting of SEQ ID NO: 5, SEQ ID NO: 6, and SEQ ID NO: 7. In some such embodiments, the anti-DEspR composite antibody or antibody fragment comprises one or more light chain CDR regions comprising a sequence selected from the group consisting of SEQ ID NO: 10, SEQ ID NO: 11, and SEQ ID NO: 12. In some such embodiments, the anti-DEspR composite antibody or antibody fragment comprises one or more light chain CDR regions consisting essentially of a sequence selected from the group consisting of SEQ ID NO: 10, SEQ ID NO: 11, and SEQ ID NO: 12. In some such embodiments, the anti-DEspR composite antibody or antibody fragment comprises one or more light chain CDR regions consists of a sequence selected from the group consisting of SEQ ID NO: 10, SEQ ID NO: 11, and SEQ ID NO: 12.

In some embodiments of these aspects and all such aspects described herein, the anti-DEspR antibody is a composite antibody or antibody fragment thereof comprising a variable heavy (VH) chain amino acid sequence selected from the group consisting of SEQ ID NO: 13-SEQ ID NO: 17. In some embodiments of these aspects and all such aspects described herein, the anti-DEspR antibody is a composite antibody or antibody fragment thereof consisting essentially of a variable heavy (VH) chain amino acid sequence selected from the group consisting of SEQ ID NO: 13-SEQ ID NO: 17. In some embodiments of these aspects and all such aspects described herein, the anti-DEspR antibody is a composite antibody or antibody fragment thereof consisting of a variable heavy (VH) chain amino acid sequence selected from the group consisting of SEQ ID NO: 13-SEQ ID NO: 17.

In some embodiments of these aspects and all such aspects described herein, the anti-DEspR antibody is a composite antibody or antibody fragment thereof comprising a variable light (VL) chain amino acid sequence selected from the group consisting of SEQ ID NO: 18 and SEQ ID NO: 19. In some embodiments of these aspects and all such aspects described herein, the anti-DEspR antibody is a composite antibody or antibody fragment thereof consisting essentially of a variable light (VL) chain amino acid sequence selected from the group consisting of SEQ ID NO: 18 and SEQ ID NO: 19. In some embodiments of these aspects and all such aspects described herein, the anti-DEspR antibody is a composite antibody or antibody fragment thereof consisting of a variable light (VL) chain amino acid sequence selected from the group consisting of SEQ ID NO: 18 and SEQ ID NO: 19.

In other embodiments of these aspects, the anti-DEspR antibody or antibody fragment thereof is an antibody expressed or produced by hybridomas 7C5C5 or 5G12E8. In some aspects, provided herein are methods of inhibiting tumor cell invasiveness in a subject having a cancer or a tumor, comprising administering to a subject in need thereof a therapeutically effective amount of a pharmaceutical composition comprising any of the anti-VEGFsp antibodies or antibody fragments thereof or DEspR agonists coupled to toxin, described herein. In some embodiments of these aspects and all such aspects described herein, the method further comprises the administration of one or more chemotherapeutic agents, angiogenesis inhibitors, cytotoxic agents, or anti-proliferative agents.

In some aspects, provided herein are methods of inhibiting tumor growth and reducing tumor size or tumor metastasis in a subject having a tumor or metastasis by inhibiting DEspR expression and/or function in a cell, comprising administering to a subject in need thereof a therapeutically effective amount of a pharmaceutical composition comprising any of the anti-VEGFsp antibodies or antibody fragments thereof or DEspR agonists coupled to toxin, described herein. In some embodiments of these aspects, the DEspR expression and/or function is inhibited in a tumor cell, a tumor initiating cell, a cancer stem-like cell, a cancer stem cell, a metastatic tumor cell, a circulating tumor cell, an endothelial progenitor cell, an inflammatory cell, a tumor stromal cell, a tumor vasculature cell, or any combination thereof. In some such embodiments, the tumor vasculature cell is an endothelial cell, a pericyte, a smooth muscle cell, an adventitial cell, or any combination thereof. In some embodiments of these aspects, the toxin kills a tumor cell, a tumor initiating cell, a cancer stem-like cell, a cancer stem cell, a metastatic tumor cell, a circulating tumor cell, an endothelial progenitor cell, an inflammatory cell, a tumor stromal cell, a tumor vasculature cell, or any combination thereof. In some such embodiments, the tumor vasculature cell is an endothelial cell, a pericyte, a smooth muscle cell, an adventitial cell, or any combination thereof.

In some aspects, provided herein are methods for enhancing delivery of a therapeutic agent via DEspR-targeted sonoporation, the methods comprising delivering an effective amount of a pharmaceutical composition comprising VEGFsp or a DEspR binding fragment of VEGFsp, as the targeting moiety, and a therapeutic agent using targeted ultrasound delivery, to a subject in need thereof, such that delivery of the therapeutic agent is enhanced or increased relative to delivering the therapeutic agent in the absence of the pharmaceutical composition comprising VEGFsp or a DEspR binding fragment of VEGFsp. In some embodiments of these aspects and all such aspects described herein, the therapeutic agent is a chemotherapeutic agent, a small molecule, a peptide, or an aptamer.

In some aspects, provided herein are pharmaceutical compositions comprising any of the VEGFsp or a DEspR binding fragment of VEGFsp for use in enhancing delivery of a therapeutic agent via DEspR-targeted sonoporation using targeted ultrasound delivery to a subject in need thereof. In some embodiments of these aspects and all such aspects described herein, the therapeutic agent is a chemotherapeutic agent, a small molecule, a peptide, or an aptamer.

In some aspects, provided herein are pharmaceutical compositions comprising any of the VEGFsp or a DEspR binding fragment of VEGFsp for use in reducing toxicity of a therapeutic agent via DEspR-targeted sonoporation using targeted ultrasound delivery to a subject in need thereof. In some embodiments of these aspects and all such aspects described herein, the therapeutic agent is a chemotherapeutic agent, a small molecule, a peptide, or an aptamer.

### BRIEF DESCRIPTION OF THE FIGURES

FIGS. 1A-1E show that inhibition of angiogenesis neovessel tube length is seen using both anti-DEspR (Ab1) and anti-VEGFsp (Ab2) antibodies in HUVECs (FIG. 1D) and HMECs (FIG. 1E) angiogenesis assays. (Tukey's all pairwise multiple comparison P <0.001 for both HUVECs and HMECs). Similar findings were observed for other angiogenesis parameters including neovessel branching and inter-connections made.

FIGS. 2A-2B show that in contrast to control (C) and pre-immune ab treatment (PI), DEspR-inhibition via anti-human DEspR antibody treatment inhibits tumor cell invasiveness in two cell lines tested, metastatic breast tumor MDA-MB-468 and pancreatic adenocarcinoma PANC-1 cell lines.

FIG. 3 shows that anti-DEspR treated rats (□) exhibited minimal tumor growth compared with mock-treated controls (▪), two-tailed t-test *P <0.05; **P <0.001.

FIG. 4 shows characterization of selected monoclonal antibodies. Monoclonal antibodies 2E4A8, 2E4B11, 2E4H10, 5G12E8, 7C5B2, 7C5C5, 8E7D11, 8E2F6, E2G4 and 8E7F8 were tested by indirect ELISA using standard procedures. Serial dilutions from supernatants containing monoclonal antibodies at 1 μg/ml were tested as follows: 1=1/2; 2=1/4; 3=1/8; 4=1/16; 5=1/32; 6=1/64; 7=1/128; 8=1/256; 9=1/512; 10=1/1024; 11=1/2048 and 12=1/4096.

FIG. 5 shows Western blot analysis of monoclonal antibodies tested. To ascertain specificity, low- (5G12E8), mid- (2E4H6), and high-affinity (7C5B2) monoclonal antibodies were tested as well as the subclone supernatant, and the subsequent purified antibody. The anti-human DEspR monoclonal antibodies are specific for the predicted 10 kD protein for human DEspR. Western blot analysis was performed using total cellular protein isolated from Cos1 human DEspR-transfected cells as antigen, primary antibody comprised purified antibody and subclone supernatant of 3 selected clones, 10% gel concentration in order to detect the expected 10 kD molecular weight protein of human DEspR. Nitrocellulose (PIERCE) with a transfer buffer of 3.07 g Tris, 14.4 g Glycine, 200 ml methanol, 800 ml dH₂O were used. HRP-anti mouse polyvalent immunoglobulins (Sigma #0412) were used at 1:100,000; ECL reagent (SuperSignal West Femto Kit #34094), Stain reagent Kodak RP-X-Omat, and x-film (Kodak X-film #XBT-1). The Western blot results demonstrate specificity of anti-human DEspR monoclonal antibodies regardless of relative affinity, thus identifying more than one successful anti-human DEspR monoclonal antibody. The results indicate that the monoclonal antibody clone with highest relative affinity and specificity is clone 7C5B2.

FIGS. 6A-6C show inhibition of different parameters of angiogenesis by monoclonal antibody 7C5B2 and a polyclonal antibody preparation to DEspR. 7C5B2 monoclonal antibody was shown to immunostain HUVECs undergoing tube formation, pancreatic adenocarcinoma PANC-1, and triple negative breast cancer MDA-MB-231 and -468 cells. FIG. 6A shows mean number of branch points as a measure of neovessel complexity, and total length of tubes as a measure of neovessel density is shown in FIG. 6B. FIG. 6C shows concentration-dependent inhibition of in vitro serum-induced HUVEC tubulogenesis by monoclonal antibody 7C5B2. HUVEC (human umbilical vein endothelial cells) were grown onto Matrigel-coated wells in basal medium supplemented with 2% FBS (control), or 2% FBS+monoclonal antibody 7C5B2 (0.05-500 nM). The percentage of serum-induced tubulogenesis was determined as the difference between HUVECs grown in control conditions and the indicated monoclonal antibody 7C5B2-supplemented media. The % of the total tube length per well and the total number of branching points per well in the in vitro tube formation assay is presented. Data are shown as mean±standard error. Each experimental condition was performed in five replica wells. EC₅₀ for total tube length=4.34±0.45 nM; EC₅₀ for # branching points=3.97±0.51 nM.

FIGS. 7A-7C demonstrate that a monoclonal antibody 7C5B2 inhibits tumor cell invasiveness in MDA-MB-468 human breast cancer (FIG. 7A) and PANC-1 pancreatic cancer (FIG. 7B) cell lines (P <0.001*, <0.01*). FIG. 7C shows dose response curve of inhibition of MDA-MB-468 cell invasion by monoclonal antibody 7C5B2 (EC₅₀=3.55±0.32 nM). Data, mean±standard error of 5 replicates. *P <0.001,**P <0.01 (one way ANOVA, all pairwise multiple comparison Tukey's Test).

FIGS. 8A-8D show effects of an anti-human DEspR monoclonal antibody 7C5B2 (IgG2b isotype) on in vitro serum-induced HUVEC tubulogenesis (established in vitro angiogenesis assay). HUVECs (human umbilical vein endothelial cells) were grown onto Matrigel-coated wells in basal medium supplemented with 2% FBS (control C1), or 2% FBS+pre-immune IgG isotype control for polyclonal anti-hDESPR antibody (500 nM, control C2), or 2% FBS+IgG2b isotype control for anti-hDESPR mAB (500 nM, C3 control) or 2% FBS+polyclonal anti-hDEspR (500 nM, P) or 2% FBS+monoclonal antibody 7C5B2 (500 nM, M). Quantitative analysis of the mean number of tubes formed per well is shown in FIG. 8A, the mean number of branching points per well is shown in FIG. 8B, the mean number of connections per well is shown in FIG. 8C and the mean total tube length in mm per well is shown in FIG. 8D, using the in vitro tube formation assay. Data are shown as mean±standard error. Each experimental condition was performed in five replica wells. Statistically significant differences (as compared with respective control conditions), are indicated as follows: *P <0.001 (one way ANOVA followed by all pairwise multiple comparison Tukey Test).

FIGS. 9A-9D depict quantitation of contrast intensity done using integrated VisualSonics Micro-imaging System software (FIG. 9D) and demonstrates increased contrast intensity in DEspR+carotid artery endothelium and vasa vasorum, in contrast to both low contrast intensity in DEspR(−) endothelium and vasa vasorum, and isotype-microbubble controls. P <0.0001, ANOVA and pairwise multiple comparison. Anti-DEspR-antibody is biotinylated and coupled to streptavidin-PEG coated commercially available microbubbles for ultrasound analysis and imaging.

FIGS. 10A-10F show immunohistochemical analysis of DEspR expression in human breast tissue using an anti-DEspR monoclonal antibody (FIGS. 10A-10) normal; Grade-1, T1 invasive ductal carcinoma (FIGS. 10D-10F). FIG. 10A shows normal breast tissue: 3×-overlay of DEspR, aSMA and DAPI nuclear stain detects aSMA expression in mammary myoepithelial cells but no expression of DEspR in epithelial cells and microvessels. FIG. 10B shows 2×-immunofluorescence overlay of DEspR and DAPI nuclear stain and confirms absence of DEspR expression in normal breast tissue. FIG. 10C is a 4×-overlay of DEspR, aSMA, DAPI immunofluorescence and diffusion contrast imaging (DIC) that delineates tissue morphology, expression of aSMA and non/minimal-expression of DEspR in normal mammary epithelium and endothelium. FIG. 10D is a 3×-Overlay of DAPI, aSMA and DEspR immunofluorescence in Gr.I-T1 invasive ductal carcinoma that detects DEspR expression in vascular endothelium, and co-localization with aSMA in mammary tissue. FIG. 10E is a 2×-overlay of DAPI and DEspR of breast cancer shown in panel 17D that highlights DEspR expression. FIG. 10F is a 4×-overlay of DAPI, aSMA, DEspR, DIC to elucidate DEspR spatial expression with tissue morphology of epithelial cells and microvessels. bar=20 microns.

FIGS. 11A-11F show monoclonal antibody immunohistochemical analysis of DEspR expression in normal pancreatic tissue (FIGS. 11A-11C) normal; and Grade-3, T3 pancreatic ductal carcinoma (FIGS. 11D-11F). FIG. 11A shows that normal pancreatic tissue, with a 3×-overlay of DEspR, aSMA and DAPI nuclear stain, detects minimal DEspR expression in microvessels. FIG. 11B shows a 4×-immunofluorescence overlay of DEspR, aSMA, DAPI, with DIC imaging of tissue morphology. FIG. 11C (left) shows a 3×-overlay of DEspR, aSMA, DAPI immunofluorescence; (right) shows a 4×-overlay of DEspR, aSMA, DAPI and diffusion contrast imaging (DIC) for tissue morphology that shows aSMA expression and non/minimal-expression of DEspR in normal endothelium. FIG. 11D shows that 3×-overlay of DAPI, aSMA and DEspR immunofluorescence in Gr.3-T3 pancreatic ductal carcinoma detects DEspR expression in vascular endothelium, and co-localization with aSMA. FIG. 11E shows a 2×-overlay of DAPI and DEspR of the image shown in FIG. 12A and highlights DEspR expression. FIG. 11F shows a 3×-overlay of DAPI, aSMA, DEspR, that shows increased DEspR expression in pancreatic ductal carcinoma cells. bar=20 microns.

FIGS. 12A-12B show representative contrast enhanced ultrasound (CEU)-images with contrast intensity signals (CIS) depicted. FIG. 12A shows a graph of CIS-differences (Δ) among different study groups as notated distinguishing CEU-positive imaging in Tg MB_{D} CEU+ group from the other CEU-negative groups. FIG. 12B shows a graph of CIS-difference between all transgenic rats (Tg+) and non-transgenic rats (nonTg). Hatched bar represents a threshold between MB_{D}-infused CEU+ and MB_{D}-infused CEU-transgenic rats. Blood pool, CEU-image 1 minute after bolus injection of MBs, demonstrating equivalent MB-infusion among different rats and minimal contrast-intensity signals from movement artifacts. 1-Pre, pre-acoustic destruction CEU-images obtained 4-minutes after bolus infusion, in order to allow MB-adherence to target, if any, and to document minimal, if any, circulating MBs in the lumen. Image corresponds to #1 on CIS-plot. 2-Post, CEU-image after acoustic destruction corresponding to #2 on scatter plot. CIS-plot, scatter plot of contrast-intensity signals (CIS) in representative regions of interest (encircled in aqua). #1, CIS detected pre-acoustic destruction; #2, CIS detected post-acoustic destruction (2). Black line and following gap mark period of acoustic destruction in CIS-scatter plots. MB_{D}, DEspR-targeted microbubble; MB_{C}, control isotype-targeted microbubble; Tg, transgenic rat; nonTg, nontransgenic control rat; CEU+, CEU positive imaging; CEU-, CEU negative imaging, Δ Contrast Intensity, pre-/post-destruction CIS-difference; ***, P <0.0001.

FIGS. 13A-13H depict representative MB_{D}-specific contrast enhanced ultrasound (CEU)-positive images depicting complex pattern of acoustic destruction of adherent MB_{D}-microbubbles in a transgenic rat, R3. FIG. 13A shows representative CEU-image documenting blood pool of circulating MB_{D}s filling carotid artery lumen one-minute after bolus infusion. CCA, common carotid artery; ECA, external carotid artery; ICA, internal carotid artery; *, CCA bifurcation. FIGS. 13B-13D show scatter plots of contrast-intensity signals marked with same-dashed blocks to refer to corresponding regions of interest (ROI) in panel-13E. (13B) white solid line; (13C), white hatched line; (13D) white dotted line ROIs. FIG. 13E shows representative CEU-image that corresponds to #1 on scatter plots b,c,d documenting adherent DEspR-targeted microbubbles (MB_{D}) just prior to pre-acoustic destruction (black line). Adherent MB_{D}s are seen in the three ROIs encircled white solid line, white hatched line, and white dotted line. FIG. 13F shows representative CEU-image corresponding to #2 on scatter plots b-d showing a post-acoustic destruction dip in signal intensity compared to levels in #1 in the different ROIs respectively. FIG. 13G shows representative CEU-image corresponding to #3 on scatter plots b-d showing a post-acoustic destruction secondary peak in contrast intensity signals in the different ROIs. FIG. 13H shows representative CEU-image corresponding to #4 on scatter plots documenting the decline in contrast-intensity signals approaching baseline levels observed in isotype control or MB_{D}-infused CEU-negative images and demonstrating low background CIS levels.

FIG. 14 depicts representative fluorescence immunostaining analysis of carotid arteries from rats exhibiting MB_{D}-specific CEU-positive imaging and CEU-negative imaging. FIG. 14 shows scatter dot plot of pre-destruction CIS-peak levels highlighting a threshold (hatched bar) between MB_{D}-specific CEU-positive (CEU+) and CEU-negative (CEU−) imaging.

FIGS. 15A-15G depict phase contrast-fluorescence microscopy analysis of anti-humanDEspR-targeted microbubbles (MB_{D}) binding to human endothelial cells, HUVECs, in vitro. Increasing DEspR-targeted microbubbles (MB_{D}) to cell ratio (15A) 8×, (15B) 80×, and (15C) 800×. (15D) Isotype control (MB_{C}) at 800×; (15E) non-targeted control MB_{O} at 800×. (15F) % of HUVECs with bound MBs (▪) and no MB binding (□). FIG. 15G shows number of MBs (mean+/−sem) per bound cell with increasing MB to cell ratio: MB_{D} compared with isotype control MB_{C} and control non-targeted MB_{O}. ***, ANOVA P <0.0001.

FIGS. 16A-16B show characterization of a human-specific anti-DEspR monoclonal antibody. (16A) Analysis by indirect ELISA of 10 candidate monoclonal antibody clones is shown. Serial dilutions from supernatants containing mAbs at 1 μg/ml were tested as follows: 1=1/2; 2=1/4; 3=1/8; 4=1/16; 5=1/32; 6=1/64; 7=1/128; 8=1/256; 9=1/512; 10=1/1024; 11=1/2048 and 12=1/4096. white diamond, selected Mab 7c5b2 clone, open symbols, all others. (16B) Western blot analysis of purified Mabs (lanes 1-3), and “super clone” supernatants (lanes 4-6), with PBS serving as control (lane 7) are depicted. Selected 7C5B2 Mab in lanes 1 and 4 (diamond). Double immunostaining of HUVECs with anti-DEspR Mab-immunostaining and anti-VEGFsp immunostaining was performed and colocalization of DEspR and VEGFsp determined.

FIGS. 17A-17C demonstrate that DEspR inhibition via monoclonal antibody decreases angiogenesis in in vitro HUVECs assay. DEspR immunostaining of HUVECs using anti-DEspR Mab was performed. (17A) Dose response curve to anti-DEspR Mab inhibition of angiogenesis measuring total tube length per well (◯) with EC₅₀=4.34+/−0.45 nM; and number of tube branch points () with EC.₅₀ 3.97+/−0.51 nM. (17B) Analysis of total tube length changes upon DEspR-inhibition via anti-DEspR polyclonal (Pab) and monoclonal (Mab) antibodies compared to control untreated cells (17C), pre-immune serum (PI) and IgG2b isotype (Iso) controls for Pab and Mab, respectively. (17C) Analysis of mean number (#) of branch points inhibited by Pab and Mab anti-DEspR ab-inhibition compared with controls (C, PI, Iso). Data expressed as mean+/−sem; 4 replicates; *, P <0.01 (ANOVA followed by all pairwise multiple comparison Tukey test).

FIGS. 18A-18C demonstrate that DEspR inhibition via monoclonal antibody decreases tumor cell invasiveness in vitro. DEspR-positive immunostaining of MDA-MB-468 breast cancer cells and PANC-1 pancreatic cancer cell line via anti-DEspR Mab was performed. (18A) Dose response curve to increasing DEspR-inhibition via anti-DEspR Mab of MDA-MB-468 breast cancer cell invasiveness (black), EC₅₀=3.55+/−0.32 nM. (18B-18C) Analysis of cell invasiveness inhibited by anti-DEspR Mab inhibition compared to control untreated cells, and IgG2b isotype control for MDA-MB-468 breast cancer cells, and PANC-1 pancreatic cell line. All data shown as mean+/−sem of 4 replicates; *, P <0.01; **, P <0.001 (1-way ANOVA followed by all pairwise multiple comparison Tukey Test).

FIG. 19 demonstrates 1% agarose gel separation of RT-PCR products of antibody obtained from the 7C5B2 hybridoma. Gel was stained with SYBR® Safe DNA gel stain (Invitrogen cat. no. 533102) and photographed over ultraviolet light. Size marker (L) is GeneRuler™ 1 Kb Plus (Fermentas cat. no. SM1331). RT-PCR was performed using degenerate primer pools for murine signal sequences with constant region primers for each of IgGVH, IgMVH, IgκVL and IgλVL.

FIG. 20 shows the variable heavy chain amino acid (SEQ ID NO: 4) and nucleotide (SEQ ID NO: 3) sequence of the 7C5B2 antibody. CDR definitions and protein sequence numbering according to Kabat.

FIG. 21 shows the variable light chain amino acid (SEQ ID NO: 9) and nucleotide (SEQ ID NO: 8) sequence of a composite 7C5B2 antibody. CDR definitions and protein sequence numbering according to Kabat.

FIG. 22 shows an exemplary variable heavy chain amino acid (SEQ ID NO: 13) and nucleotide sequence of a composite anti-DEspR 7C5B2 antibody generated using the methods described herein. CDR definitions and protein sequence numbering according to Kabat.

FIG. 23 shows an exemplary variable heavy chain amino acid (SEQ ID NO: 14) and nucleotide sequence of a composite anti-DEspR 7C5B2 antibody generated using the methods described herein. CDR definitions and protein sequence numbering according to Kabat.

FIG. 24 shows an exemplary variable heavy chain amino acid (SEQ ID NO: 15) and nucleotide sequence of a composite anti-DEspR 7C5B2 antibody generated using the methods described herein. CDR definitions and protein sequence numbering according to Kabat.

FIG. 25 shows an exemplary variable heavy chain amino acid (SEQ ID NO: 16) and nucleotide sequence of a composite anti-DEspR 7C5B2 antibody generated using the methods described herein. CDR definitions and protein sequence numbering according to Kabat.

FIG. 26 shows an exemplary variable heavy chain amino acid (SEQ ID NO: 17) and nucleotide sequence of a composite anti-DEspR 7C5B2 antibody generated using the methods described herein. CDR definitions and protein sequence numbering according to Kabat.

FIG. 27 shows an exemplary variable light chain amino acid (SEQ ID NO: 18) and nucleotide sequence of a composite anti-DEspR 7C5B2 antibody generated using the methods described herein. CDR definitions and protein sequence numbering according to Kabat.

FIG. 28 shows an exemplary variable light chain amino acid (SEQ ID NO: 19) and nucleotide sequence of a composite anti-DEspR 7C5B2 antibody generated using the methods described herein. CDR definitions and protein sequence numbering according to Kabat.

FIG. 29 shows the effect of anti-DEspR treatment on stroke survival in Tg25 stroke-prone Dahl S rat model (Dahl S rats transgenic for human cholesteryl ester transfer protein). Tg25 female rats were treated (IV infusion) with a single dose of either 10 μg of Isotype control (IgG1, n=10) or 10 μg of anti-DEspR 10A3H10 mAb (n=6) at stroke onset (rats were 4-6 months of age with documented neurological deficits). Rats were allowed to proceed to recovery up to eventual death. As shown in the figure, there is a significant increase in post-stroke survival upon anti-DEspR treatment (Mean post-stroke survival time for untreated controls=2.35±1.27 days versus Mean post-stroke survival time for anti-DEspR treated group=25.5±7.3 days; P=0.0007, Gehan-Breslow Test) extending post-stroke survival >ten-fold compared with littermate, genetically identical non-treated controls.

FIGS. 30A-30E show an analysis of blood pressure, heart rate and activity by radiotelemetry in Dahl S female rats injected with 10A3H10 mAb at 16 weeks of age. (30A) Mean systolic blood pressure ±sem (SBP; mmHg). (30B) Mean diastolic blood pressure ±sem (DBP; mmHg). (30C) Mean mean arterial pressure ±sem (MAP; mmHg). (30D) Mean heart rate ±sem (beats/min; BPM). (30E) Mean activity ±sem (Counts/min) in 16 weeks old Dahl S female rats (n=6). At day 7 of continuous monitoring BP (see arrow) subjects were injected (IV) with 10A3H10 mAb at 40 μg/kg of body weight.

### DETAILED DESCRIPTION

Certain aspects described herein are based, in part, on the discovery by the inventors that DEspR contributes to adult tissue vascularity, as well as playing a critical role in angiogenesis during embryonic development, and a critical role in pathological vascularization such as is common in stroke and in the presence of micro-hemorrhages. The inventors also discovered that inhibition of DEspR can reduce damage resulting from stroke or resulting from, or likely to result from, micro-hemorrhages. The inventors also discovered that DEspR is surprisingly expressed in certain tumor cells, cancer stem cells or stem-like cells, or tumor initiating cells, as well as in tumor-surrounding blood vessels' endothelial cells, pericytes, and smooth muscle cells. The inventors further discovered that inhibition of DEspR, using DEspR-specific inhibitors, can inhibit a variety of parameters that characterize tumor metastasis, including cell invasiveness, tumor growth, such as tumor volume or tumor mass, as well as parameters that characterize angiogenesis, including neovessel tube length, neovessel branching, and formation of vessel interconnections.

Based on the foregoing, the inventors have developed diagnostics and therapeutics. Described herein are novel compositions comprising DEspR agonists coupled to toxins, including DEspR agonists that are antibodies to DEspR (and DEspR binding portions thereof) coupled to toxin and VEGFsp (and DEspR binding portions thereof) coupled to toxin. Also described herein are anti-VEGF signal peptide (anti-VEGFsp) antibodies and fragments thereof that bind VEGFsp, including human, humanized, monoclonal and polyclonal antibodies and fragments thereof, and VEGFsp fusion proteins. Such antibodies are DEspR antagonists. Also described herein are pharmaceutical preparations containing DEspR inhibitors, DEspR agonists, DEspR antagonists, DEspR agonists coupled to toxins, and antibodies to VEGFsp in methods of use in a variety of applications, including, but not limited to: 1) anti-angiogenesis therapies, treating stroke, inhibiting adverse neurological events, treating cancer, preventing cancer reoccurrence, and anti-angiogenesis approaches relevant to treatment of those vascular diseases where pathological angiogenesis plays a role in pathogenesis or progression, such as in age-related macular degeneration, carotid artery disease, diabetic retinopathy, rheumatoid arthritis, a neurodegenerative disease, Alzheimer's disease, obesity, endometriosis, psoriasis, atherosclerosis, ocular neovascularization, neovascular glaucoma, osteoporosis, or restenosis. Also provided herein are compositions highly suitable for targeted delivery to sites of angiogenesis.

### DEFINITIONS

The term “antibody” is used in the broadest sense and includes monoclonal antibodies (including full length or intact monoclonal antibodies), polyclonal antibodies, multivalent antibodies, multispecific antibodies (e.g., bispecific antibodies), and antibody fragments (see below) so long as they exhibit the desired biological activity and specificity.

An antibody having a “biological characteristic” of a designated antibody is one which possesses one or more of the biological characteristics of that antibody which distinguish it from other antibodies that bind to the same antigen. One biological characteristic is binding to or otherwise blocking an epitope. In order to screen for antibodies which bind to or otherwise block an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

An antibody that is “isolated” is one that has been identified and separated and/or recovered from a component of its natural environment. Contaminant components of its natural environment are materials that would interfere with diagnostic or therapeutic uses for the antibody, and can include enzymes, hormones, and other proteinaceous or nonproteinaceous solutes. In certain embodiments, the antibody will be purified (1) to greater than 95% by weight of antibody as determined by, for example, the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, silver stain. Isolated antibody includes the antibody in situ within recombinant cells since at least one component of the antibody's natural environment will not be present. Ordinarily, however, isolated antibody will be prepared by at least one purification step.

The term “antibody fragment,” as used herein, refer to a protein fragment that comprises only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) the dAb fragment (Ward et al., Nature 341, 544-546 (1989)) which consists of a V_{H} domain; (vii) isolated CDR regions; (viii) F(ab′)₂ fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870).

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. V_{L} refers to the variable domain of the light chain. According to the methods used in this invention, the amino acid positions assigned to CDRs and FRs can be defined according to Kabat. Amino acid numbering of antibodies or antigen binding fragments is also according to that of Kabat.

An “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent as defined throughout the specification or known in the art, e.g., but are not limited to, antibodies to VEGF-A or to the VEGF-A receptor (e.g., KDR receptor or Flt-1 receptor), VEGF-trap, anti-PDGFR inhibitors such as Gleevec™ (Imatinib Mesylate). Anti-angiogensis agents also include native angiogenesis inhibitors, e.g., angiostatin, endostatin, etc. See, e.g., Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5:1359-1364 (1999); Tonini et al., Oncogene, 22:6549-6556 (2003) (e.g., Table 2 listing known antiangiogenic factors); and Sato. Int. J. Clin. Oncol., 8:200-206 (2003) (e.g., Table 1 lists anti-angiogenic agents used in clinical trials).

The term “anti-cancer therapy” refers to a therapy useful in treating cancer. Examples of anti-cancer therapeutic agents include, but are not limited to, e.g., surgery, chemotherapeutic agents, growth inhibitory agents, cytotoxic agents, radiation therapy, agents used in radiation therapy, anti-angiogenesis agents, apoptotic agents, anti-tubulin agents, and other agents to treat cancer, such as anti-HER-2 antibodies (e.g., Herceptin®), anti-CD20 antibodies, an epidermal growth factor receptor (EGFR) antagonist (e.g., a tyrosine kinase inhibitor), HER1/EGFR inhibitor (e.g., erlotinib (Tarceva®)), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention.

The term “antigen” as used herein refers to a molecule that is bound by a binding site on a polypeptide agent, such as an antibody or antibody fragment thereof. Typically, antigens are bound by antibody ligands and are capable of raising an antibody response in vivo. An antigen can be a polypeptide, protein, nucleic acid or other molecule. In the case of conventional antibodies and fragments thereof, the antibody binding site as defined by the variable loops (L1, L2, L3 and H1, H2, H3) is capable of binding to the antigen. The term “antigenic determinant” refers to an epitope on the antigen recognized by an antigen-binding molecule, and more particularly, by the antigen-binding site of said molecule.

The term “avidity” refers to the measure of the strength of binding between an antigen-binding molecule (such as an antibody or antibody fragment thereof described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody or antibody fragment thereof described herein) will bind to their cognate or specific antigen with a dissociation constant (K_{D} of 10⁻⁵ to 10⁻¹² moles/liter or less, and preferably 10⁻⁷ to 10⁻¹² moles/liter or less and more preferably 10⁻⁸ to 10⁻¹² moles/liter (i.e., with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹° M (0.1 nM) to 10⁻⁵ M (10000 nM). The stronger an interaction is, the lower is its K_{D}. Preferably, a binding site on an antibody or antibody fragment thereof described herein will bind to the desired antigen with an affinity less than 500 nM, preferably less than 200 nM, more preferably less than 10 nM, such as less than 500 μM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the variants thereof known per se in the art; as well as other techniques as mentioned herein.

The terms “cancer” and “cancerous” refer to or describe the physiological condition in mammals that is typically characterized by unregulated cell growth. Included in this definition are benign and malignant cancers, as well as dormant tumors or micrometastases. Accordingly, the terms “cancer” or “tumor” as used herein refers to an uncontrolled growth of cells which interferes with the normal functioning of the bodily organs and systems, including cancer stem cells and tumor vascular niches. A subject that has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs. Hematopoietic cancers, such as leukemia, are able to out-compete the normal hematopoietic compartments in a subject, thereby leading to hematopoietic failure (in the form of anemia, thrombocytopenia and neutropenia) ultimately causing death.

A “chemotherapeutic agent” is a chemical compound useful in the treatment of cancer. Examples of chemotherapeutic agents include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB®); inhibitors of PKC-alpha, Raf, H-Ras, EGFR (e.g., erlotinib (TARCEVA®)) and VEGF-A that reduce cell proliferation and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

As used herein, the term “Complementarity Determining Regions” (CDRs), i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain (Kabat et al.), and/or those residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. For example, the CDRH1 of the heavy chain of antibody 4D5 includes amino acids 26 to 35.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “disorder” is any condition that would benefit from treatment with, for example, an antibody or toxin conjugate described herein. This includes chronic and acute disorders or diseases, including those pathological conditions which predispose the mammal to the disorder in question.

The term “epitope” refers to the portion of an antigen to which an antibody binds. The portion can be formed both from contiguous amino acids, or noncontiguous amino acids juxtaposed by tertiary folding of a molecule, dimerization of a molecule, etc. An epitope typically includes at least 3, and more usually, at least 5, about 9, or about 3-10 amino acids, about 5-10 amino acids, about 8-10 amino acids, or about 9-10 amino acids in a unique spatial conformation. An “epitope” includes the unit of structure conventionally bound by an immunoglobulin V_{H}/V_{L} pair. Epitopes define the binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

By “fragment” is meant a portion of a native polypeptide, excluding the whole polypeptide, such as an antibody fragment. A fragment can contain 10, 20, 30, 40, 50, 60, 70, 80, 90, 100, 120, 140, 160, 180, 190, 200 amino acids or more. A fragment can comprise 10-200 amino acids, 10-100 amino acids, 10-50 amino acids, 10-25 amino acids, 10-20 amino acids, 10-15 amino acids, 5-15 amino acids, etc. In the case of antibody fragments, in embodiments the fragments contain enough of the native amino acids of an intact antibody such that they continue to specifically bind their target.

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

The term “Fab” fragment refres to a variable and constant domain of the light chain and a variable domain and the first constant domain (C_{H}1) of the heavy chain. F(ab′)₂ antibody fragments comprise a pair of Fab fragments which are generally covalently linked near their carboxy termini by hinge cysteines between them. Other chemical couplings of antibody fragments are also known in the art.

A “growth inhibitory agent” as used herein refers to a compound or composition which inhibits growth of a cell in vitro and/or in vivo. Thus, the growth inhibitory agent can be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL®, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13.

The term “Humanized” refers to forms of non-human (e.g., murine) antibodies that are chimeric antibodies that are engineered or designed to comprise minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). As used herein, a “composite human antibody” is a specific type of engineered or humanized antibody.

A “human antibody,” “non-engineered human antibody,” or “fully human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous mouse immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody can be prepared via immortalization of human B lymphocytes producing an antibody directed against a target antigen (such B lymphocytes can be recovered from an individual or can have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

The term “Kabat” refers to the numbering of the residues in an immunoglobulin. The numbering for the heavy chain is that of the EU index as in Kabat et al., Sequences of Proteins of Immunological Interest, 5th Ed. Public Health Service, National Institutes of Health, Bethesda, Md. (1987 and 1991), hereinafter “Kabat”, which is also available on the world wide web, and is expressly incorporated herein in its entirety by reference. The “EU index as in Kabat” refers to the residue numbering of the human IgG1 EU antibody.

The word “label” when used herein refers to a detectable compound or composition which is conjugated directly or indirectly to the polypeptide. The label can be itself be detectable (e.g., radioisotope labels or fluorescent labels) or, in the case of an enzymatic label, can catalyze chemical alteration of a substrate compound or composition which is detectable.

The expression “linear antibodies” refers to the antibodies described in Zapata et al., Protein Eng., 8(10):1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies can be highly specific, and are directed against a single determinant on an antigen. The modifier “monoclonal” is not to be construed as requiring production of the antibody by any particular method. For example, the monoclonal antibodies to be used in accordance with the invention can be made by the hybridoma method first described by Kohler et al., Nature 256:495 (1975), or can be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example. A monoclonal antibody can be of any species, including, but not limited to, mouse, rat, goat, rabbit, and human monoclonal antibodies.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

By “reduce or inhibit” is meant the ability to cause an overall decrease preferably of 20% or greater, 30% or greater, 40% or greater, 45% or greater, more preferably of 50% or greater, of 55% or greater, of 60% or greater, of 65% or greater, of 70% or greater, and most preferably of 75%, 80%, 85%, 90%, 95%, or greater. Reduce or inhibit can refer to, for example, reduction or inhibition one or more symptoms of the disorder being treated, the presence or size of metastases or micrometastases, the size of or number of live cells in the primary tumor, the presence or the size of the dormant tumor, or the size or number of the blood vessels in angiogenic disorders. Reduce or inhibit can also refer to halting the further progression of a symptom, a micro-hemmorhage, tumor growth, etc.

The term “selectively binds” or “specifically binds” refers to the ability of an antibody or antibody fragment thereof described herein to bind to a target, such as a molecule present on the cell-surface, with a K_{D} 10⁻⁵ M (10,000 nM) or less, e.g., 10⁻⁶ M, MC M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay. In embodiments of the present invention, an antibody or fragment thereof specifically binds an antigen when it can be used in vivo to target a tissue expressing the antigen such that off-target effects are clinically acceptable or insignificant.

The term “Single-chain Fv” or “scFv” antibody fragments refers to the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “specificity” refers to the number of different types of antigens or antigenic determinants to which an antibody or antibody fragment thereof can bind. The specificity of an antibody or antibody fragment thereof can be determined based on affinity and/or avidity. The affinity, represented by the equilibrium constant for the dissociation (K_{D}) of an antigen with an antigen-binding protein, is a measure of the binding strength between an antigenic determinant and an antigen-binding site on the antigen-binding protein, such as an antibody or antibody fragment thereof: the lesser the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific antigen of interest. Accordingly, an antibody or antibody fragment thereof as defined herein is said to be “specific for” a first target or antigen compared to a second target or antigen when it binds to the first antigen with an affinity (as described above, and suitably expressed, for example as a K_{D} value) that is at least 10 times, at least 100 times, at least 1000 times, and up to 10000 times or more, better than the affinity with which said antibody or antibody fragment thereof binds to another antigen. Antibody affinities can be determined, for example, by a surface plasmon resonance based assay (such as the BIAcore assay described in PCT Application Publication No. WO2005/012359); enzyme-linked immunoabsorbent assay (ELISA); and competition assays (e.g. RIA's), for example.

By “subject” is meant a mammal, including, but not limited to, a human or non-human mammal, such as a bovine, equine, canine, ovine, or feline. Preferably, the subject is a human. Patients are also subjects herein.

The term “target” refers to a biological molecule (e.g., peptide, polypeptide, protein, lipid, carbohydrate) to which a polypeptide domain which has a binding site can selectively bind. The target can be, for example, an intracellular target (e.g., an intracellular protein target) or a cell surface target (e.g., a membrane protein, a receptor protein). Preferably, a target is a cell surface target, such as a cell surface protein.

### DEspR

DEspR, formerly DEAR, was originally cloned from a Dahl salt-sensitive hypertensive rat brain cDNA library and was shown to be a single transmembrane receptor coupled to a Ca2+-mobilizing transduction pathway binding endothelin-1 (ET-1) and angiotensin-II (Ang II) with equivalent affinities (Ruiz-Opazo N. et al. (1998), Molecular characterization of a dual Endothelin-1/Angiotensin II Receptor. Mol. Med. 4: 96-108). Subsequent molecular studies elucidated that the mouse ortholog does not interact with AngII but binds ET-1 and the vascular endothelial growth factor signal peptide (VEGFsp) with equal affinities instead. DEspR−/− double mutant deficiency in mice resulted in embryonic lethality due to impaired vasculogenesis, abnormal angiogenesis and vascular network formation. DEspR−/− embryos also showed abnormal neurogenesis marked by a hyperconvoluted neuroepithelium and dysregulated neural tube differentiation from the telencephalon to myelencephalon (Herrera V L M, et al., (2005), Embryonic lethality in Dear gene deficient mice: new player in angiogenesis. Physiol. Genomics 23: 257-268.). This phenotype is strikingly opposite to the proapoptotic effects observed in the developing neural tube in VEGF+/− deficient mice, although abnormalities in vasculogenesis and angiogenesis are similar (Herrera V L M, et al., (2005)).

Human “DEspR,” means the 85-amino acid dual endothelin/VEGF signal peptide receptor (DEspR) having the human amino acid sequence of: MTMFKGSNEMKSRWNWGSITCIICFTCVGSQLSMSSSKASNFSGPLQLYQRELEIFIV LTDVPNYRLIKENSHLHTTIVDQGRTV (SEQ ID NO: 1), as described by, e.g., Glorioso et al. 2007, together with naturally occurring allelic, splice variants, and processed forms thereof. DEspR is part of the G protein coupled receptor family, and binds to endothelin-1 and to VEGF signal peptide (“VEGFsp”). VEGFsp has the human sequence: MNFLLSWVHWSLALLLYLHHAKWSQA (SEQ ID NO:2). Typically, DEspR as used herein refers to human DEspR.

The term “DEspR” is also used when referring to truncated forms or fragments of the polypeptide. Reference to any such forms of DEspR can be identified in the application, e.g., by “DEspR (1-9).” Accordingly, in some embodiments of all the aspects described herein, DEspR refers to the truncated forms or fragments of the DEspR polypeptide.

As used herein a DEspR “native sequence” or DEspR “wild-type sequence” polypeptide comprises a polypeptide having the same amino acid sequence as a DEspR polypeptide derived from nature. Thus, a native sequence polypeptide can have the amino acid sequence of naturally-occurring polypeptide from any mammal. Such native sequence polypeptide can be isolated from nature or can be produced by recombinant or synthetic means. The term “native sequence” polypeptide specifically encompasses naturally-occurring truncated or secreted forms of the polypeptide (e.g., an extracellular domain sequence), naturally-occurring variant forms (e.g., alternatively spliced forms) and naturally-occurring allelic variants of the polypeptide.

### DEspR Antagonists or Inhibitors

DEspR antagonists are molecules that reduce or inhibit a biological function of DEspR, such as DEspR activity. DEspR antagonists may neutralize, block, inhibit, abrogate, reduce, or interfere with DEspR activity, by inhibiting, for example, its binding to VEGFsp. Such inhibition may be measured directly, such as by measuring the effect of an antagonist on DEspR signaling or indirectly by measuring the effect on a down-stream function of DEspR activiation, such as inhibiting angiogenesis. The Examples below detail methods of measuring the biological function of DEspR. Examples of DEspR inhibitors include, but are not limited to, molecules which block the binding of VEGFsp, ET-1 and/or other ET-1 or VEGFsp-like ligands to DEspR and inhibit DEspR activity, compounds which reduce the amount of DEspR, such as anti-sense or siRNA, or other compounds or agents that inhibit activation of the receptor. Molecules that block or inhibit the binding to DEspR of VEGFsp, ET-1 or other mimetic ligands include molecules that bind DEspR and molecules that bind a ligand of DEspR, such as molecules that bind VEGFsp. Inhibitors including soluble DEspR receptors, peptides containing the DEspR ET-1 and/or VEGFsp binding domains. In some embodiments, the DEspR inhibitor is an anti-DEspR antagonist antibody or antigen-binding fragment thereof, anti-VEGFsp antibody or antigen-binding fragment thereof, a DEspR soluble receptor molecule, a portion of a DEspR receptor, or small molecule that binds specifically to DEspR or VEGFsp, thereby inhibiting, preventing, or sequestering its binding of DEspR to its ligands.

DEspR antagonists include certain anti-DEspR antibodies. Such antibodies or antibody fragments thereof bind specifically to DEspR and reduce or inhibit the biological activity of DEspR. In some embodiments, the DEspR is human DEspR. In some embodiments, the DEspR target comprises an amino acid sequence of SEQ ID NO: 1 or an allelic or splice variant thereof. In some embodiments, the anti-DEspR antibody or antibody fragment thereof is specific for an epitope of DEspR comprising amino acids 1-9 of SEQ ID NO: 1.

DEspR antagonists also include anti-VEGFsp antibodies. The inventors have shown in the Examples below that antibodies to VEGFsp are effective to reduce or inhibit the biological activity of DEspR. DEspR antagonists that are anti-VEGFsp antibodies or DEspR binding fragments thereof include any antibodies or antibody fragments thereof that bind specifically VEGFsp. Such antibodies reduce or inhibit the biological activity of DEspR.

Anti DEspR antagonist antibodies and anti VEGFsp antibodies are described in greater detail below.

### Stroke

In one aspect of the invention, the inventors have discovered that DEspR antagonists are useful in treating stroke and the neurological deficit associated with micro-hemorrhages. Stroke is a manifestation of vascular injury to the brain commonly caused by atherosclerosis or hypertension, and is the third leading cause of death in the United States. Stroke can be categorized into two broad types, “ischemic stroke” and “hemorrhagic stroke”.

Ischemic stroke encompasses thrombotic, embolic, lacunar and hypoperfusion types of strokes. Thrombi are occlusions of arteries created in situ within the brain while emboli are occlusions caused by material from a distant source, such as the heart and major vessels, often dislodged due to myocardial infarct or atrial fibrillation. Less frequently thrombi may also result from vascular inflammation due to disorders such as meningitis. Thrombi or emboli can result from atherosclerosis or other disorders, for example arteritis, and lead to physical obstruction of arterial blood supply to the brain. Lacunar stroke refers to an infarct within non-cortical regions of the brain. Hypoperfusion embodies diffuse injury caused by non-localized cerebral ischemia, typically caused by myocardial infarction and arrhythmia.

Hemorrhagic stroke is caused by intracerebral or subarachnoid hemorrhage, i.e. bleeding into brain tissue, following blood vessel rupture within the brain. Intracerebral and subarachnoid hemorrhage are subsets of a broader category of hemorrhage referred to as intracranial hemorrhage. Intracerebral hemorrhage is typically due to chronic hypertension, and a resulting rupture of an arteriosclerotic vessel. Stroke associated symptoms of intracerebral hemorrhage are abrupt with the onset of headache and steadily increasing neurological deficits. Nausea, vomiting, delirium seizures and loss of consciousness are additional common stroke-associated symptoms.

In contrast most subarachnoid hemorrhage is caused by head trauma or aneurysm rupture which is accompanied by high pressure blood release which also causes direct cellular trauma. Prior to rupture, aneurysms may be asymptomatic or occasionally associated with tension or migraine headaches. However headache typically becomes acute and severe upon rupture, and may be accompanied by varying degrees of neurological deficit, vomiting, dizziness and altered pulse and respiratory rates.

As shown in the Examples below, the inventors have discovered and demonstrated that inhibition of DEspR leads to marked improvement in neurological parameters post-stroke in an established animal model of stroke that closely mimics aspects of the disorder seen in humans. Significantly, the inventors have demonstrated improvement in post-stroke survival in animals experiencing a stroke and receiving DEspR antagonist therapy, versus control animals experiencing a stroke and not reveiving DEspR antagonist therapy. One of the rat stroke models described herein, the Tg25sp model, is an animal model of ischemic-hemorrhagic stroke that is induced by early life sodium exposure, e.g., on 0.4% NaCl normal rat chow.

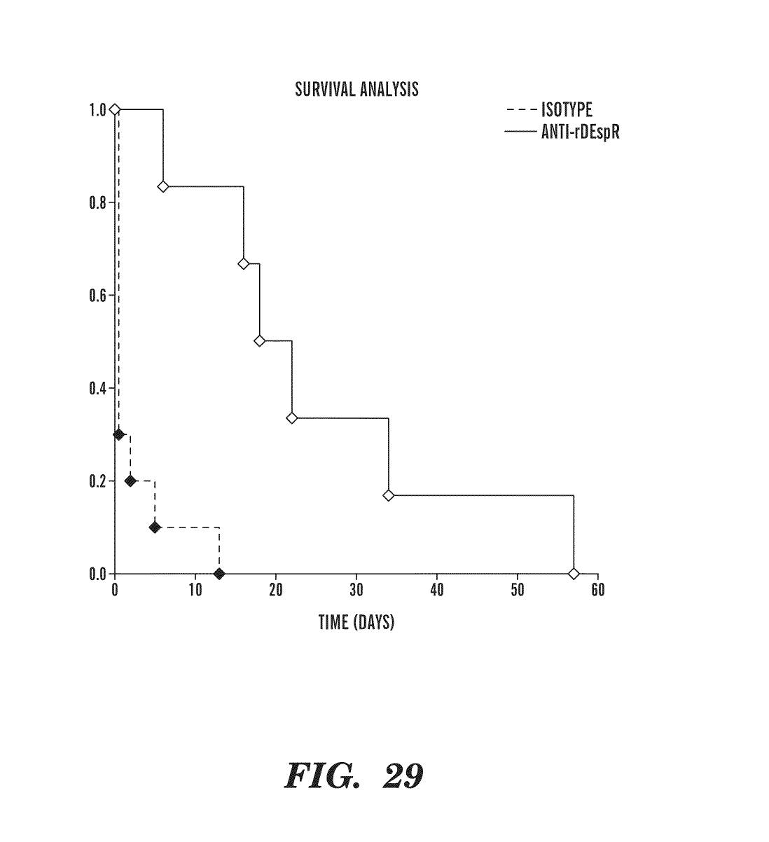

The Tg25sp model is an inbred polygenic-hypertensive, transgenic-hyperlipidemic Dahl salt-sensitive (Dahl-S) rat, with sex-specific differences, and is characterized by the presence of both hypertension and hyperlipidemia as risk factors, and by a disease-course continuum that recapitulates multiple paradigms of human stroke, such as nonocclusive carotid artery disease with chronic low-flow ischemia, microhemorrhages (usually asymptomatic as observed in humans), and hemorrhagic infarctions. Because the stroke-prone model used herein is a genetically identical inbred transgenic rat strain exhibiting a relatively confined temporal disease course, the prestroke stage can be defined and studied, thereby validating the study of prestroke events, as described herein. In this model, one can monitor the onset of spontaneous strokes, which present with unequivocal neurologic deficits followed by death within 24 hours. This experimental design recapitulates the clinical scenario when a patient presents with acute onset of neurologic deficits due to a stroke. As shown in FIG. 29, a significant increase in post-stroke survival was observed upon anti-DEspR treatment, extending post-stroke survival greater than ten-fold compared with littermate, genetically identical non-treated controls.

Tg25sp rat model provides several advantages as an animal model. As a model of spontaneous stroke that recreates two key risk factors, hypertension and hyperlipidemia, it provides an experimental system to investigate new targets for intervention and prevention strategies, as well as an animal model to test new drugs and diagnostic modalities with systematic histological corroboration. These critical tasks are impossible to perform in human studies. As a complex stroke model that spans a spectrum of carotid artery disease, ischemic lesions, microhemorrhages, intraparenchymal hemorrhages, and microvascular alterations, stroke-prone Tg25 rats prove, through reproducible modeling, that the association of these same pathologies in humans is causally interrelated rather than just coincidence, thus giving experimental support for systematic study to determine prognosis for future stroke events. (Decano et al., Circulation. 2009; 119: 1501-1509)

Based on our experimental data in the Examples, and on other information provided herein, methods are provided for treating stroke acutely in a subject, such as human. The stroke may be hemorrhagic or ischemic. In addition, methods are provided for inhibiting adverse neurological events. The methods involve administration to a subject in need of treatment a DEspR antagonist.

In some embodiments, the DEspR antagonistsare administered acutely, for example, within four days of the subject having a stroke. In some embodiments, the DEspR antagonists are administered anytime within two days of the subject having a stroke. In some embodiments, the DEspR antagonists may be administered any time up to 1 day of the subject having a stroke. In some embodiments, the DEspR antagonists are administered between 1 and 24 hours of the subject having a stroke. In some embodiments, the DEspR antagonists are is administered within 12 hours of the subject having a stroke. The DEspR antagonists may also be administered immediately after the subject has had a stroke.

In some embodiments, provided herein are methods of inhibiting an adverse neurological event by administering to a human subject having or suspected of having micro-hemorrhages a DEspR antagonist in an amount effective to inhibit the adverse neurological event. Adverse neurological events include those induced by stroke, cerebral ischemia and especially ischemic events that are caused by insufficient supply of oxygen to the brain. These events can be focalized in a particular region of the brain as occurs in a stroke or a transient ischemic attack. The adverse neurological event may be characterized by further micro-hemorrhages, recurrent cerebral hemorrhage and/or neurological deficit. Treament in some embodiments is acute treatment, as described above. Treatment in some embodiments is based on a clinical diagnosis of the presence of micro-hemorrhages, and is not necessarily acute treatment.

A subject having or suspected of having micro-hemorrhages can be identified by detecting hemorrhages of the cerebral vasculature by imaging techniques, clinical evaluation or the like. Cerebral microhemorrhage results from underlying small vessel pathologies such as hypertensive vasculopathy or CAA. Cerebral microhemorrhages, best visualized by MRI, result from rupture of small blood vessels. Other potential diagnostics include changes in intracranial pressure which may be detected by specific MRI techniques (Glick et al 2006 Alperin) or other standard techniques as described in Method of detecting brain microhemmorhage (U.S. Pat. No. 5,951,476).

An amount effective to treat stroke is that amount necessary to slow, halt or reverse the progression of one or more symptoms arising from the stroke. An amount effective to inhibit an adverse neurological event is that amount necessary to slow, halt or reverse the progression of one or more symptoms of the adverse neurological event, such symptoms including, for example, micro-hemorrhages, recurrent cerebral hemorrhage and/or neurological deficit.

### Anti-VEGFsp Antibodies

The inventors demonstrate experimentally for the first time that anti-VEGFsp antibodies administered in vitro inhibit DEspR activity to a significant extent. In particular, the inventors demonstrate that anti-VEGFsp antibodies appear to work as well as anti-DEspR antibodies in inhibiting angiogenesis in HUVECs and HMECS angiogenesis assays.

Anti-VEGFsp antibodies or antibody fragments thereof that may be useful in the compositions and methods described herein include antibodies or antibody fragments thereof that bind with sufficient affinity and specificity to VEGFsp, i.e., are specific for VEGFsp, and can either reduce or inhibit the biological activity of DEspR. In some such embodiments, the antibodies specifically bind human VEGFsp. In some embodiments, the VEGFsp target has a sequence comprising SEQ ID NO:2 or an allelic variant thereof. In some embodiments, anti-VEGFsp antibodies or antibody fragments thereof include, but are not limited to, monoclonal anti-VEGFsp antibodies, and human, humanized or chimeric antibodies or antibody fragments thereof. In some embodiments, the antibody has an Fc region modified to promote clearance from circulation of the antibody.

According to one aspect of the invention, a preparation of an isolated human or humanized antibody or fragment thereof that binds selectively VEGFsp is provided. Such a preparation may be combined with a pharmaceutically acceptable carrier to form a pharmaceutical preparation constructed and arranged for administration to a human. Such preparations may be, for example, in a physological saline solution and may include buffers, anti-oxidants, chelating agents and the like, as are characteristic of antibody preparations. Such preparations may be in unit dosage forms, containing a unit dosage for administration to a human. Such preparations may be sterile, such as for intraveneous injection or infusion. In some embodiments, the antibody or fragment thereof is a monoclonal antibody. The antibody or fragment thereof may block binding of VEGFsp to DEspR. Theantibody or fragment thereof may sequester VEGFsp and inhibit binding of VEGFsp to DEspR. In some embodiments, the pharmaceutical preparation comprises the antibody. In some embodiments, the pharmaceutical preparation comprises the fragment. In some embodiments, the antibody has an Fc region modified to promote clearance from circulation of the antibody.

In some embodiments of the invention, the anti-VEGFsp antibodies and VEGFsp binding fragments thereof are used in the treatment of stroke, as described above. In some embodiments of the invention, the anti-VEGFsp antibodies and VEGFsp binding fragments thereof are used to inhibit adverse neurological events, as described above. In some embodiments of the invention, the anti-VEGFsp antibodies and VEGFsp binding fragments thereof are used in the treatment of cancer. In some embodiments of the invention, the anti-VEGFsp antibodies and VEGFsp binding fragments thereof are used to inhibit pathological angiogenesis. In general, the anti-VEGFsp antibodies and VEGFsp binding fragments thereof are used whenever it is desirable to use a DEspR inhibitor, as described herein.

### Anti-DEspR Antibodies

In some aspects, provided herein is an anti-DEspR antibody or antibody fragment thereof that is specific for a DEspR target, where the anti-DEspR antibody or antibody fragment thereof specifically binds to the DEspR target. Anti-DEspR antibodies or antibody fragments thereof that may be useful in the compositions and methods described herein include antibodies or antibody fragments thereof that bind with sufficient affinity and specificity to DEspR, i.e., are specific for DEspR, and can either reduce or inhibit the biological activity of DEspR or activate the biological activity of DEspR. In some embodiments, the anti-DEspR antibody is a DEspR antagonist and reduces or inhibits DEspR biological activity. In some embodiments, the anti-DEspR antibody is a DEspR agonist and activates DEspR biological activity. In some embodiments, the DEspR is human DEspR. In some embodiments, the DEspR target comprises an amino acid sequence of SEQ ID NO:1 or an allelic or splice variant thereof.

As used herein, an “antibody” that binds a target refers to an antibody that binds its target with sufficient affinity and specificity to perform the desired function. As used herein, “selectively binds” or “specifically binds” refers to the ability of an antibody or antibody fragment thereof described herein to bind its target, with a K_{D} 10⁻⁵ M (10000 nM) or less, e.g., 10⁻⁶ M, 10⁻⁷ M, 10⁻⁸ M, 10⁻⁹ M, 10⁻¹° M, 10⁻¹¹ M, 10⁻¹² M, or less. In some embodiments, the antibody that binds specifically to DEspR reduces or inhibits the biological function of DEspR, i.e. is a DEspR antagonist. Such an antibody may, for example, inhibits the ability of DEspR to induce angiogenesis, to induce vascular endothelial cell proliferation or to induce vascular permeability. Such an antibody can bind remote from the VEGFsp binding site, but alter configuration of the VEGFsp binding site such that VEGFsp binding is inhibited. Such an antibody can bind remote from the VEGFsp binding site, but alter configuration of DEspR whereby signal transduction via DEspR is inhibited. Such an antibody also might bind the VEGFsp binding site and block of inhibit the binding of VEGFsp to DEspR. DEspR antagonist antibodies, however, exclude DEspR agonists.

In some embodiments, the antibody that binds specifically to DEspR is a DEspR agonist. Such an antibody may bind the same site as VEGFsp and activate DEspR. In other words, it has the same or similar effect on DEspR as does binding of VEGFsp to DEspR, and although the antibody might also block the binding of VEGFsp to DEspR, the overall effect in vivo of such an antibody is to activate rather than inhibit DEspR. Whether a DEspR antibody (or fragment thereof) is a DEspR agonist, therefore, needs to be tested in vivo using assays such as those described herein to determine whether the antibody acts in a physiological setting as an agonist. DEspR agonists are described generally in more detail below. In certain aspects described herein, an anti-DEspR antibody can be used as a therapeutic agent in targeting and interfering with diseases or conditions where DEspR activity is involved. Also, the anti-DEspR antibody can be subjected to other biological activity assays, e.g., in order to evaluate its effectiveness as a therapeutic, or its effectiveness as a diagnostic aid, etc. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). Other biological activity assays that can be used to assess an anti-DEspR antibody are described herein in the Examples section.

### Angiogenesis and Cancer

### Anti-VEGFsp

As discussed above the inventors demonstrate experimentally for the first time that anti-VEGFsp antibodies administered in vitro inhibit DEspR activity to a significant extent. In particular, the inventors demonstrate that anti-VEGFsp antibodies appear to work as well as anti-DEspR antibodies in inhibiting angiogenesis in HUVECs and HMECs angiogenesis assays, and would, just like anti-DEspR antibodies, inhibit tumor growth in an animal model of cancer. The invention, therefore, in some aspects, provides methods of treating cancer. The method in some embodiments comprises administering to a subject having a cancer expressing DEspR an antibody or fragment thereof that binds selectively to VEGFsp in an amount effective to inhibit the cancer. The antibody or fragment thereof may be a monoclonal antibody or a human or humanized monoclonal antibody. In some embodiments, the antibody has an Fc region modified to promote clearance from circulation of the antibody. The invention in other aspects involves methods of inhibiting angiogenesis. Such methods in some embodiments involves administering to a subject having a disease or disorder dependent on or modulated by angiogenesis, an antibody or fragment thereof that binds selectively VEGFsp in an amount effective to inhibit the angiogenesis. In some embodiments, the disease or disorder is cancer, age-related macular degeneration, carotid artery disease, diabetic retinopathy, rheumatoid arthritis, a neurodegenerative disease, Alzheimer's disease, obesity, endometriosis, psoriasis, atherosclerosis, ocular neovascularization, neovascular glaucoma, osteoporosis, or restenosis. The invention in still other aspects involves inhibiting directly the growth of a tumor cell. Methods are provided for inhibiting tumor growth and reducing tumor size or tumor metastasis in a subject having a tumor or metastasis by inhibiting DEspR expression and/or function in a cell. Such methods involve administering to a subject in need thereof a therapeutically effective amount of a pharmaceutical composition comprising any of the anti-VEGFsp antibodies or antibody fragments thereof. In some embodiments of these aspects, the DEspR expression and/or function is inhibited in a tumor cell, a tumor initiating cell, a cancer stem-like cell, a cancer stem cell, a metastatic tumor cell, an endothelial progenitor cell, an inflammatory cell, a tumor stromal cell, a tumor vasculature cell, or any combination thereof. In some such embodiments, the tumor vasculature cell is an endothelial cell, a pericyte, a smooth muscle cell, an adventitial cell, or any combination thereof. In some embodiments of these aspects, the toxin kills a tumor cell, a tumor initiating cell, a cancer stem-like cell, a cancer stem cell, a metastatic tumor cell, an endothelial progenitor cell, an inflammatory cell, a tumor stromal cell, a tumor vasculature cell, or any combination thereof. In some such embodiments, the tumor vasculature cell is an endothelial cell, a pericyte, a smooth muscle cell, an adventitial cell, or any combination thereof.

### DEspR Agonists

DEspR agonists enhance the biological function of DEspR, such as by increasing DEspR activity. DEspR agonists include, but are not limited to, anti-DEspR antibodies and antigen-binding fragments thereof that activiate DEspR, such as those described above. DEspR agonists also include VEGFsp and DEspR-binding fragments thereof, endothelin-land DEspR binding fragments thereof, small molecule mimetics of VEGFsp and endothelin-1, and the like.

Anti-DEspR agonist antibodies or antibody fragments thereof that are useful in the compositions and methods described herein include any antibodies or antibody fragments thereof that bind with sufficient affinity and specificity to DEspR, i.e., are specific for DEspR, and can enhance a biological function of DEspR. In some embodiments, the antibody or fragment thereof binds human DEspR. In some embodiments, the DEspR target comprises an amino acid sequence of SEQ ID NO: 1 or an allelic or splice variant thereof. In some embodiments, the DEspR agonist is a VEGF signal peptide, having the human sequence MNFLLSWVHWSLALLLYLHHAKWSQA (SEQ ID NO:2).

### Agonist Conjugates

The invention also embraces methods for treating diseases or disorders using a DEspR agonist coupled to a toxin. In some embodiments, the disease or disorder is cancer, age-related macular degeneration, carotid artery disease, diabetic retinopathy, rheumatoid arthritis, a neurodegenerative disease, Alzheimer's disease, obesity, endometriosis, psoriasis, atherosclerosis, ocular neovascularization, neovascular glaucoma, osteoporosis, or restenosis. The invention in some aspects involves inhibiting directly the growth of a tumor cell. The invention in some aspects involves inhibiting angiogenesis. DEspR agonists coupled to a toxin can be used to inhibiting tumor growth, reducing tumor size, inhibit tumor metastasis, In some embodiments of these aspects, the DEspR expression and/or function is inhibited in a tumor cell, a tumor initiating cell, a cancer stem-like cell, a cancer stem cell, a metastatic tumor cell, an endothelial progenitor cell, an inflammatory cell, a tumor stromal cell, a tumor vasculature cell, or any combination thereof. In some such embodiments, the tumor vasculature cell is an endothelial cell, a pericyte, a smooth muscle cell, an adventitial cell, or any combination thereof. In some embodiments of these aspects, the toxin kills a tumor cell, a tumor initiating cell, a cancer stem-like cell, a cancer stem cell, a metastatic tumor cell, an endothelial progenitor cell, an inflammatory cell, a tumor stromal cell, a tumor vasculature cell, or any combination thereof. In some such embodiments, the tumor vasculature cell is an endothelial cell, a pericyte, a smooth muscle cell, an adventitial cell, or any combination thereof. In some embodiments, the tumor cells are in a subject who had had one or more of (i) radiation treatment for cancer, (ii) chemotherapy for cancer, or (iii) surgical treatment for cancer.

The DEspR agonist may be an antibody or fragment thereof that binds DEspR, and can activate the biological activity of DEspR as described herein. In some embodiments, the antibody or fragment thereof is a monoclonal antibody. In some embodiments, the antibody or fragment thereof is a human or humanized monoclonal antibody. In some embodiments, the antibody or fragment thereof blocks binding of VEGFsp to DEspR, but the overall effect of such an antibody is to activate the bilogical activity of DEspR. In some embodiments, the DEspR agonist is VEGFsp or a fragment of VEGFsp that binds DEspR. The connection between the DEspR agonist and the toxin can be either covalent or non-covalent, including but not limited to ionic bonds, hydrogen bonding, and hydrophobic/hydrophilic interactions.

In some embodiments, the DEspR agonist is conjugated to a radiotoxin. A radiotoxin is meant to refer to any moiety containing a radioactive isotope. Examples of radioactive isotopes that can be conjugated to antibodies for use diagnostically or therapeutically include but are not limited to ²¹¹At, ⁶⁷Cu, ¹³¹I, ¹²⁵I, ^{9O}Y , ¹⁸⁶Re, ¹⁸⁸Rc, 1⁵³Sm, ²¹²Bi, ³²P, and ²¹²Pb. Methods for preparing radioimmunconjugates are routine in the art.

In some embodiments, the DEspR agonist is conjugated to a chemotoxin. Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. Conjugates of VEGFsp or a fragment thereof and a cytotoxic agent can also be made using any of a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In some embodiments, the DEspR agonist is coupled to a particle that is coupled to, coated with, embedded with or contains the toxin. In some embodiments, the particle is a solid polymer matrix or a liposome.

DEspR agonists can be conjugated or coupled to virtually any agent to target the agent to a DEspR expressing cell. DEspR agonists can be conjugated, for example, to other agents such as any small molecule, an siRNA, a nanoparticle, a targeting agent or an imaging agent (e.g., a microbubble). Such conjugates can be used, for example, in diagnostic, theranostic, or targeting methods. In some embodiments, the agonist is VEGFsp or a DEspR binding fragment thereof. In other embodiments, the agonist is an anti-DEspR antibody agonist or DEspR binding fragment thereof.

Conjugates of the agonists described herein (including VEGFsp, DEspR binding fragments thereof, anti-DEspR antibody agonists and DEspR binding fragments thereof, and a cytotoxic agent can be made using any of a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In other embodiments, the DEspR agonist, can be conjugated to a “receptor” (such as, for example, streptavidin) for utilization in tumor pretargeting wherein the antibody-receptor conjugate is administered to the subject, followed by removal of unbound conjugate from the circulation using a clearing agent and then administration of a “ligand” (e.g. avidin) which is conjugated to a cytotoxic agent (e.g. a radionucleotide). In some embodiments, the agonist, including VEGFsp and DEspR binding fragments thereof, can be conjugated to biotin, and the biotin conjugated antibody or antibody fragment thereof can be further conjugated or linked to a streptavidin-bound or -coated agent, such as a streptavidin-coated microbubble, for use in, for example, molecular imaging of angiogenesis.

The agonists described herein (including VEGFsp, DEspR binding fragments thereof, anti-DEspR antibody agonists and DEspR binding fragments thereof) can also be coupled to liposomes. Liposomes containing the agonist are prepared by methods known in the art, such as described in Epstein et al., Proc. Natl. Acad. Sci. USA, 82:3688 (1985); Hwang et al., Proc. Natl. Acad. Sci. USA, 77:4030 (1980); and U.S. Pat. Nos. 4,485,045 and 4,544,545. Liposomes with enhanced circulation time are disclosed in U.S. Pat. No. 5,013,556.

Particularly useful liposomes can be generated, for example, by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Peptides, including Fab′ fragments of an antibody of the invention can be conjugated to the liposomes as described in Martin et al. J. Biol. Chem. 257: 286-288 (1982) via a disulfide interchange reaction. A chemotherapeutic agent is optionally contained within the liposome. See Gabizon et al. J. National Cancer Inst. 81(19)1484 (1989).

### Cancer Reoccurrence

In some aspects, provided herein are methods for reducing cancer re-occurrence comprising administering to a subject after the subject has had one or more of (i) radiation treatment for cancer, (ii) surgical treatment for cancer and (iii) chemotherapy treatment for cancer, a DEspR inhibitor in an amount effective to reduce cancer re-occurrence. DEspR inhibtors include, but are not limited to, anti-DEspR antagonist antibodies and antigen-binding fragments thereof, anti-VEGFsp antibodies and antigen-binding fragments thereof, DEspR agonists coupled to agents such as toxins and antiangiogenesis agents, small molecules, nanoparticles, polyplex combinations and derivatives thereof that bind specifically to DEspR thereby inhibiting, preventing, or sequestering its binding to its ligands, such as VEGFsp and endothelin-1.

### Circulating Tumor Cell Identification

In some aspects, provided herein are methods for identifying a circulating tumor cell comprising contacting a circulating tumor cell expressing DEspR with an agent that binds DEspR, and detecting the agent bound to the circulating tumor cell. In some embodiments, the agent is an antibody that binds DEspR. In some embodiments, the agent is VEGFsp. In some such embodiments, the VEGFsp is human VEGFsp. In some such embodiments, the VEGFsp target has a sequence comprising SEQ ID NO: 2 or an allelic variant thereof. In some embodiments, the DEspR is human DEspR. In some embodiments, the DEspR target comprises an amino acid sequence of SEQ ID NO: 1 or an allelic or splice variant thereof. In some embodiments, the anti-DEspR antibody or antibody fragment thereof is specific for an epitope of DEspR comprising amino acids 1-9 of SEQ ID NO: 1. Detection and enumeration of circulating tumor cells (CTCs) is important for patient care for a number of reasons. They may be detectable before the primary tumor thus allowing early stage diagnosis. They decrease in response to therapy so the ability to enumerate CTCs allows one to monitor the effectiveness of a given therapeutic regimen. They can be used as a tool to monitor for recurrence in patients with no measurable disease in the adjuvant setting. For example CTC were found to be present in 36% of breast cancer patients 8-22 years after mastectomy apparently from micrometastases (deposits of single tumor cells or very small clusters of neoplastic cells). Meng et al Clin Can Res 1024: 8152-62, 2004.

In addition CTCs may be used to predict progression free survival (PFS) and overall survival (OS) as the presence number of circulating tumor cells in patients with metastatic carcinoma has been shown to be correlated with both PFS and OS See eg Cristofanilli et al J Clin Oncol 23(1): 1420-1430, 2005 Cristofanilli et al N Engi J Med 351(8): 781-791, 2004.

### Antibodies Generally

Examples of antibodies and antibody fragments thereof, as well as methods of making and characterizing the same, are provided below. Most of the specific examples are directed to anti-DEspR antibodies. The same principles, however, apply with equal force to the preparation of other antibodies described herein, such as anti-VEGFsp antibodies.

### Polyclonal Antibodies

Polyclonal antibodies are preferably raised in animals by multiple subcutaneous (sc) or intraperitoneal (ip) injections of the relevant antigen, e.g., DEspR(1-9) and an adjuvant. It can be useful, in some embodiments, to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C=NR, where R and R¹ are different alkyl groups.

Animals can be immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

### Monoclonal Antibodies

Preferably, anti-DEspR antibodies or antibody fragments thereof for use with the compositions and methods described herein are anti-DEspR monoclonal antibodies or fragments thereof. The term “monoclonal antibody” refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. Various methods for making monoclonal antibodies specific for DEspR as described herein are available in the art. For example, the monoclonal antibodies can be made using the hybridoma method first described by Kohler et al., Nature, 256:495 (1975), or by recombinant DNA methods (U.S. Pat. No. 4,816,567). “Monoclonal antibodies” can also be isolated from phage antibody libraries using the techniques described in Clackson et al., Nature 352:624-628 (1991) or Marks et al., J. Mol. Biol. 222:581-597 (1991), for example.

The term anti-DEspR “antibody fragment” refers to a protein fragment that comprises at least an antigen binding site of the intact antibody and thus retaining the ability to bind antigen. Examples of antibody fragments encompassed by the term antibody fragment include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) the dAb fragment (Ward et al., Nature 341, 544-546 (1989)) which consists of a V_{H} domain; (vii) isolated CDR regions; (viii) F(ab′)₂ fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870).

In the hybridoma method of making an anti-DEspR monoclonal antibody, a mouse or other appropriate host animal, such as a hamster or macaque monkey, is immunized as described herein to elicit lymphocytes that produce or are capable of producing antibodies that will specifically bind to the DEspR protein or fragment thereof used for immunization. Alternatively, lymphocytes can be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones can be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells can be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies is described in more detail below.

### Anti-DEspR Hybridomas and Antagonist Monoclonal Antibodies Thereof

In certain aspects described herein, anti-DEspR monoclonal antagonist antibodies include, but are not limited to, the monoclonal anti-DEspR antibody 7C5B2 produced or expressed by the hybridoma 7C5B2 described herein, and referred to as the “7C5B2 antibody,” and derivatives or antigen-binding fragments thereof, including, for example, a “7C5B2 variable heavy chain,” or a “7C5B2” variable light chain.

As described herein, the 7C5B2 hybridoma produces a monoclonal antibody, termed herein as the “7C5B2 anti-DEspR antibody” or “7C5B2 antibody,” that is highly specific for DEspR and can potently inhibit DEspR biological activity. The biological characteristics of the 7C5B2 anti-DEspR antibody render it particularly useful for the compositions and methods described herein, including therapeutic and diagnostic applications. Accordingly, sequence analysis of the 7C5B2 antibody was performed, as described herein, to identify the heavy and light chain variable domain sequences, and complementarity determining region (CDR) sequences, of the 7C5B2 antibody for use in the compositions and methods described herein.

Throughout the present specification and claims, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), which is also available on the world wide web, and is expressly incorporated herein in its entirety by reference. The “EU index as in Kabat” refers to the residue numbering of the human IgG1 EU antibody.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. V_{L} refers to the variable domain of the light chain. According to the methods used herein, the amino acid positions assigned to CDRs and FRs can be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions” (CDRs), i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some embodiments, a complementarity determining region can include amino acids from both a CDR region defined according to Kabat and a hypervariable loop.

The nucleotide sequence encoding the V_{H} or variable domain of the heavy chain of the 7C5B2 antibody, as obtained by sequence analysis of sequences obtained from the 7C5B2 hybridoma, is: CAGGTGCAACTGAAGGAGTCAGGACCTGGCCTGGTGGCGC C C T C A C A G A G C C T G T C C A T T A C C T G C A C T G T C T C T G G G T T C T C A T T A A C C A G C T A T G A T A T A A G C T G G A T T C G C C A G C C A C C A G G A A A G G G T C T G G A G T G G C T T G G A G T A A T A T G G A C T G G T G G A G G C A C A A A T T A T A A T T C A G C T T T C A T G T C C A G A C T G A G C A T C A G C A A G G A C A A C T C C A A G A G C C A A G T T T T C T T A A A A A T G A A C A G T C T G C A A A C T G A T G A C A C A G C C A T A T A T T A C T G T G T A A G A G A T C G G G A T T A C G A C G G G T G G T A C T T C G A T G TCTGGGGCGCAGGGACCACGGTCACCGTCTCCTCA (SEQ ID NO: 3).

The corresponding amino acid of the V_{H} domain of the 7C5B2 antibody is: Q V Q L K E S G P G L V A P S Q S L S I T C T V S G F S L T S Y D I S W I R Q P P G K G L E W L G V I W T G G G T N Y N S A F M S R L S I S K D N S K S Q V F L K M N S L Q TDDTAIYYCVRDRDYDGWYFDVWGAGTTVTVSS (SEQ ID NO: 4).

The 10 amino acid complementarity determining region 1 or CDR1 of the V_{H} domain of the 7C5B2 antibody is: G F S L T S Y DI S (SEQ ID NO: 5). The 16 amino acid CDR2 of the V_{H} domain of the 7C5B2 antibody is: V I W T G G G T N Y N S A F M S (SEQ ID NO: 6). The 11 amino acid CDR2 of the V_{H} domain of the 7C5B2 antibody is: DR DYDGWYFDV (SEQ ID NO: 7).

The nucleotide sequence encoding the V_{L} or variable domain of the light chain of the 7C5B2 antibody, as obtained by sequence analysis of sequences obtained from the 7C5B2 hybridoma, is: G A T G T T T T G A T G A C C C A A A C T C C A C T C T C C C T G C C T G T C A G T C T T G G A G A T C A A G C C T C C A T C T C T T G C A G A T C T A G T C A G A G C A T T G T A C A T A G T A A T G G A A A C A C C T A T T T A G A A T G G T A C C T G C A G A A A C C A G G C C A G T C T C C A A A G C T C C T G A T C T A C A A A G T T T C C A A C C G A T T T T C T G G G G T C C C A G A C A G G T T C A G T G G C A G T G G A T C A G G G A C A G A T T T C A C A C T C A A G A T C A G C A G A G T G G A G G C T G A G G A T C T G G G A G T T T A T T A C T G C T T T C A A G G T T C A C A T G T T C C G T A C A C G T T C G G A G G G G G G A C C A A G C T G G A A A T A A A A (SEQ ID NO: 8).

The corresponding amino acid of the V_{L} domain of the 7C5B2 antibody is: D V L M T Q T P L S L P V S L G D Q A S I S C R S S Q S I V H S N G N T Y L E W Y L Q K P G Q S P K L L I Y K V S N R F S G V P D R F S G S G S G T D F T L K I S R V E A E D L G V Y Y C F Q G S H V P Y T F G G G T K L E I K (SEQ ID NO: 9).

The 16 amino acid complementarity determining region 1 or CDR1 of the V_{L} domain of the 7C5B2 antibody is: RSSQSIVHSNGNTYLE (SEQ ID NO: 10). The 7 amino acid CDR2 of the V_{L} domain of the 7C5B2 antibody is: KVSNRFS (SEQ ID NO: 11). The 9 amino acid CDR2 of the V_{L} domain of the 7C5B2 antibody is: FQGSHVPYT (SEQ ID NO: 12).

As shown in Table 1, sequence analysis of the heavy and light chain variable regions of the 7C5B2 antibody indicates strong homology to human germline sequences:

TABLE 1
Antibody Sequence Analysis

[symbol]

| | H Chain | L Chain |
|---|---|---|
| CDR 1 Length | 30aa | 16aa |
| CDR 2 Length | 16aa | 7aa |
| CDR 3 Length | 11aa | 9aa |
| Closest Human | IGHV4. [symbol] *01 (64%) | IGKV2-30*01 (62%) |
| Germline [symbol] | | |
| Closest Human FW1 [symbol] | IGHV4- [symbol] 1*01 (84%) | IGKV2-30*01 (78%) |
| Closest Human FW2 [symbol] | IGHV4-61*01 (93%) | IGKV2-40*01 (93%) |
| Closest Human FW3 [symbol] | IGHV3-66*01 (60%) | IGKV2-30*01 (87%) |
| Closest Human J [symbol] | IGH [symbol] (91%) | IGK [symbol] (50%) |

[symbol]

CDR definition and sequence numbering according to Kabat [symbol]
Germline ID(x) indicated followed by % homology [symbol]
indicates data missing or illegible when filed

Accordingly, in some embodiments of the aspects provided herein, the heavy and/or light chain variable domain(s) sequence(s) of the 7C5B2 antibody, i.e., SEQ ID NO: 3, SEQ ID NO: 4, SEQ ID NO: 8, and/or SEQ ID NO: 9 can be used to generate, for example, human or humanized antibodies, as described elsewhere herein.

In some aspects, monoclonal antibodies that specifically bind to DEspR are provided having one or more biological characteristics of the 7C5B2 monoclonal antibody. As used herein, an antibody having a “biological characteristic” of a designated antibody, such as the 7C5B2 antibody, is one that possesses one or more of the biological characteristics of that antibody which distinguish it from other antibodies that bind to the same antigen.

Accordingly, in some such embodiments of these aspects, having a biological characteristic of the 7C5B2 monoclonal antibody can include having an ED₅₀ value (i.e., the dose therapeutically effective in 50% of the population) at or around the ED₅₀ value of the 7C5B2 antibody for the given population; having an EC₅₀ value (i.e., the dose that achieves a half-maximal inhibition of a given parameter or phenotype) at or around the EC₅₀ value of the 7C5B2 antibody for a given parameter or phenotye. The effects of any particular dosage can be monitored by a suitable bioassay. For example, in some embodiments of these aspects, the given parameter or phenotype to be inhibited by the antibody that specifically binds to DEspR and has one or more biological characteristics of the 7C5B2 antibody can include, but is not limited to, the mean total tube number in an in vitro tubulogenesis assay, the mean total tube length in an in vitro tubulogenesis assay, the mean number of branching points in an in vitro tubulogenesis assay, the mean number of vessel connections in an in vitro tubulogenesis assay, and/or tumor cell invasiveness.

Accordingly, in those embodiments where the phenotype to be inhibited is mean total tube length, as measured using an in vitro tubulogenesis assay, the EC₅₀ value of the monoclonal antibody having a biological characteristic of the 7C5B2 monoclonal antibody is 10 nM or less, 9 nM or less, 8 nM or less, 7 nM or less, 6 nM or less, 5 nM or less, 4 nM or less, 3 nM or less, 2 nM or less, or 1 nM or less. In some such embodiments, the EC₅₀ value of the monoclonal antibody is in the range of 3.0-5.0 nM, in the range of 3.1-4.9 nM, in the range of 3.2-4.8 nM, in the range of 3.3-4.7 nM, in the range of 3.4-4.6 nM, in the range of 3.5-4.5 nM, in the range of 3.6-4.4 nM, in the range of 3.7-4.3 nM, in the range of 3.8-4.2 nM, or in the range of 3.9-4.1 nM. In some embodiments, the EC₅₀ value for inhibiting mean total tube length of the monoclonal antibody having a biological characteristic of the 7C5B2 monoclonal antibody is in the range of 3.8 nM-4.8 nM.

For example, in those embodiments where the phenotype to be inhibited is number of branch points, as measured using an in vitro tubulogenesis assay, the EC₅₀ value of the monoclonal antibody having a biological characteristic of the 7C5B2 monoclonal antibody is 10 nM or less, 9 nM or less, 8 nM or less, 7 nM or less, 6 nM or less, 5 nM or less, 4 nM or less, 3 nM or less, 2 nM or less, or 1 nM or less. In some such embodiments, the EC₅₀ value of the monoclonal antibody is in the range of 3.0-5.0 nM, in the range of 3.1-4.9 nM, in the range of 3.2-4.8 nM, in the range of 3.3-4.7 nM, in the range of 3.4-4.6 nM, in the range of 3.5-4.5 nM, in the range of 3.6-4.4 nM, in the range of 3.7-4.3 nM, in the range of 3.8-4.2 nM, or in the range of 3.9-4.1 nM. In some embodiments, the EC₅₀ value for inhibiting total number of branch points of the monoclonal antibody having a biological characteristic of the 7C5B2 monoclonal antibody is in the range of 3.4 nM-4.5 nM, in the range of 3.5 nM-4.4 nM, in the range of 3.6 nM-4.3 nM, in the range of 3.7 nM-4.2 nM, in the range of 3.8 nM-4.1 nM, in the range of 3.9 nM-4.0 nM.

For example, in those embodiments where the phenotype to be inhibited is tumor cell invasiveness, as measured in vitro, the EC₅₀ value of the monoclonal antibody having a biological characteristic of the 7C5B2 monoclonal antibody is 10 nM or less, 9 nM or less, 8 nM or less, 7 nM or less, 6 nM or less, 5 nM or less, 4 nM or less, 3 nM or less, 2 nM or less, or 1 nM or less. In some such embodiments, the EC₅₀ value of the monoclonal antibody is in the range of 3.0-5.0 nM, in the range of 3.1-4.9 nM, in the range of 3.2-4.8 nM, in the range of 3.3-4.7 nM, in the range of 3.4-4.6 nM, in the range of 3.5-4.5 nM, in the range of 3.6-4.4 nM, in the range of 3.7-4.3 nM, in the range of 3.8-4.2 nM, or in the range of 3.9-4.1 nM. In some embodiments, the EC₅₀ value for inhibiting tumor cell invasivenss of the monoclonal antibody having a biological characteristic of the 7C5B2 monoclonal antibody is in the range of 3.2 nM-3.9 nM, in the range of 3.3 nM-3.8 nM, 3.4 nM-3.7 nM, or in the range of 3.5 nM-3.6 nM.

In some embodiments of the aspects described herein, anti-DEspR antibodies for use in the compositions and methods described herein include monoclonal antibodies that bind to the same epitope or epitopes of DEspR as the monoclonal anti-DEspR 7C5B2 antibody.

In other aspects described herein, anti-DEspR antibodies for use in the compositions and methods described herein include: the monoclonal anti-DEspR antibody 7C5C5 produced or expressed by the hybridoma 7C5C5 described herein, referred to as the “7C5C5 antibody,” and derivatives or fragments thereof; monoclonal antibodies that bind to the same epitope or epitopes of DEspR as the monoclonal anti-DEspR 7C5C5 antibody; the monoclonal anti-DEspR antibody 5G12E8 produced or expressed by the hybridoma 5G12E8 described herein, referred to as the “5G12E8 antibody,” and derivatives or fragments thereof; monoclonal antibodies that bind to the same epitope or epitopes of DEspR as the monoclonal anti-DEspR 5G12E8 antibody; and monoclonal antibodies produced by hybridomas 2E4A8, 2E4B11, 2E4H10, 8E7D11, 8E2F6, E2G4 and 8E7F8.

In addition to generation and production via hybridomas, antibodies or antibody fragments that specifically bind DEspR can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA sequences encoding the antibodies or antibody fragment that specifically bind DEspR also can be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide, as also described elsewhere herein.

Such non-immunoglobulin polypeptides can be substituted for the constant domains of an antibody, or they can be substituted for the variable domains of one antigen-combining site of an antibody to create a chimeric bivalent antibody comprising one antigen-combining site having specificity for an antigen and another antigen-combining site having specificity for a different antigen.

### Humanized and Human Antibodies

Provided herein, in some aspects, are humanized antibodies for use in the compositions and methods described herein. Humanized forms of non-human (e.g., murine) antibodies refer to chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, a humanized antibody can comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also can comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such humanized antibodies are chimeric antibodies (U.S. Pat. No. 4,816,567) where substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. In some embodiments, humanized antibodies comprising one or more variable domains comprising the amino acid sequence of the variable heavy (SEQ ID NO: 4) and/or variable light (SEQ ID NO: 9) chain domains of the murine anti-DEspR antibody 7C5B2, are provided.

Accordingly, as one example in connection with an antagonist antibody for use in some embodiments described herein, one or more heavy and/or one or more light chain CDR regions of a human or humanized anti-DEspR antibody or antibody fragment thereof comprises a sequence of the 7C5B2 antibody described herein. In some such embodiments, the one or more variable heavy chain CDR regions comprises a sequence selected from the group consisting of SEQ ID NO: 5, SEQ ID NO: 6, or SEQ ID NO: 7. In some such embodiments, the one or more variable light chain CDR regions comprises a sequence selected from the group consisting of SEQ ID NO: 10, SEQ ID NO: 11, or SEQ ID NO: 12. In some such embodiments, the one or more variable heavy chain CDR regions comprises a sequence selected from the group consisting of SEQ ID NO: 5, SEQ ID NO: 6, or SEQ ID NO: 7, and the one or more variable light chain CDR regions comprises a sequence selected from the group consisting of SEQ ID NO: 10, SEQ ID NO: 11, or SEQ ID NO: 12.

In some embodiments of the aspects described herein, a human or humanized anti-DEspR monoclonal antibody comprises mutated human IgG1 framework regions and one or more heavy and/or one or more light chain CDR regions from the murine anti-human DEspR monoclonal antibody 7C5B2, described herein, that blocks binding of human DEspR to its ligands. In some such embodiments, the one or more variable heavy chain CDR regions comprises a sequence selected from the group consisting of SEQ ID NO: 5, SEQ ID NO: 6, or SEQ ID NO: 7. In some such embodiments, the one or more variable light chain CDR regions comprises a sequence selected from the group consisting of SEQ ID NO: 10, SEQ ID NO: 11, or SEQ ID NO: 12. In some such embodiments, the one or more variable heavy chain CDR regions comprises a sequence selected from the group consisting of SEQ ID NO: 5, SEQ ID NO: 6, or SEQ ID NO: 7, and the one or more variable light chain CDR regions comprises a sequence selected from the group consisting of SEQ ID NO: 10, SEQ ID NO: 11, or SEQ ID NO: 12.

In some embodiments, a human or humanized anti-DEspR monoclonal antibody comprises mutated human IgG4 framework regions and one or more heavy and/or one or more light chain CDR regions from the murine anti-human DEspR monoclonal antibody 7C5B2, described herein, that blocks binding of human DEspR to its ligands. In some such embodiments, the one or more variable heavy chain CDR regions comprises a sequence selected from the group consisting of SEQ ID NO: 5, SEQ ID NO: 6, or SEQ ID NO: 7. In some such embodiments, the one or more variable light chain CDR regions comprises a sequence selected from the group consisting of SEQ ID NO: 10, SEQ ID NO: 11, or SEQ ID NO: 12. In some such embodiments, the one or more variable heavy chain CDR regions comprises a sequence selected from the group consisting of SEQ ID NO: 5, SEQ ID NO: 6, or SEQ ID NO: 7, and the one or more variable light chain CDR regions comprises a sequence selected from the group consisting of SEQ ID NO: 10, SEQ ID NO: 11, or SEQ ID NO: 12.

The choice of human variable domains, both light and heavy, to be used in making the human or humanized antibodies is very important to reduce antigenicity. According to the so-called “best-fit” method, the amino acid sequences of the variable heavy and light chain domains of a rodent antibody, such as that of the 7C5B2 antibody (SEQ ID NO: 4 and SEQ ID NO: 9, repectively), are screened against the entire library of known human variable-domain sequences. The human sequence which is closest to that of the rodent is then accepted as the human framework (FR) for the human or humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework can be used for several different humanized antibodies (Carter et al., Proc. Natl. Acad. Sci. USA, 89:4285 (1992); Presta et al., J. Immunol., 151:2623 (1993)).

It is further important that antibodies retain high affinity for the antigen and other favorable biological properties, for example, the anti-angiogenic properties of the anti-DEspR antibody 7C5B2 described herein. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Exemplary humanized antibodies and variants thereof directed against the VEGF antigen are described in, for example, U.S. Pat. No. 6,884,879 issued Feb. 26, 2005.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992).

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

Human antibodies can also be generated by in vitro activated B cells (see U.S. Pat. Nos. 5,567,610 and 5,229,275).

### Design & Generation of Composite Human Antibodies

In some embodiments of the aspects described herein, composite human antibody technology that generates de-immunized 100% engineered human antibodies at the outset can be used to prepare humanized composite anti-DEspR antibodies for use in the compositions and methods described herein, using, for example, a technology as described by Antitope.

Briefly, as used herein, “composite human antibodies” comprise multiple sequence segments (“composites”) derived from V-regions of unrelated human antibodies that are selected to maintain monoclonal antibody sequences critical for antigen binding of the starting murine precursor anti-human DEspR monoclonal antibody, such as 7C5B2 antibody, and which have all been filtered for the presence of potential T-cell epitopes using “in silico tools” (Holgate & Baker, 2009). The close fit of human sequence segments with all sections of the starting antibody V regions and the elimination of CD4+ T cell epitopes from the outset allow this technology to circumvent immunogenicity in the development of ‘100% engineered human’ therapeutic antibodies while maintaining optimal affinity and specificity through the prior analysis of sequences necessary for antigen-specificity (Holgate & Baker 2009).

As described herein, structural models of mouse anti-hDEspR antibody V regions were produced using Swiss PDB and analysed in order to identify important “constraining” amino acids in the V regions that were likely to be essential for the binding properties of the antibody. Residues contained within the CDRs (using Kabat definition) together with a number of framework residues were considered to be important. Both the V_{H} and V_{L} (V_{K}) sequences of anti-hDEspR, as described herein as SEQ ID NO: 4 and SEQ ID NO: 9, comprise typical framework residues and the CDR1, CDR2, and CDR3 motifs are comparable to many murine antibodies, as described elsewhere herein.

From the above analysis, it was determined that composite human sequences of anti-hDEspR can be created with a wide latitude of alternatives outside of CDRs but with only a narrow menu of possible alternative residues within the CDR sequences. Analysis indicated that corresponding sequence segments from several human antibodies could be combined to create CDRs similar or identical to those in the murine sequences. For regions outside of and flanking the CDRs, a wide selection of human sequence segments were identified as possible components of novel anti-DEspR composite human antibody V regions for use with the compositions and methods described herein (see, for example, Table 1).

Based upon these analyses, a large preliminary set of sequence segments that could be used to create, as an example, a novel anti-DEspR composite human antibody variants were selected and analysed using iTope™ technology for in silico analysis of peptide binding to human MHC class II alleles (Perry et al 2008), and using the TCED™ (T Cell Epitope Database) of known antibody sequence-related T cell epitopes (Bryson et al 2010). Sequence segments that were identified as significant non-human germline binders to human MHC class II or that scored significant hits against the TCED™ were discarded. This resulted in a reduced set of segments, and combinations of these were again analysed, as above, to ensure that the junctions between segments did not contain potential T cell epitopes. Selected segments were then combined to produce heavy and light chain V region sequences for synthesis.

Accordingly, provided herein are variable heavy and light chain sequences for use in anti-DEspR composite human antibody or engineered human antibody production. In some embodiments, an anti-DEspR composite human antibody can comprise a variable heavy (V_{H}) chain amino acid sequence selected from the group consisting of: Q V Q L Q E S G P G L V K P S Q T L S L T C T V S G F S L T S Y D I S W I R Q P P G K G L E W L G V I W T G G G T N Y N S A F M S R L T I S K D N S K S T V Y L Q M N S L R A E D T A I Y Y C V R D R D Y D G W Y F D V W G Q G T T V T V S S(SEQ ID NO: 13);

### Q V Q L Q E S G P G L V K P S Q T L S L T C T V S G F S L T S Y D I S W I R Q P P G K G L E W L G V I W T G G G T N Y N S A F M S R L T I S K D N S K N T V Y L Q M N S L R A E D T A I Y Y C V R D R D Y D G W Y F D V W G Q G T T V T V S S(SEQ ID NO: 14);

### V Q L Q E S G P G L V K P S Q T L S L T C T V S G F S L T S Y D I S W I R Q P P G K G L E W L G V I W T G G G T N Y N S A F M S R F T I S K D N S K N T V Y L Q M N S L R A E D T A I Y Y C V R D R D Y D G W Y F D V W G Q G T T V T V S S (SEQ ID NO: 15);

Q V Q L Q E S G P G L V K P S Q T L S L T C T V S G F S L T S Y D I S W I R Q P P G K G L E W L G V I W T G G G T N Y N S A F M S R L T I S K D N S K N T V Y L Q M N S L R A E D T A V Y Y C V R D R D Y D G W Y F D V W G Q G T T V T V S S (SEQ ID NO: 16); and

### Q V Q L Q E S G P G L V K P S Q T L S L T C T V S G F S L T S Y D I S W I R Q P P G K G L E W L G V I W T G G G T N Y N S A F M S R F T I S K D N S K N T V Y L Q M N S L R A E D T A V Y Y C V R D R D (SEQ ID NO: 17).

In some embodiments, an anti-DEspR composite human antibody can comprise a variable light (V_{L}) chain amino acid sequence selected from the group consisting of: D V L M T Q S P L S L P V T L G Q P A S I S C R S S Q S I V H S N G N T Y L E W Y L Q K P G Q S P Q L L I Y K V S N R F S G V P D R F S G S G S G T D F T L K I S R V E A E D V G V Y Y C F Q G S H V P Y T F G Q G T K L E I K (SEQ ID NO: 18) and

### D V V M T Q S P L S L P V T L G Q P A S I S C R S S Q S I V H S N G N T Y L E W Y L Q K P G Q S P Q L L I Y K V S N R F S G V P D R F S G S G S G T D F T L K I S R V E A E D V G V Y Y C F Q G S H V P Y T F G Q G T K L E I K (SEQ ID NO: 19).

In some embodiments, an anti-DEspR composite human antibody can comprise a heavy chain CDR1 region comprising an amino acid sequence of SEQ ID NO: 5. In some embodiments, an anti-DEspR composite human antibody can comprise a heavy chain CDR2 region comprising an amino acid sequence of SEQ ID NO: 6. In some embodiments, an anti-DEspR composite human antibody can comprise a heavy chain CDR3 region comprising an amino acid sequence of SEQ ID NO: 7.

In some embodiments, an anti-DEspR composite human antibody can comprise a light chain CDR1 region comprising a sequence of SEQ ID NO: 10. In some embodiments, an anti-DEspR composite human antibody can comprise a light chain CDR2 region comprising an amino acid sequence of SEQ ID NO: 11. In some embodiments, an anti-DEspR composite human antibody can comprise a light chain CDR3 region comprising an amino acid sequence of SEQ ID NO: 12.

### Antibody Fragments

In some embodiments of the aspects described herein, an antibody specific for DEspR, such as, for example the anti-DEspR 7C5B2 antibody; an anti-DEspR antibody comprising one or more heavy chain CDR regions comprising a sequence selected from the group consisting of SEQ ID NO: 5, SEQ ID NO: 6, or SEQ ID NO: 7; an anti-DEspR antibody comprising one or more light chain CDR regions comprises a sequence selected from the group consisting of SEQ ID NO: 10, SEQ ID NO: 11, or SEQ ID NO: 12; an anti-DEspR composite human antibody comprising a variable heavy (V_{H}) chain amino acid sequence selected from the group consisting of SEQ ID NO: 4 and SEQ ID NO: 13-SEQ ID NO: 17; or an anti-DEspR composite human antibody comprising a variable light (V_{L}) chain amino acid sequence selected from the group consisting of SEQ ID NO: 9, SEQ ID NO: 18, and SEQ ID NO: 19 can be treated or processed into an antibody fragment thereof.

Various techniques have been developed and are available for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody fragment of choice is a single chain Fv fragment (scFv). See WO 93/16185.

In some embodiments of the aspects described herein, a human DEspR-specific antibody fragment is a Fab fragment comprising V_{L}, C_{L}, V_{H} and C_{H}1 domains. Fab fragments comprise a variable and constant domain of the light chain and a variable domain and the first constant domain (C_{H}1) of the heavy chain. In some such embodiments, the V_{H} domain is selected from the group consisting of SEQ ID NO: 4 and SEQ ID NO: 13-SEQ ID NO: 17. In some such embodiments, the V_{H} domain comprises one or more heavy chain CDR regions comprising a sequence selected from the group consisting of SEQ ID NO: 5, SEQ ID NO: 6, and SEQ ID NO: 7. In some such embodiments, the V_{L} domain is selected from the group consisting of SEQ ID NO: 9, SEQ ID NO: 18, and SEQ ID NO: 19. In some such embodiments, the V_{L} domain comprises one or more light chain CDR regions comprising a sequence selected from the group consisting of SEQ ID NO: 10, SEQ ID NO: 11, or SEQ ID NO: 12.

In some embodiments of the aspects described herein, a human DEspR-specific antibody fragment is a Fab′ fragment, which is a Fab fragment having one or more cysteine residues at the C-terminus of the C_{H}1 domain.

In some embodiments of the aspects described herein, a human DEspR-specific antibody fragment is an Fd-fragment comprising V_{H} and C_{H}1 domains. In some such embodiments, the V_{H} domain is selected from the group consisting of SEQ ID NO: 4 and SEQ ID NO: 13-SEQ ID NO: 17. In some such embodiments, the V_{H} domain comprises one or more heavy chain CDR regions comprising a sequence selected from the group consisting of SEQ ID NO: 5, SEQ ID NO: 6, and SEQ ID NO: 7.

In some embodiments of the aspects described herein, a human DEspR-specific antibody fragment is a Fd′ fragment comprising V_{H} and C_{H}1 domains and one or more cysteine residues at the C-terminus of the C_{H}1 domain. In some such embodiments, the V_{H} domain is selected from the group consisting of SEQ ID NO: 4 and SEQ ID NO: 13-SEQ ID NO: 17. In some such embodiments, the V_{H} domain comprises one or more heavy chain CDR regions comprising a sequence selected from the group consisting of SEQ ID NO: 5, SEQ ID NO: 6, and SEQ ID NO: 7.

Single-chain Fv or scFv antibody fragments comprise the V_{H} and V_{L} domains of antibody, such that these domains are present in a single polypeptide chain. Generally, a Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994). Accordingly, in some embodiments of the aspects described herein, a human DEspR-specific antibody fragment is a Fv fragment comprising the V_{L} and V_{H} domains of a single arm of an antibody. In some such embodiments, the V_{H} domain is selected from the group consisting of SEQ ID NO: 4 and SEQ ID NO: 13-SEQ ID NO: 17. In some such embodiments, the V_{H} domain comprises one or more heavy chain CDR regions comprising a sequence selected from the group consisting of SEQ ID NO: 5, SEQ ID NO: 6, and SEQ ID NO: 7. In some such embodiments, the V_{L} domain is selected from the group consisting of SEQ ID NO: 9, SEQ ID NO: 18, and SEQ ID NO: 19. In some such embodiments, the V_{L} domain comprises one or more light chain CDR regions comprising a sequence selected from the group consisting of SEQ ID NO: 10, SEQ ID NO: 11, or SEQ ID NO: 12.

The term diabodies refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

Accordingly, in some embodiments of the aspects described herein, a human DEspR-specific antibody fragment is a diabody comprising two antigen binding sites, comprising a heavy chain variable domain (V_{H}) connected to a light chain variable domain (V_{L}) in the same polypeptide chain. In some such embodiments, the V_{H} domain is selected from the group consisting of SEQ ID NO: 4 and SEQ ID NO: 13-SEQ ID NO: 17. In some such embodiments, the V_{H} domain comprises one or more heavy chain CDR regions comprising a sequence selected from the group consisting of SEQ ID NO: 5, SEQ ID NO: 6, and SEQ ID NO: 7. In some such embodiments, the V_{L} domain is selected from the group consisting of SEQ ID NO: 9, SEQ ID NO: 18, and SEQ ID NO: 19. In some such embodiments, the V_{L} domain comprises one or more light chain CDR regions comprising a sequence selected from the group consisting of SEQ ID NO: 10, SEQ ID NO: 11, or SEQ ID NO: 12.

In some embodiments of the aspects described herein, a human DEspR-specific antibody fragment is a dAb fragment comprising a V_{H} domain. In some embodiments, the V_{H} domain is selected from the group consisting of SEQ ID NO: 4 and SEQ ID NO: 13-SEQ ID NO: 17. In some embodiments, the V_{H} domain comprises one or more heavy chain CDR regions comprising a sequence selected from the group consisting of SEQ ID NO: 5, SEQ ID NO: 6, and SEQ ID NO: 7.

In some embodiments of the aspects described herein, a human DEspR-specific antibody fragment comprises isolated CDR regions. In some such embodiments, the isolated CDR region comprises one or more heavy chain CDR regions comprising a sequence selected from the group consisting of SEQ ID NO: 5, SEQ ID NO: 6, and SEQ ID NO: 7. In some such embodiments, the isolated CDR region comprises one or more light chain CDR regions comprising a sequence selected from the group consisting of SEQ ID NO: 10, SEQ ID NO: 11, or SEQ ID NO: 12.

In some embodiments of the aspects described herein, the human DEspR-specific antibody fragment is a F(ab′)₂ fragment, which comprises a bivalent fragment comprising two Fab′ fragments linked by a disulphide bridge at the hinge region.

“Linear antibodies” refers to the antibodies as described in Zapata et al., Protein Eng., 8(10):1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

In some embodiments of the aspects described herein, a human DEspR-specific antibody fragment is a linear antibody comprising a pair of tandem Fd segments (V_{H}-C_{H}1-V_{H} C_{H}1) which, together with complementary light chain polypeptides, form a pair of antigen binding regions. In some such embodiments, the V_{H} domain is selected from the group consisting of SEQ ID NO: 4 and SEQ ID NO: 13-SEQ ID NO: 17. In some such embodiments, the V_{H} domain comprises one or more heavy chain CDR regions comprising a sequence selected from the group consisting of SEQ ID NO: 5, SEQ ID NO: 6, and SEQ ID NO: 7. In some such embodiments, the V_{L} domain is selected from the group consisting of SEQ ID NO: 9, SEQ ID NO: 18, and SEQ ID NO: 19. In some such embodiments, the V_{L} domain comprises one or more light chain CDR regions comprising a sequence selected from the group consisting of SEQ ID NO: 10, SEQ ID NO: 11, or SEQ ID NO: 12.

In other embodiments of these aspects, a human DEspR-specific antibody fragment has specificity for the same epitope as the monoclonal anti-DEspR antibody 7C5B2, described herein, and produced by hybridoma 7C5B2.

Some further examples of DEspR-inhibiting antibodies are described in PCT/US2005/041594, the contents of which are incorporated herein by reference in their entirety. The foregoing technology can be used equally in the production of human or humanized antibodies to VEGFsp.

### Other Amino Acid Sequence Modifications

In some embodiments of the aspects described herein, amino acid sequence modification(s) of the antibodies or antibody fragments thereof described herein are contemplated. For example, it can be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics, e.g., binding specificity, inhibition of biological activity. The amino acid changes also can alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated for use in the antibodies or antibody fragments thereof specific for DEspR or VEGFsp described herein.

Substantial modifications in the biological properties of the antibodies or antibody fragments thereof specific for DEspR or VEGFsp are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (O); (3) acidic: Asp (D), Glu (E); (4) basic: Lys (K), Arg (R), His (H).

Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

Any cysteine residue not involved in maintaining the proper conformation of the antibodies or antibody fragments thereof specific for DEspR or VEGFsp also can be substituted, generally with serine, to improve the oxidative stability of the molecule and prevent aberrant crosslinking Conversely, cysteine bond(s) can be added to the antibody to improve its stability (particularly where the antibody is an antibody fragment such as an Fv fragment).

A particularly preferred type of substitutional variant involves substituting one or more hypervariable region residues of a parent antibody (e.g., the monoclonal anti-DEspR antibody 7C5B2, a monoclonal antibody to VEGFsp or a humanized or human antibody or antibody fragment thereof specific for DEspR or VEGFsp, as provided herein). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding.

Alternatively, or additionally, it can be beneficial to analyze a crystal structure of the antigen-antibody complex to identify contact points between the antibody or antibody fragments thereof specific for anti-DEspR and human DEspR or anti-VEGFsp and VEGFsp. Such contact residues and neighboring residues are candidates for substitution according to the techniques elaborated herein. Once such variants are generated, the panel of variants is subjected to screening as described herein and antibodies or antibody fragments thereof with superior properties in one or more relevant assays can be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine can also be used.

Addition of glycosylation sites to the antibodies or antibody fragments thereof specific for DEspR or VEGFsp is accomplished by altering the amino acid sequence such that it contains one or more of the above-described tripeptide sequences (for N-linked glycosylation sites). The alteration can also be made by the addition of, or substitution by, one or more serine or threonine residues to the sequence of the original antibody (for O-linked glycosylation sites).

Where the antibody comprises an Fc region, the carbohydrate attached thereto can be altered. For example, antibodies with a mature carbohydrate structure that lacks fucose attached to an Fc region of the antibody are described in US Pat Appl No US 2003/0157108 A1, Presta, L. See also US 2004/0093621 A1 (Kyowa Hakko Kogyo Co., Ltd). Antibodies with a bisecting N-acetylglucosamine (GlcNAc) in the carbohydrate attached to an Fc region of the antibody are referenced in WO03/011878, Jean-Mairet et al. and U.S. Pat. No. 6,602,684, Umana et al. Antibodies with at least one galactose residue in the oligosaccharide attached to an Fc region of the antibody are reported in WO97/30087, Patel et al. See, also, WO98/58964 (Raju, S.) and WO99/22764 (Raju, S.) concerning antibodies with altered carbohydrate attached to the Fc region thereof.

In some embodiments, it can be desirable to modify the antibodies or antibody fragments thereof specific for DEspR described herein with respect to effector function, e.g., so as to enhance or diminish antigen-dependent cell-mediated cyotoxicity (ADCC) and/or complement dependent cytotoxicity (CDC) of the antibody. This can be achieved by introducing one or more amino acid substitutions in an Fc region of the antibody or antibody fragment thereof. Alternatively or additionally, cysteine residue(s) can be introduced in the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity can also be prepared using heterobifunctional cross-linkers as described in Wolff et al. Cancer Research 53:2560-2565 (1993). Alternatively, an antibody can be engineered which has dual Fc regions and can thereby have enhanced complement lysis and ADCC capabilities. See Stevenson et al. Anti-Cancer Drug Design 3:219-230 (1989).

For example, WO00/42072 (Presta, L.) describes antibodies with improved ADCC function in the presence of human effector cells, where the antibodies comprise amino acid substitutions in the Fc region thereof. Preferably, the antibody with improved ADCC comprises substitutions at positions 298, 333, and/or 334 of the Fc region (Eu numbering of residues). Preferably the altered Fc region is a human IgG1 Fc region comprising or consisting of substitutions at one, two or three of these positions. Such substitutions are optionally combined with substitution(s) which increase C1q binding and/or CDC.

Antibodies with altered C1q binding and/or complement dependent cytotoxicity (CDC) are described in WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.). The antibodies comprise an amino acid substitution at one or more of amino acid positions 270, 322, 326, 327, 329, 313, 333 and/or 334 of the Fc region thereof (Eu numbering of residues).

To increase the serum half life of the antibody specific for DEspR or VEGFsp described herein, one can incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG.sub.1, IgG.sub.2, IgG.sub.3, or IgG.sub.4) that is responsible for increasing the in vivo serum half-life of the IgG molecule.

Antibodies with improved binding to the neonatal Fc receptor (FcRn), and increased half-lives, are described in WO00/42072 (Presta, L.) and US2005/0014934A1 (Hinton et al.). These antibodies comprise an Fc region with one or more substitutions therein which improve binding of the Fc region to FcRn. For example, the Fc region can have substitutions at one or more of positions 238, 250, 256, 265, 272, 286, 303, 305, 307, 311, 312, 314, 317, 340, 356, 360, 362, 376, 378, 380, 382, 413, 424, 428 or 434 (Eu numbering of residues). The preferred Fc region-comprising antibody variant with improved FcRn binding comprises amino acid substitutions at one, two or three of positions 307, 380 and 434 of the Fc region thereof (Eu numbering of residues). In one embodiment, the antibody has 307/434 mutations.

To increase the half-life of the antibodies or polypeptide containing the amino acid sequences described herein, one can attach a salvage receptor binding epitope to the antibody (especially an antibody fragment), as described, e.g., in U.S. Pat. No. 5,739,277. For example, a nucleic acid molecule encoding the salvage receptor binding epitope can be linked in frame to a nucleic acid encoding a polypeptide sequence described herein so that the fusion protein expressed by the engineered nucleic acid molecule comprises the salvage receptor binding epitope and a polypeptide sequence described herein. As used herein, the term “salvage receptor binding epitope” refers to an epitope of the Fc region of an IgG molecule (e.g., IgG1, IgG₂, IgG₃, or IgG₄) that is responsible for increasing the in vivo serum half-life of the IgG molecule (e.g., Ghetie et al., Ann Rev. Immunol. 18:739-766 (2000), Table 1). Antibodies with substitutions in an Fc region thereof and increased serum half-lives are also described in WO00/42072, WO 02/060919; Shields et al., J. Biol. Chem. 276:6591-6604 (2001); Hinton, J. Biol. Chem. 279:6213-6216 (2004)). In another embodiment, the serum half-life can also be increased, for example, by attaching other polypeptide sequences. For example, antibodies or other polypeptides useful in the methods of the invention can be attached to serum albumin or a portion of serum albumin that binds to the FcRn receptor or a serum albumin binding peptide so that serum albumin binds to the antibody or polypeptide, e.g., such polypeptide sequences are disclosed in WO01/45746. In one embodiment, the serum albumin peptide to be attached comprises an amino acid sequence of DICLPRWGCLW (SEQ ID NO:3). In another embodiment, the half-life of a Fab is increased by these methods. See also, Dennis et al. J. Biol. Chem. 277:35035-35043 (2002) for additional serum albumin binding peptide sequences.

Engineered antibodies specific for DEspR or VEGFsp with three or more (preferably four) different binding sites are also contemplated (US Appin No. US2002/0004587 A1, Miller et al.).

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

### Anti-Angiogenic Therapeutics and Treatments in General

In some aspects, provided herein are methods and compositions for use in inhibiting angiogenesis in a subject having a disease or disorder dependent or modulated by angiogenesis. Angiogenesis is a process of tissue vascularization that involves the growth of new blood vessels from existing vessels. Tumor vascularization involves angiogenesis, vasculogenesis (de novo development of blood vessels from precursor cells) and co-opting of existing blood vessels. Blood vessels are the means by which oxygen and nutrients are supplied to living tissues and waste products are removed from living tissue. Angiogenesis can be a critical biological process. For example, angiogenesis is essential in reproduction, development and wound repair. Conversely, inappropriate angiogenesis can have severe negative consequences. For example, it is only after solid tumors are vascularized as a result of angiogenesis that the tumors have a sufficient supply of oxygen and nutrients that permit it to grow rapidly and metastasize.

Where the growth of new blood vessels is the cause of, or contributes to, the pathology associated with a disease, inhibition of angiogenesis, using the compositions and methods described herein, can reduce the deleterious effects of the disease. Non-limiting examples include tumors, carotid artery disease, rheumatoid arthritis, diabetic retinopathy, inflammatory diseases, restenosis, and the like. Where the growth of new blood vessels is required to support growth of a deleterious tissue, inhibition of angiogenesis, using the compositions and methods described herein, can reduce the blood supply to the tissue and thereby contribute to reduction in tissue mass based on blood supply requirements. Non-limiting examples include growth of tumors where neovascularization is a continual requirement in order that the tumor growth beyond a few millimeters in thickness, and for the establishment of solid tumor metastases. Another example is coronary plaque enlargement.

There are a variety of diseases or disorders in which angiogenesis is believed to lead to negative consequences, referred to as pathological angiogenesis, or diseases or disorders dependent or modulated by angiogenesis, including but not limited to, inflammatory disorders such as immune and non-immune inflammation, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma and the like cancers which require neovascularization to support tumor growth. In a preferred embodiment of the aspects described herein, the methods are directed to inhibiting angiogenesis in a subject with cancer.

Angiogenesis-dependent diseases and disorders that can be treated using the methods and compositions described herein, are those diseases and disorders affected by vascular growth. In other words, an “angiogenesis-dependent disease or disorder” refers to those diseases or disorders that are dependent on a rich blood supply and blood vessel proliferation for the diseases' pathological progression (e.g., metastatic tumors), or diseases or disorders that are the direct result of aberrant blood vessel proliferation (e.g., diabetic retinopathy and hemangiomas). Non-limiting examples of angiogenesis-dependent diseases or disorder that can be treated using the compositions and methods described herein include abnormal vascular proliferation, ascites formation, psoriasis, age-related macular degeneration, thyroid hyperplasia, preeclampsia, rheumatoid arthritis and osteoarthritis, carotid artery disease, vaso vasorum neovascularization, vulnerable plaque neovascularization, neurodegenerative disorders, Alzheimer's disease, obesity, pleural effusion, atherosclerosis, endometriosis, diabetic/other retinopathies, ocular neovascularizations such as neovascular glaucoma and corneal neovascularization, disorders associated with inappropriate or inopportune invasion of vessels such as diabetic retinopathy, macular degeneration, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma, cancers which require neovascularization to support tumor growth, etc.

Accordingly, described herein are methods of inhibiting angiogenesis in a tissue of a subject or individual having a disease or disorder dependent or modulated by angiogenesis, where the disease or disorder can be treated by the inhibition of angiogenesis. Generally, the methods comprise administering to the subject a therapuetically effective amount of a composition comprising an angiogenesis-inhibiting amount of a DEspR antagonist, such as an anti-VEGFsp antibody or fragment thereof, or a DEspR agonist coupled to an agent that would inhibit the growth of the neovasculature. In some embodiments, the methods further comprise selecting or diagnosing a subject having or at risk for a disease or disorder modulated by angiogenesis.

In other aspects, the compositions and methods described herein are used in blocking or inhibiting angiogenesis that occurs in age-related macular degeneration. It is known, for example, that VEGF contributes to abnormal blood vessel growth from the choroid layer of the eye into the retina, similar to what occurs during the wet or neovascular form of age-related macular degeneration. Macular degeneration, often called AMD or ARMD (age-related macular degeneration), is the leading cause of vision loss and blindness in Americans aged 65 and older. New blood vessels grow (neovascularization) beneath the retina and leak blood and fluid. This leakage causes permanent damage to light-sensitive retinal cells, which die off and create blind spots in central vision or the macula. Accordingly, encompassed in the methods disclosed herein are subjects treated for age-related macular degeneration with anti-angiogenic therapy.

In other aspects, the compositions and methods described herein are used in blocking or inhibiting angiogenesis that occurs in a subject having diabetic retinopathy, where abnormal blood vessel growth is associated with diabetic eye diseases and diabetic macular edema. When normal blood vessels in the retina are damaged by tiny blood clots due to diabetes, a chain reaction is ignited that culminates in new blood vessel growth. However, the backup blood vessels are faulty; they leak (causing edema), bleed and encourage scar tissue that detaches the retina, resulting in severe loss of vision. Such growth is the hallmark of diabetic retinopathy, the leading cause of blindness among young people in developed countries. Therefore, encompassed in the methods disclosed herein are subjects treated for diabetic retinopathy and/or diabetic macular edema.

In other aspects, the compositions and methods described herein are used in blocking or inhibiting angiogenesis that occurs in a subject having rheumatoid arthritis. Rheumatoid arthritis (RA) is characterized by synovial tissue swelling, leukocyte ingress and angiogenesis, or new blood vessel growth. The expansion of the synovial lining of joints in rheumatoid arthritis (RA) and the subsequent invasion by the pannus of underlying cartilage and bone necessitate an increase in the vascular supply to the synovium, to cope with the increased requirement for oxygen and nutrients. Angiogenesis is now recognized as a key event in the formation and maintenance of the pannus in RA (Paleolog, E. M., Arthritis Res. 2002; 4 Suppl 3:S81-90; Afuwape A O, Histol Histopathol. 2002; 17(3):961-72). Even in early RA, some of the earliest histological observations are blood vessels. A mononuclear infiltrate characterizes the synovial tissue along with a luxuriant vasculature. Angiogenesis is integral to formation of the inflammatory pannus and without angiogenesis, leukocyte ingress could not occur (Koch, A. E., Ann. Rheum. Dis. 2000, 59 Suppl 1:165-71). Disruption of the formation of new blood vessels would not only prevent delivery of nutrients to the inflammatory site, it could also reduce joint swelling due to the additional activity of VEGF, a potent proangiogenic factor in RA, as a vascular permeability factor. Anti-VEGF hexapeptide RRKRRR (dRK6) can suppress and mitigate the arthritis severity (Seung-Ah Yoo, et. al., 2005, supra). Accordingly, encompassed in the methods disclosed herein are subjects having or being treated for rheumatoid arthritis.

In other aspects, the compositions and methods described herein are used in blocking or inhibiting angiogenesis that occurs in Alzheimer's disease. Alzheimer's disease (AD) is the most common cause of dementia worldwide. AD is characterized by an excessive cerebral amyloid deposition leading to degeneration of neurons and eventually to dementia. The exact cause of AD is still unknown. It has been shown by epidemiological studies that long-term use of non-steroidal anti-inflammatory drugs, statins, histamine H2-receptor blockers, or calcium-channel blockers, all of which are cardiovascular drugs with anti-angiogenic effects, seem to prevent Alzheimer's disease and/or influence the outcome of AD patients. Therefore, AD angiogenesis in the brain vasculature can play an important role in AD. In Alzheimer's disease, the brain endothelium secretes the precursor substrate for the beta-amyloid plaque and a neurotoxic peptide that selectively kills cortical neurons. Moreover, amyloid deposition in the vasculature leads to endothelial cell apoptosis and endothelial cell activation which leads to neovascularization. Vessel formation could be blocked by the VEGF antagonist SU 4312 as well as by statins, indicating that anti-angiogenesis strategies can interfere with endothelial cell activation in AD (Schultheiss C., el. al., 2006; Grammas P., et. al., 1999) and can be used for preventing and/or treating AD. Accordingly, encompassed in the methods disclosed herein are subjects being treated for Alzheimer's disease.

In other aspects, the compositions and methods described herein are used in blocking or inhibiting angiogenesis that occurs in ischemic regions in the brain, which can contribute to edema, leaky neovessels, and predispose a subject to hemorrhagic transformation after an ischemic stroke event, thus worsening the morbidity and mortality risk from the stroke event. Inhibition of leaky angiogenic neovessels using the compositions and methods described herein can reduce neurologic deficits from an ischemic stroke event, as well as prevent the progression to hemorrhagic stroke. Currently, there is no therapy for ischemic hemorrhagic transformation, nor effective therapies to reduce the neurologic deficits from stroke.

In other aspects, the compositions and methods described herein are used in blocking or inhibiting angiogenesis that occurs in obesity. Adipogenesis in obesity involves interplay between differentiating adipocytes, stromal cells, and blood vessels. Close spatial and temporal interrelationships between blood vessel formation and adipogenesis, and the sprouting of new blood vessels from preexisting vasculature was coupled to adipocyte differentiation. Adipogenic/angiogenic cell clusters can morphologically and immunohistochemically be distinguished from crown-like structures frequently seen in the late stages of adipose tissue obesity. Administration of anti-vascular endothelial growth factor (VEGF) antibodies inhibited not only angiogenesis but also the formation of adipogenic/angiogenic cell clusters, indicating that the coupling of adipogenesis and angiogenesis is essential for differentiation of adipocytes in obesity and that VEGF is a key mediator of that process. (Satoshi Nishimura et. al., 2007, Diabetes 56:1517-1526). It has been shown that the angiogenesis inhibitor, TNP-470 was able to prevent diet-induced and genetic obesity in mice (Ebba Bråkenhielm et. al., Circulation Research, 2004; 94:1579). TNP-470 reduced vascularity in the adipose tissue, thereby inhibiting the rate of growth of the adipose tissue and obesity development. Accordingly, encompassed in the methods disclosed herein are subjects suffering from obesity.

In other aspects, the compositions and methods described herein are used in blocking or inhibiting angiogenesis that occurs in endometriosis. Excessive endometrial angiogenesis is proposed as an important mechanism in the pathogenesis of endometriosis (Healy, D L., et. al., Hum Reprod Update. 1998 September-October; 4(5):736-40). The endometrium of patients with endometriosis shows enhanced endothelial cell proliferation. Moreover there is an elevated expression of the cell adhesion molecule integrin vB3 in more blood vessels in the endometrium of women with endometriosis when compared with normal women. The U.S. Pat. No. 6,121,230 described the use of anti-VEGF agents in the treatment of endometriosis and this patent is hereby incorporated by reference. Accordingly, encompassed in the methods disclosed herein are subjects having or being treated for endometriosis.

As described herein, any of a variety of tissues, or organs comprised of organized tissues, can support angiogenesis in disease conditions including skin, muscle, gut, connective tissue, joints, bones and the like tissue in which blood vessels can invade upon angiogenic stimuli. In some embodiments of these aspects, the given parameter or phenotype to be inhibited can include, but is not limited to, the mean total tube number in an in vitro tubulogenesis assay, the mean total tube length in an in vitro tubulogenesis assay, the mean number of branching points in an in vitro tubulogenesis assay, the mean number of vessel connections in an in vitro tubulogenesis assay, and tumor cell invasiveness. In some aspects, the disease or disorder dependent or modulated by angiogenesis is cancer, where the rapidly dividing neoplastic cancer cells require an efficient blood supply to sustain their continual growth of the tumor. Inhibition of angiogenesis or tumor cell invasiveness or a combination thereof using the compositions and therapeutic methods described herein at the primary tumor site and secondary tumor site serve to prevent and limit metastasis and progression of disease.

In some embodiments, the methods can further comprise first selecting or diagnosing the subject having or at risk for a disease or disorder, such as a cancer or tumor. In some such embodiments, the diagnosis of the subject can comprise administering to the subject an anti-DEspR agonist antibody or antibody fragment thereof coupled to a label, for example, a radioactive label, or a label used for molecular imaging, as described elsewhere herein. In such embodiments, detection of the labeled anti-DEspR agonist antibody or antibody fragment is indicative of the subject having a cancer or tumor. Examples of cancer include but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; glioblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

The cells treated according to the inventions described herein, whether they be cells that are endothelial cells or cancer cells, are ones expressing DEspR.

### Atherosclerotic Plaque

In other aspects, the compositions described herein are used in the treatment or inhibition or imaging of artherosclerotic plaques and atherosclerosis. Such compositions are anti-VEGFsp antibodies and DEspR binding fragments thereof. Atherosclerosis is the most common form of vascular disease and is a disorder of large arteries that underlies most coronary artery disease, aortic aneurysm, cerebrovascular disease and arterial disease of lower extremities (Libby, in “The Principles of Internal Medicine”, 15th ed., Braunward et al. (editors), Saunders, Philadelphia, Pa., 2001, pp. 1377-1382.). The pathogenesis of atherosclerosis occurs as a reaction to injury (Libby, in “The Principles of Internal Medicine”, 15th ed., Braunwald et al. (editors), Saunders, Philadelphia, Pa., 2001, pp. 1377-1382.). The injury to the endothelium can be subtle, resulting in a loss of the ability of the cells to function normally. Examples of types of injury to the endothelium include hypercholesterolemia and mechanical stress (Ross, 1999, N. Engl. J. Med., 340:115).

The process of atherosclerosis involves inflammation, and white blood cells (e.g., lymphocytes, monocytes, and macrophages) are often present throughout the development of atherosclerosis. Atherosclerosis begins when monocytes are activated and move out of the bloodstream into the wall of an artery. There, they are transformed into foam cells, which collect cholesterol and other fatty materials. In time, these fat-laden foam cells accumulate and form atheromas in the lining of the artery's wall, causing a thickening and hardening of the wall. Atheromas can be scattered throughout medium-sized and large arteries, but usually form where the arteries branch. Treatment of and diagnosis of atherosclerosis is important because it often leads to heart disease and can also cause stroke or other vascular problems such as claudication.

Accordingly, in some embodiments of the aspects described herein, pathological angiogenesis in atherosclerotic plaques and in the vasa vasorum of atherosclerotic arteries (coronary and carotid artery disease) is considered a risk and/or causal factor for vulnerable plaque progression and disruption. Thus, in some such embodiments, a subject having an angiogenic disorder to be treated using the compositions and methods described herein can have or be at risk for atherosclerosis. As used herein, “atherosclerosis” refers to a disease of the arterial blood vessels resulting in the hardening of arteries caused by the formation of multiple atheromatous plaques within the arteries. Atherosclerosis can be associated with other disease conditions, including but not limited to, coronary heart disease events, cerebrovascular events, acute coronary syndrome, and intermittent claudication. For example, atherosclerosis of the coronary arteries commonly causes coronary artery disease, myocardial infarction, coronary thrombosis, and angina pectoris. Atherosclerosis of the arteries supplying the central nervous system frequently provokes strokes and transient cerebral ischemia. In the peripheral circulation, atherosclerosis causes intermittent claudication and gangrene and can jeopardize limb viability. Atherosclerosis of an artery of the splanchnic circulation can cause mesenteric ischemia. Atherosclerosis can also affect the kidneys directly (e.g., renal artery stenosis). Also, persons who have previously experienced one or more non-fatal atherosclerotic disease events are those for whom the potential for recurrence of such an event exists.

Sometimes these other diseases can be caused by or associated with other than atherosclerosis. Therefore, in some embodiments, one first diagnoses that atherosclerosis is present prior to administering the compositions described herein to the subject.A subject is “diagnosed with atherosclerosis” or “selected as having atherosclerosis” if at least one of the markers of symptoms of atherosclerosis is present. In one such embodiment, the subject is “selected” if the person has a family history of atherosclerosis or carries a known genetic mutation or polymorphism for high cholesterol. In one embodiment, a subject is diagnosed by measuring an increase level of C-reactive protein (CRP) in the absence of other inflammatory disorders. In other embodiments, atherosclerosis is diagnosed by measuring serum levels of homocysteine, fibrinogen, lipoprotein (a), or small LDL particles. Alternatively a computed tomography scan, which measures calcium levels in the coronary arteries, can be used to select a subject having atherosclerosis. In one embodiment, atherosclerosis is diagnosed by an increase in inflammatory cytokines. In one embodiment, increased interleukin-6 levels is used as an indicator to select an individual having atherosclerosis. In other embodiments, increased interleukin-8 and/or interleukin-17 level is used as an indicator to select an individual having atherosclerosis.

### Subjects

The individual or subject to be treated as described herein in various embodiments is desirably a human patient, although it is to be understood that the methods are effective with respect to all mammals, which are intended to be included in the term “patient” or “subject”. In this context, a mammal is understood to include any mammalian species in which treatment of diseases associated with angiogenesis is desirable. The terms “subject” and “individual” are used interchangeably herein, and refer to an animal, for example a human, recipient of the DEspR-specific treatments. For treatment of disease states which are specific for a specific animal such as a human subject, the term “subject” refers to that specific animal. The terms “non-human animals” and “non-human mammals” are used interchangeably herein, and include mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like are also encompassed in the term subject.

### Modes of Administration

The DEspR-specific antagonist agents, and the DEspR-specific agonist conjugates described herein can be administered to a subject in need thereof by any appropriate route which results in an effective treatment in the subject. As used herein, the terms “administering,” and “introducing” are used interchangeably and refer to the placement of a therapeutic into a subject by a method or route which results in at least partial localization of such agents at a desired site.

In some embodiments, the agent is administered to a subject by any mode of administration that delivers the agent systemically or to a desired surface or target, and can include, but is not limited to, injection, infusion, instillation, and inhalation administration. To the extent that the agent can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the anti-DEspR antibodies or antibody fragments thereof for use in the methods described herein are administered by intravenous infusion or injection.

The phrases “parenteral administration” and “administered parenterally” refer to modes of administration other than enteral and topical administration, usually by injection. The phrases “systemic administration,” and “administered systemically”, refer to the administration of the agent other than directly into a target site, tissue, or organ, such as a tumor site, such that it enters the subject's circulatory system and, thus, is subject to metabolism and other like processes.

The various therapeutic agents described herein are administered to a subject, e.g., a human subject, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Local administration, for example, to a tumor or cancer site where angiogenesis is occurring, is particularly desired if extensive side effects or toxicity is associated with the use of the agent. An ex vivo strategy can also be used for therapeutic applications in some embodiments.

In some embodiments, when the therapeutic agent is an antibody or antibody fragment thereof, the antibody or antibody fragment thereof is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In some embodiments, the antibody or antibody fragment thereof is suitably administered by pulse infusion, particularly with declining doses of the antibody. Preferably the dosing is given by injections, most preferably intravenous or subcutaneous injections, depending in part on whether the administration is brief or chronic.

In some embodiments, the therapeutic agent is administered locally, e.g., by direct injections, when the disorder or location of the tumor permits, and the injections can be repeated periodically. The agent can also be delivered systemically to the subject.

### Pharmaceutical Formulations

For the clinical use of the methods described herein, administration of the DEspR antagonists or agonists, such as the anti-VEGFsp antibodies or antibody fragments thereof, VEGFsp or fragments thereof described herein, can include formulation into pharmaceutical compositions or pharmaceutical formulations for parenteral administration, e.g., intravenous; mucosal, e.g., intranasal; ocular, or other mode of administration. In some embodiments, the anti VEGFsp antibodies or antibody fragments thereof described herein can be administered along with any pharmaceutically acceptable carrier compound, material, or composition which results in an effective treatment in the subject. Thus, a pharmaceutical formulation for use in the methods described herein can contain an anti-VEGFsp antibody or antibody fragment thereof as described herein in combination with one or more pharmaceutically acceptable ingredients.

In some aspects, provided herein are pharmaceutical preparations comprising a human or humanized antibody or fragment thereof that binds selectively to VEGFsp and a pharmaceutically acceptable carrier. In some embodiments, the anti-VEGFsp antibodies or antibody fragments thereof described herein can be administered along with any pharmaceutically acceptable carrier compound, material, or composition which results in an effective treatment in the subject. The antibody or fragment thereof may be a monoclonal antibody, and in some embodiments, the antibody or fragment thereof blocks binding of VEGFsp to DEspR. The antibody may also have an Fc region modified to promote clearance from circulation of the antibody. In some aspects, provided herein are compositions comprising VEGFsp or a fragment thereof that binds DEspR conjugated to a toxin. Also provided herein are pharmaceutical preparations comprising VEGFsp or a fragment thereof that binds DEspR conjugated to a toxin and a pharmaceutically acceptable carrier. In some such embodiments, the VEGFsp is human VEGFsp. In some such embodiments, the VEGFsp target has a sequence comprising SEQ ID NO:2 or an allelic variant thereof. In some embodiments, the VEGFsp or the fragment thereof that binds DEspR is coupled to a particle that is coupled to, coated with, embedded with or contains the toxin. In some embodiments, the particle is a solid polymer matrix. The VEGFsp or fragment thereof can be covalently or non-covalently conjugated to the toxin.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an anti-DEspR antibody or antibody fragment thereof. Each carrier must be “acceptable” in the sense of being compatible with the other ingredients of the formulation and not injurious to the patient. The terms “excipient”, “carrier”, “pharmaceutically acceptable carrier” or the like are used interchangeably herein.

The anti-VEGFsp antibodies or antibody fragments thereof described herein can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (79) nasally. Additionally, an anti-DEspR antibody or antibody fragment thereof can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

Therapeutic formulations of the DEspR-specific antagonist and agonist agents, such as anti-DEspR antibodies or antibody fragments thereof, described herein can be prepared for storage by mixing a DEspR-specific antagonist having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEENT™, PLURONICS™ or polyethylene glycol (PEG). Exemplary lyophilized anti-VEGF antibody formulations are described in WO 97/04801, expressly incorporated herein be reference.

Optionally, but preferably, the formulations comprising the compositions described herein contain a pharmaceutically acceptable salt, typically, e.g., sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are examples of preservatives. Optionally, the formulations of the invention can include a pharmaceutically acceptable surfactant at a concentration of 0.005 to 0.02%.

The therapeutic formulations of the compositions comprising DEspR-specific antagonists, such as anti-DEspR antibodies and antibody fragments thereof, described herein can also contain more than one active compound as necessary for the particular indication being treated, preferably those with complementary activities that do not adversely affect each other. For example, in some embodiments, it can be desirable to further provide antibodies which bind to EGFR, VEGF (e.g. an antibody which binds a different epitope on VEGF), VEGFR, or ErbB2 (e.g., HERCEPTIN™). Alternatively, or in addition, the composition can comprise a cytotoxic agent, cytokine, growth inhibitory agent and/or VEGFR antagonist. Such molecules are suitably present in combination in amounts that are effective for the purpose intended.

The active ingredients of the therapeutic formulations of the compositions comprising DEspR-specific antagonists described herein can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In some embodiments, sustained-release preparations can be used. Suitable examples of sustained-release preparations include semipermeable matrices of solid hydrophobic polymers containing the DEspR-specific antagonist, such as an anti-DEspR antibody, in which the matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

The therapeutic formulations to be used for in vivo administration, such as parenteral administration, in the methods described herein can be sterile, which is readily accomplished by filtration through sterile filtration membranes, or other methods known to those of skill in the art.

### Dosages and Duration

The DEspR-specific antagonists described herein, such as anti-VEGFsp antibodies and antibody fragments thereof, and the DEspR agonist-conjugates, are formulated, dosed, and administered in a fashion consistent with good medical practice. Factors for consideration in this context include the particular disorder being treated, the particular subject being treated, the clinical condition of the individual subject, the cause of the disorder, the site of delivery of the agent, the method of administration, the scheduling of administration, and other factors known to medical practitioners. In the treatment of cancer, the “therapeutically effective amount” to be administered will be governed by such considerations, and refers to the minimum amount necessary to ameliorate, treat, or stabilize, the cancer; to increase the time until progression (duration of progression free survival) or to treat or prevent the occurrence or recurrence of a tumor, a dormant tumor, or a micrometastases. The therapeutic agent is optionally formulated with one or more additional therapeutic agents currently used to prevent or treat cancer or a risk of developing a cancer. The effective amount of such other agents depends on the amount of VEGFsp-specific antibody or DEspR binding fragment thereof, or DEspR agonist-conjugate present in the formulation, the type of disorder or treatment, and other factors discussed above. These are generally used in the same dosages and with administration routes as used herein before or about from 1 to 99% of the heretofore employed dosages.

Depending on the type and severity of the disease, about 1 μg/kg to 100 mg/kg (e.g., 0.1-20 mg/kg) of a therapeutic agent is an initial candidate dosage for administration to a subject, whether, for example, by one or more separate administrations, or by continuous infusion. A typical daily dosage might range from about 1 μg/kg to about 100 mg/kg or more, depending on the factors mentioned above. Particularly desirable dosages include, for example, 5 mg/kg, 7.5 mg/kg, 10 mg/kg, and 15 mg/kg. For repeated administrations over several days or longer, depending on the condition, the treatment is sustained until, for example, the cancer is treated, as measured by the methods described above or known in the art. However, other dosage regimens can be useful. In one non-limiting example, if the VEGFsp-specific antibody or fragment thereof is administered once every week, every two weeks, or every three weeks, at a dose range from about 5 mg/kg to about 15 mg/kg, including but not limited to 5 mg/kg, 7.5 mg/kg, 10 mg/kg or 15 mg/kg. The progress of using the methods described herein can be easily monitored by conventional techniques and assays.

When administered to a subject, effective amounts of the selected therapeutic agent will depend, of course, on the particular disease or condition being treated; the severity of the disease or condition; individual patient parameters including age, physical condition, size and weight, concurrent treatment, frequency of treatment, and the mode of administration. These factors are well known to those of ordinary skill in the art and can be addressed with no more than routine experimentation. In some embodiments, a maximum dose is used, that is, the highest safe dose according to sound medical judgment. The duration of a therapy using the methods described herein will continue for as long as medically indicated or until a desired therapeutic effect (e.g., those described herein) is achieved. In embodiments, the therapy is continued for 1 month, 2 months, 4 months, 6 months, 8 months, 10 months, 1 year, 2 years, 3 years, 4 years, 5 years, 10 years, 20 years, or for a period of years up to the lifetime of the subject.

### Efficacy of the Treatment

The DEspR inhibitors are administered in effective amounts. An effective amount is a dose sufficient to provide a medically desirable result and can be determined by one of skill in the art using routine methods. In the treatment of stroke, an effective amount will be that amount necessary to inhibit edema, leaky neovessels, and/or predispose a subject to hemorrhagic transformation after an ischemic stroke event, thus worsening the morbidity and mortality risk from the stroke event. Inhibition of leaky angiogenic neovessels using the compositions and methods described herein can reduce neurologic deficits from an ischemic stroke event, as well as prevent the progression to hemorrhagic stroke. In some embodiments, an effective amount is an amount effective to inhibit micro-hemorrhages. In some embodiments, an effective amount is an amount effective to inhibit neurological deficit in the subject. In some embodiments, an effective amount is an amount which results in any improvement in the condition being treated. In some embodiments, an effective amount may depend on the use of one or more additional therapeutic agents, where combination therapy is contemplated. However, one of skill in the art can determine appropriate doses and ranges of DEspR inhibitors to use, for example based on in vitro and/or in vivo testing and/or other knowledge of compound dosages.

The efficacy of the treatment methods for cancer comprising therapeutic formulations of the compositions described herein can be measured by various endpoints commonly used in evaluating cancer treatments, including but not limited to, tumor regression, tumor weight or size shrinkage, time to progression, duration of survival, progression free survival, overall response rate, duration of response, and quality of life. Because the certain therapeutic described herein target the tumor vasculature, cancer cells, and some cancer stem cell subsets, they represent a unique class of multi-targeting anticancer drugs, and therefore can require unique measures and definitions of clinical responses to drugs. For example, tumor shrinkage of greater than 50% in a 2-dimensional analysis is the standard cut-off for declaring a response. However, the therapeutic agents that target tumor vasculature, cancer cells, and some cancer stem cell subsets, described herein, can cause inhibition of metastatic spread without shrinkage of the primary tumor, or can simply exert a tumoristatic effect. Accordingly, novel approaches to determining efficacy of an anti-angiogenic therapy should be employed, including for example, measurement of plasma or urinary markers of angiogenesis, and measurement of response through molecular imaging, using, for example, a DEspR-antibody or antibody fragment conjugated to a label, such as a microbubble. In the case of cancers, the therapeutically effective amount can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the therapeutic agent can prevent growth and/or kill existing cancer cells, it can be cytostatic and/or cytotoxic. For cancer therapy, efficacy in vivo can, for example, be measured by assessing the duration of survival, duration of progression free survival (PFS), the response rates (RR), duration of response, and/or quality of life.

In other embodiments, described herein are methods for increasing progression free survival of a human subject susceptible to or diagnosed with a cancer. Time to disease progression is defined as the time from administration of the drug until disease progression or death. In an embodiment, the treatment of the invention using a DEspR-specific inhibitor, such as an anti-VEGFsp antibody or antibody fragment thereof, or a DEspR agonist conjugated to a toxin significantly increases progression free survival by at least about 1 month, 1.2 months, 2 months, 2.4 months, 2.9 months, 3.5 months, preferably by about 1 to about 5 months. In some cases, the survival is increased between 1 and 5 months.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with a chronic immune condition, such as, but not limited to, a chronic infection or a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

A patient who is being treated for a cancer or stroke or any other condition described herein is one who a medical practitioner has diagnosed as having such a condition. Diagnosis can be by any suitable means. Diagnosis and monitoring can involve, for example, detecting the level of cells in a biological sample (for example, a tissue or lymph node biopsy, blood test, or urine test), detecting the level of a surrogate marker in a biological sample, or detecting symptoms associated with the specific disorder.

The term “effective amount” as used herein refers to the amount of an anti-DEspR antibody or antibody fragment thereof needed to alleviate at least one or more symptom of the disease or disorder. An effective amount as used herein would also include an amount sufficient to delay the development of a symptom of the disease, alter the course of a symptom disease (for example but not limited to, slow the progression of a symptom of the disease), or reverse a symptom of the disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dosage can vary depending upon the dosage form employed and the route of administration utilized. The dose ratio between toxic and therapeutic effects is the therapeutic index and can be expressed as the ratio LD₅₀/ED₅₀. Compositions and methods that exhibit large therapeutic indices are preferred. A therapeutically effective dose can be estimated initially from cell culture assays. Also, a dose can be formulated in animal models to achieve a circulating plasma concentration range that includes the IC₅₀ (i.e., the concentration of the anti-DEspR antibody or antibody fragment thereof), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

### Combination Antiangiogenic Therapies

In other embodiments, the methods provided for inhibiting angiogenesis in a tissue of a subject or individual having a disease or disorder dependent or modulated by angiogenesis by administering to the subject a therapuetically effective amount of a composition comprising an angiogenesis-inhibiting amount of an DEspR inhibitor, such as an anti-VEGFsp antibody or antibody fragment thereof, can further comprise administration one or more additional treatments such as angiogenic inhibitors, chemotherapy, radiation, surgery, or other treatments known to those of skill in the art to inhibit angiogenesis. An exemplary and non-limiting list of chemotherapeutic agents contemplated for use in the methods described herein is provided under “Definition,” or described herein.

As used herein, combined administration includes simultaneous administration, using separate formulations or a single pharmaceutical formulation, and consecutive administration in either order, wherein preferably there is a time period while both (or all) active agents simultaneously exert their biological activities. Preparation and dosing schedules for such chemotherapeutic agents can be used according to manufacturers' instructions or as determined empirically by the skilled practitioner. Preparation and dosing schedules for chemotherapy are also described in Chemotherapy Service Ed., M. C. Perry, Williams & Wilkins, Baltimore, Md. (1992). Accordingly, in some embodiments, the chemotherapeutic agent can precede, or follow administration of the DEspR-specific antagonist or can be given simultaneously therewith.

In some other embodiments of the methods described herein, other therapeutic agents useful for combination tumor therapy with therapeutic agents of the invention include antagonists of other factors that are involved in tumor growth, such as EGFR, ErbB2 (also known as Her2), ErbB3, ErbB4, or TNF. In some embodiments, it can be beneficial to also administer one or more cytokines to the subject. In some embodiments, the DEspR antagonist is co-administered with a growth inhibitory agent. For example, the growth inhibitory agent can be administered first, followed by the DEspR antagonist. However, simultaneous administration or administration of the DEspR antagonist first is also contemplated. Suitable dosages for the growth inhibitory agent are those presently used and can be lowered due to the combined action (synergy) of the growth inhibitory agent and therapeutic agent of the invention.

Examples of additional angiogenic inhibitors that can be used in combination with the therapeutic agents of the invention described herein include, but are not limited to: direct angiogenesis inhibitors, Angiostatin, Bevacizumab (AVASTIN®), Arresten, Canstatin, Combretastatin, Endostatin, NM-3, Thrombospondin, Tumstatin, 2-methoxyestradiol, cetuximab (ERBITUX®), panitumumab (VECTIBIX™), trastuzumab (HERCEPTIN®) and Vitaxin; and indirect angiogenesis inhibitors: ZD1839 (IRESSA™), ZD6474, OS1774 (TARCEVA), CI1033, PKI1666, IMC225 (ERBITUX®), PTK787, SU6668, SU11248, HERCEPTIN, and IFN-α, CELEBREX® (Celecoxib), THALOMID® (Thalidomide), and IFN-α.

In some embodiments, the additional angiogenesis inhibitors for use in the methods described herein include but are not limited to small molecule tyrosine kinase inhibitors (TKIs) of multiple pro-angiogenic growth factor receptors. The three TKIs that are currently approved as anti-cancer therapies are erlotinib (TARCEVA®), sorafenib (NEXAVAR®), and sunitinib (SUTENT®).

In some embodiments, the angiogenesis inhibitors for use in the methods described herein include but are not limited to inhibitors of mTOR (mammalian target of rapamycin) such as temsirolimus (TORICEL™), bortezomib (VELCADE®), thalidomide (THALOMID®), and Doxycyclin,

In other embodiments, the angiogenesis inhibitors for use in the methods described herein include one or more drugs that target the VEGF pathway (as opposed to the VEGFsp pathway). Bevacizumab (AVASTIN®) was the first drug that targeted new blood vessels to be approved for use against cancer. It is a monoclonal antibody that binds to VEGF, thereby blocking VEGF from reaching the VEGF receptor (VEGFR). Other drugs, such as sunitinib (SUTENT®) and sorafenib (NEXAVAR®), are small molecules that attach to the VEGF receptor itself, preventing it from being turned on. Such drugs are collectively termed VEGF inhibitors. As the VEGF/VPF protein interacts with the VEGFRs, inhibition of either the ligand VEGF, e.g. by reducing the amount that is available to interact with the receptor; or inhibition of the receptor's intrinsic tyrosine kinase activity, blocks the function of this pathway. This pathway controls endothelial cell growth, as well as permeability, and these functions are mediated through the VEGFRs.

As will be understood by those of ordinary skill in the art, the appropriate doses of chemotherapeutic agents or other anti-cancer agents will be generally around those already employed in clinical therapies, e.g., where the chemotherapeutics are administered alone or in combination with other chemotherapeutics. Variation in dosage will likely occur depending on the condition being treated. The physician administering treatment will be able to determine the appropriate dose for the individual subject.

In addition to the above therapeutic regimes, the subject can be subjected to radiation therapy.

Some aspects and embodiments disclosed herein can be illustrated by, for example any of the following numbered paragraphs:
1. A method of treatment comprising administering to a human subject within two days of the subject having a stroke a DEspR inhibitor in an amount effective to treat the stroke.
2. The method of paragraph 1, wherein the stroke is an ischemic stroke.
3. The method of paragraph 1, wherein the stroke is a hemorrhagic stroke.
4. The method of paragraph 1, wherein the amount is effective to inhibit micro-hemorrhages.
5. The method of paragraph 1, wherein the subject has had surgery for a hemorrhagic stroke.
6. The method of paragraph 1, wherein the amount is effective to inhibit neurological deficit in the subject.
7. The method of paragraph 1, wherein the DEspR inhibitor is administered to the subject within one day of the subject having the stroke.
   8. The method of paragraph 1, wherein the DEspR inhibitor is administered to the subject within 12 hours of the subject having the stroke.
9. The method of paragraph 1, wherein the DEspR inhibitor is administered to the subject (i) within four hours, (ii) within two hours, or (iii) within one hour of the subject having the stroke.
10. The method of any one of paragraphs 1-9, wherein the DEspR inhibitor comprises a monoclonal antibody or fragment thereof that binds DEspR or VEGFsp.
11. The method of any one of paragraphs 1-9, wherein the DEspR inhibitor comprises a human or humanized monoclonal antibody or fragment thereof that binds DEspR or VEGFsp.
12. The method of paragraph 11, wherein the human or humanized monoclonal antibody or fragment thereof binds residues 1-9 of SEQ ID No. 1.
13. The method of paragraph 12, wherein the human or humanized monoclonal antibody comprises (i) a heavy chain variable region that is SEQ ID No. 4, (ii) a light chain variable region that is SEQ ID No. 9, or a heavy chain variable region that is SEQ ID No. 4 and a light chain variable region that is SEQ ID No. 9.
14. The method of paragraph 11, wherein the human or humanized monoclonal antibody or fragment thereof binds VEGFsp.
15. A composition for treating stroke comprising a DEspR inhibitor.
16. The composition of paragraph 15, wherein the DEspR inhibitor comprises a monoclonal antibody or fragment thereof that binds DEspR or VEGFsp.
17. The composition of paragraph 15, wherein the DEspR inhibitor comprises a human or humanized monoclonal antibody or fragment thereof that binds DEspR or VEGFsp.
18. The composition of paragraph 15, wherein the human or humanized monoclonal antibody or fragment thereof binds residues 1-9 of SEQ ID No. 1.
19. The composition of paragraph 15, wherein the human or humanized monoclonal antibody comprises (i) a heavy chain variable region that is SEQ ID No. 4, (ii) a light chain variable region that is SEQ ID No. 9, or a heavy chain variable region that is SEQ ID No. 4 and a light chain variable region that is SEQ ID No. 9.
20. The composition of paragraph 15, wherein the human or humanized monoclonal antibody or fragment thereof binds VEGFsp.
21. A method of inhibiting an adverse neurological event comprising administering to a human subject having or suspected of having micro-hemorrhages a DEspR inhibitor in an amount effective to inhibit the adverse neurological event.
22. The method of paragraph 21, wherein the adverse neurological event is further micro-hemorrhages.
23. The method of paragraph 21, wherein the adverse neurological event is recurrent cerebral hemorrhage.
24. The method of paragraph 21, wherein the adverse neurological event is neurological deficit.
25. A method of treating cancer comprising administering to a subject having a cancer expressing DEspR an antibody or fragment thereof that binds selectively to VEGFsp in an amount effective to inhibit the cancer.
26. The method of paragraph 25 wherein the antibody or fragment thereof is a monoclonal antibody.
27. The method of paragraph 25 wherein the antibody or fragment thereof is a human or humanized monoclonal antibody.
28. The method of any one of paragraphs 25-27 wherein the antibody or fragment thereof blocks binding of VEGFsp to DEspR.
29. The method of paragraph 25 wherein the antibody is administered to the subject.
30. The method of paragraph 28 wherein the antibody is administered to the subject.
31. The method of paragraph 30 wherein the antibody has an Fc region modified to promote clearance from circulation of the antibody.
32. The method of paragraph 25 wherein the fragment is administered to the subject.
33. The method of paragraph 28 wherein the fragment is administered to the subject.
34. A method of inhibiting angiogenesis comprising administering to a subject having a disease or disorder dependent on or modulated by angiogenesis, an antibody or fragment thereof that binds selectively VEGFsp in an amount effective to inhibit the angiogenesis.
35. The method of paragraph 34 wherein the disease or disorder is age-related macular degeneration, carotid artery disease, diabetic retinopathy, rheumatoid arthritis, a neurodegenerative disease, Alzheimer's disease, obesity, endometriosis, psoriasis, atherosclerosis, ocular neovascularization, neovascular glaucoma, osteoporosis, or restenosis.
36. The method of paragraph 35 wherein the antibody or fragment thereof is a monoclonal antibody.
37. The method of paragraph 35 wherein the antibody or fragment thereof is a human or humanized monoclonal antibody.
38. The method of any one of paragraphs 35-37 wherein the antibody or fragment thereof blocks binding of VEGFsp to DEspR.
39. The method of any one of paragraphs 35-37 wherein the antibody is administered to the subject.
40. The method of paragraph 38 wherein the antibody is administered to the subject.
41. The method of paragraph 40 wherein the antibody has an Fc region modified to promote clearance from circulation of the antibody.
42. The method of any one of paragraphs 35-37 wherein the fragment is administered to the subject.
43. The method of paragraph 38 wherein the fragment is administered to the subject.
44. A pharmaceutical preparation comprising a human or humanized antibody or fragment thereof that binds selectively VEGFsp and a pharmaceutically acceptable carrier constructed and arranged for administration to a human.
45. The pharmaceutical preparation of paragraph 44 wherein the antibody or fragment thereof is a monoclonal antibody.
46. The pharmaceutical preparation of any one of paragraphs 44-45 wherein the antibody or fragment thereof blocks binding of VEGFsp to DEspR.
47. The pharmaceutical preparation of any one of paragraphs 44-45 wherein the pharmaceutical preparation comprises the antibody.
48. The pharmaceutical preparation of paragraph 47 wherein the antibody has an Fc region modified to promote clearance from circulation of the antibody.
49. The pharmaceutical preparation of paragraph 46 wherein the pharmaceutical preparation comprises the antibody.
50. The pharmaceutical preparation of paragraph 49 wherein the antibody has an Fc region modified to promote clearance from circulation of the antibody.
51. The pharmaceutical preparation of any one of paragraphs 44-45 wherein the pharmaceutical preparation comprises the fragment.
52. The pharmaceutical preparation of paragraph 46 wherein the pharmaceutical preparation comprises the fragment.
53. A composition comprising VEGFsp, or a fragment thereof that binds DEspR, coupled to a toxin.
54. The composition of paragraph 53, wherein the VEGFsp or the fragment thereof that binds DEspR is covalently coupled to a toxin.
55. The composition of paragraph 54, wherein the toxin is a radiotoxin.
56. The composition of paragraph 54, wherein the toxin is a chemotoxin.
57. The composition of paragraph 53, wherein the VEGFsp or the fragment thereof that binds DEspR is non-covalently coupled to a toxin.
58. The composition of paragraph 53, wherein the VEGFsp or the fragment thereof that binds DEspR is coupled to a particle that is coupled to, coated with, embedded with or contains the toxin.
59. The composition of paragraph 58, wherein the particle is a solid polymer matrix.
60. The composition of paragraph 58, wherein the particle is a liposome.
61. The composition of any one of paragraphs 58-60, wherein the toxin is a radiotoxin.
62. The composition of any one of paragraphs 58-60, wherein the toxin is a chemotoxin.
63. A pharmaceutical preparation comprising VEGFsp, or a fragment thereof that binds DEspR, coupled to a pharmaceutical agent, and a pharmaceutically acceptable carrier constructed and arranged for administration to a human.
64. The pharmaceutical preparation of paragraph 63, wherein the VEGFsp or the fragment thereof that binds DEspR is covalently coupled to a toxin.
65. The pharmaceutical preparation of paragraph 64, wherein the toxin is a radiotoxin.
66. The pharmaceutical preparation of paragraph 64, wherein the toxin is a chemotoxin.
67. The pharmaceutical preparation of paragraph 63, wherein the VEGFsp or the fragment thereof that binds DEspR is non-covalently coupled to a toxin.
68. The pharmaceutical preparation of paragraph 63, wherein the VEGFsp or the fragment thereof that binds DEspR is coupled to a particle that is coupled to, coated with, embedded with or contains the toxin.
69. The pharmaceutical preparation of paragraph 68, wherein the particle is a solid polymer matrix.
70. The pharmaceutical preparation of paragraph 68, wherein the particle is a liposome.
71. The pharmaceutical preparation of any one of paragraphs 67-70, wherein the toxin is a radiotoxin.
72. The composition of any one of paragraphs 67-70, wherein the toxin is a chemotoxin.
73. A method for inhibiting growth of tumor cells comprising contacting tumor cells expressing DEspR with a DEspR agonist coupled to a toxin, in an amount effective to inhibit growth of the tumor.
74. The method of paragraph 73 wherein the tumor cells are in a subject who has had one or more of (i) radiation treatment for cancer, (ii) chemotherapy for cancer, or (iii) surgical treatment for cancer.
75. The method of paragraph 73 wherein the DEspR agonist is an antibody or fragment thereof that binds DEspR.
76. The method of paragraph 75 wherein the antibody or fragment thereof is a monoclonal antibody.
77. The method of paragraph 75 wherein the antibody or fragment thereof is a human or humanized monoclonal antibody.
78. The method of any one of paragraphs 75-77 wherein the antibody or fragment thereof blocks binding of VEGFsp to DEspR.
79. The method of paragraph 73 wherein the DEspR agonist is VEGFsp or a fragment of VEGFsp that binds DEspR.
80. The method of any one of paragraphs 75 or 77, wherein the DEspR agonist is covalently coupled to a toxin.
81. The method of paragraph 80, wherein the toxin is a radiotoxin.
82. The method of paragraph 80, wherein the toxin is a chemotoxin.
83. The method of paragraph 80, wherein the wherein the DEspR agonist is non-covalently coupled to the toxin.
84. The method of paragraph 80, wherein the DEspR agonist is coupled to a particle that is coupled to, coated with, embedded with or contains the toxin.
85. The method of paragraph 84, wherein the particle is a solid polymer matrix.
86. The method of paragraph 83, wherein the particle is a liposome.
87. The method of any one of paragraphs 83-86, wherein the toxin is a radiotoxin.
88. The method of any one of paragraphs 83-86, wherein the toxin is a chemotoxin.
89. A method of reducing cancer re-occurrence comprising administering to a subject after the subject has had one or more of (i) radiation treatment for cancer, (ii) surgical treatment for cancer and (iii) chemotherapy treatment for cancer, a DEspR inhibitor in an amount effective to reduce cancer re-occurrence.
90. A method for identifying a circulating tumor cell comprising contacting a circulating tumor cell expressing DEspR with an agent that binds DEspR, and detecting the agent bound to the circulating tumor cell.
91. The method of paragraph 90, wherein the agent is (i) an antibody that binds DEspR or (ii) VEGFsp.
92. The method of paragraph 91, wherein the agent is labeled.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN¹-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), which are all incorporated by reference herein in their entireties.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular. As used in this specification and the appended claims, the singular forms “a,” “an,” and the include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” The term “about” when used in connection with percentages can mean±1%.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

This invention is further illustrated by the following examples which should not be construed as limiting.

### EXAMPLES

### Example 1

### Development of Novel Anti-Human Dual Endothelin-1/VEGFsp Receptor (anti-hDEspR) Monoclonal Antibody Treatments as Inhibitors of Tumor Angiogenesis and Tumor Cell Invasiveness

DEspR is a key angiogenesis player in embryonic development as seen in DEspR^{-/-} knockout mice (Herrera et al. 2005), and contributes to adult tissue vascularity as seen in adult haplo-deficient (+/−) mice exhibiting decreased tissue vascularity shown by power Doppler analysis.

Based on the association of tumor invasion and metastasis with intrinsic and evasive resistance to VEGF-targeted therapies, the combination of anti-invasive and anti-metastatic drugs with anti-angiogenesis therapies is important to analyze (Bergers and Hanahan 2008). This new therapeutic mandate for anti-cancer therapies can be addressed through a novel therapy comprising DEspR-inhibition, since DEspR and VEGFsp expression are detected in human endothelial cells, increased in tumor vessels, detected in cancer cells in tumor tissue arrays and in different established metastatic cancer cell lines, and since inhibition of DEspR decreases both angiogenesis and tumor cell invasiveness using corresponding established in vitro assays, as shown herein.

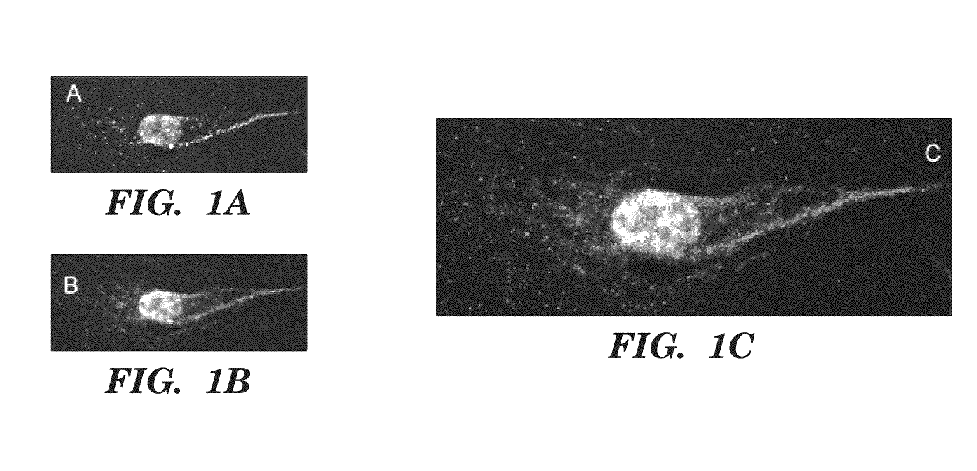

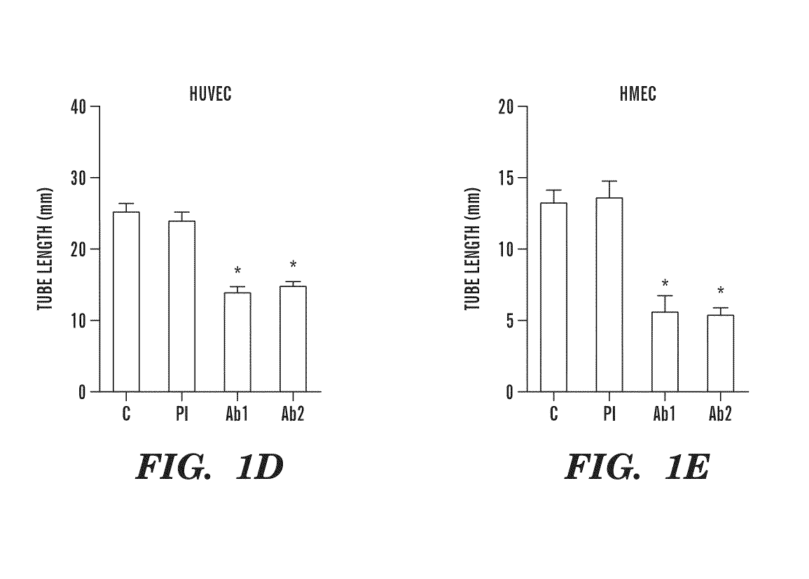

DEspR and VEGFsp were detected by immunostaining in umbilical vein endothelial cells (HUVECs) and microvascular endothelial cells (HMECS) in both basal and angiogenic tube-formation conditions (FIGS. 1A-1E). Importantly, inhibition of angiogenesis neovessel tube length was seen using both anti-DEspR (Ab1) and anti-VEGFsp (Ab2) antibodies in HUVECs (FIG. 1D) and HMECs (FIG. 1E) angiogenesis assays (Tukey's pairwise multiple comparison P <0.001 for both HUVECs and HMECs). Similar findings were observed using other angiogenesis parameters, such as neovessel branching and inter-connections made. Equally important, DEspR and VEGFsp were also detected in tumor cells, with colocalization of VEGFsp and DEspR in the cell membrane and nuclear membrane. Representative immunostaining is shown in FIGS. 1A-3C.

DEspR cell-membrane and nuclear-membrane expression were detected in multiple tumor cell types, indicating that anti-DEspR therapy is effective for different cancer types. Briefly, DEspR expression was detected in human lung non-small cell ca NCI-H727, lung giant cell tumor TIB-223/GCT; triple negative breast adenocarcinoma MDA-MB-468, bladder ca 253J BV, colon adenoca SW480, hepatocellular ca HEP3B, melanoma SK-MEL-2, osteosarcoma MG-63, ovarian adenoca HTB-161/NIH:OVCA R3, prostate adeno ca PC-3 mm2, and pancreatic ca CRL-1469/PANC-1. DEspR expression was not detected in HCI-H292 lung mucoepidermoid ca, and HEPG2 hepatocellular carcinoma, and CCL-86/Raji Burkitt's lymphoma, thus showing specificity of positive observations. Findings in NCI-727 lung ca cells were corroborated on tumor-section immunostaining of Gr.III lung adenocarcinoma.

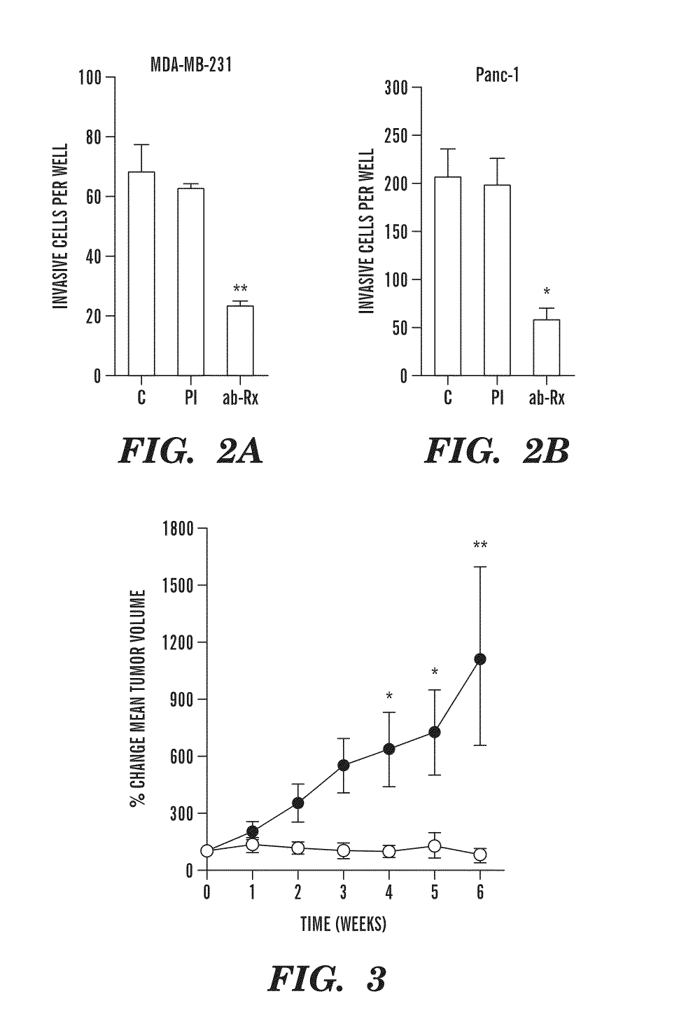

As shown in FIGS. 2A-2B, in contrast to control (C) and pre-immune ab treatment (PI), DEspR-inhibition via anti-humanDEspR antibody treatment inhibits tumor cell invasiveness in two cell lines tested, metastatic triple negative breast tumor MDA-MB-468 and pancreatic adenocarcinoma PANC-1 cell lines. The ability to target both tumor angiogenesis and tumor cell invasiveness through DEspR inhibition can more effectively address combined angiogenesis-metastasis phenotypes seen in aggressive tumors and in evasive resistance to current anti-VEGF therapies.

In vivo proof has also been demonstrated in an irradiation-induced mammary tumor model in immunocompetent rats using anti-ratDEspR antibody (Herrera et al. 2005). As shown in FIG. 3, anti-DEspR treated rats exhibited minimal tumor growth compared with mock-treated controls.

Concordantly, immunohistochemical analysis of mammary tumors showed DEspR expression in tumor cells similar to human MDA-MB-231 and MDA-MB-468 breast cancer cells, with no expression in normal breast tissue. Importantly, residual tumors in treated rats exhibited normalization of blood vessels in contrast to mock-treated tumors which showed disrupted endothelium in tumor vessels with encroachment of tumor cells into the lumen.

Clinically, the addition of VEGFsp/DEspR-targeted anti-angiogenic therapies to current VEGF/VEGFR2-targeted therapies can additively or synergistically lead to the desired endpoint of increasing overall survival in cancer patients. Given that there are several VEGF/VEGFR2 therapies already in the clinics, the translational development of anti-DEspR therapy as described herein is done in order to provide this addition.

Logistically, the experiments described herein demonstrate successful development of precursor polyclonal anti-rat DEspR antibodies (FIG. 3; Herrera et al. 2005) and polyclonal anti-human DEspR ab (FIGS. 2A-2B; Glorioso et al. 2007) that exhibit robust affinity, specificity and functionality.

There are key advantages for selecting the human monoclonal antibody therapy approaches described herein for DEspR-targeted anti-angiogenesis therapy and target-specific molecular imaging. Humanized/all human monoclonal antibody therapies (Ab-Rx) are a rapidly growing class of human therapeutics (Carter 2006) and have a relatively high success rate at 18-24% compared to new chemical entities, including small-molecule agents at 5% (Imai & Takaoka 2006).

We have developed and validated a murine monoclonal antibody specific for human-DEspR, termed herein as the 7C5B2 antibody, using a 9-amino acid (aa)-long epitope located in the extracellular amino-terminal end of hDEspR (Glorioso et al., 2007).

Briefly, mice were immunized with a KLH-conjugated antigenic peptide comprising the NH₂-terminal 9 amino acids of hDEspR, i.e., DEspR(1-9). After four injections, sera were collected for screening of antibody titer using free antigenic peptide as antigen. The mouse exhibiting the best titer was used for fusion experiments. Supernatants of fused clones were screened by ELISA using free antigenic peptide as antigen. All positive clones were transferred onto 24-well plate and re-tested/confirmed by ELISA. The 10 best clones were selected for further testing, which comprised the candidate monoclonal antibodies, anti-hDEspR monoclonal antibody. Relative affinities of prospective monoclonal antibodies were determined by ELISA using the supernatant from 10 best clones identified.

Analysis of relative monoclonal antibody affinity for antigenic hDEspR 9-aa peptide identified clones 7C5C5 and 7C5B2 as the monoclonal antibodies with strongest affinity. These two were selected for expansion and subsequent large-scale production based upon their higher affinity for the antigenic peptide.

To ascertain specificity, low- (5G12E8), mid- (2E4H6), and high-affinity (7C5B2) monoclonal antibodies were tested for western blot analysis by testing the subclone supernatant, and the subsequent purified antibody. Candidate anti-hDEspR monoclonal antibodies were specific for the predicted 10 kD protein for hDEspR. Western blot analysis was done using total cellular protein isolated from Cos1 hDEspR-transfected cells as antigen, primary antibody comprised purified antibody and subclone supernatant of 3 selected clones, 10% gel concentration in order to detect the expected 10 kD molecular weight protein of hDEspR. Nitrocellulose (PIERCE) was used with a transfer buffer of 3.07 g Tris, 14.4 g Glycine, 200 ml methanol, 800 ml dH₂O. HRP-anti mouse polyvalent immunoglobulins were used (Sigma #0412) 1:100,000; ECL reagent (SuperSignal West Femto Kit #34094), Stain reagent Kodak RP-X-Omat, and x-film (Kodak X-film #XBT-1).

The Western blot results demonstrated specificity of anti-hDEspR monoclonal antibody regardless of relative affinity, and identified more than one successful anti-hDEspR monoclonal antibody. Of the antibodies tested, the monoclonal antibody clone with highest relative affinity and specificity was clone 7C5B2.

The top candidate anti-hDEspR monoclonal antibodies were tested for inhibition of angiogenesis parameters in order to identify candidate anti-hDEspR mAb-Rxtic as anti-angiogenic using established in vitro assays.

To assess anti-angiogenic properties specific to human cells, commercially available, pre-validated established angiogenesis assays based on human umbilical vein cells (HUVECs) were used. Multiple in vitro-assay angiogenesis parameters were monitored, such as number of angiogenic tubes formed, ability of “neovessels” or tubes to branch (# branch points), ability of said neovessel branches to connect and form complex connections (# branch=connections), and robustness of angiogenesis represented by neovessel tube length (tube length in mm). Purified 7C5B2 anti-DEspR monoclonal antibody's ability to inhibit HUVECS angiogenic capacity in vitro was assessed accordingly.

An optimal effective concentration of anti-hDEspR 7C5B2 monoclonal antibody that can inhibit >80% of neovessel tube length and number of branch points was first assessed. This optimal inhibitor concentration for anti-angiogenesis efficacy was found to be 500 nM of the anti-hDEspR 7C5B2 monoclonal antibody. This concentration was then used in a series of experiments to evaluate other in vitro parameters of angiogenesis.

The anti-hDEspR 7C5B2 monoclonal antibody effectively inhibited different in vitro parameters of angiogenesis, such as number of neovessel tubes formed, branch points, branch connections and tube length. The anti-hDEspR 7C5B2 monoclonal antibody worked as well if not better than a previously validated polyclonal antibody, thus validating its potential as a monoclonal therapeutic.

The anti-hDEspR 7C5B2 monoclonal antibody was also tested for specific binding to tumor vessel endothelium and/or tumor cells in human cancer tissue arrays. The anti-hDEspR 7C5B2 monoclonal antibody was evaluated in immunohistochemical analyses of human tumor tissue-arrays comprised of core biopsy specimens representing tumors and normal tissue on the same slide. Conditions that optimized specificity and sensitivity of detection using formalin-fixed, paraffin embedded core biopsy sections were tested. Double-immunofluorescence experiments were performed in order to evaluate hDEspR expression and CD133 expression, with the latter serving as a marker for putative cancer stem cells. Antigen-retrieval was performed and used anti-hDEspR monoclonal antibody at 1:10, and commercially available anti-CD133 mAb at 1:20 dilution.

Immunohistochemical analysis of human tumor tissue-arrays using anti-hDEspR 7C5B2 monoclonal antibody detected increased expression of hDEspR in stage II-lung cancer tumor cells. Some tumor cells are double immunostain-positive for both hDEspR and CD133, with other tumor cells immunostained for CD133. These observations demonstrate that hDEspR is also present in postulated CD133-positive cancer stem cells, as well as CD133-negative tumor cells. In contrast, normal lung specimen does not exhibit any immunostaining for hDEspR or CD133. In addition, increased DEspR expression was observed in a variety of CD133+ cancer stem cell subsets, as detected by immunofluorescence with a combination of anti-DEspR, anti-CD133 and anti-CXCR4 monoclonal antibodies, including TNBC mda-mb-231 cells, pancreatic ductal adenoca Panc1 cells, glioblastoma cells, and breast cancer cells. Accordingly, in some embodiments, the compositions and methods described herein can be used in targeted treatments for tumor resistance and/or recurrence by targeting cancer stem cells or cancer initiating cells.

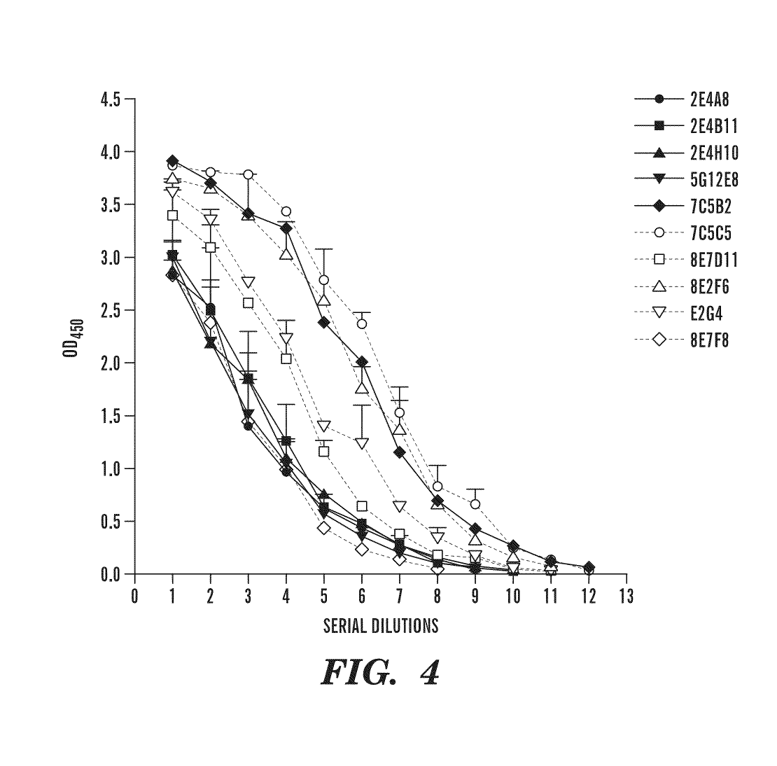

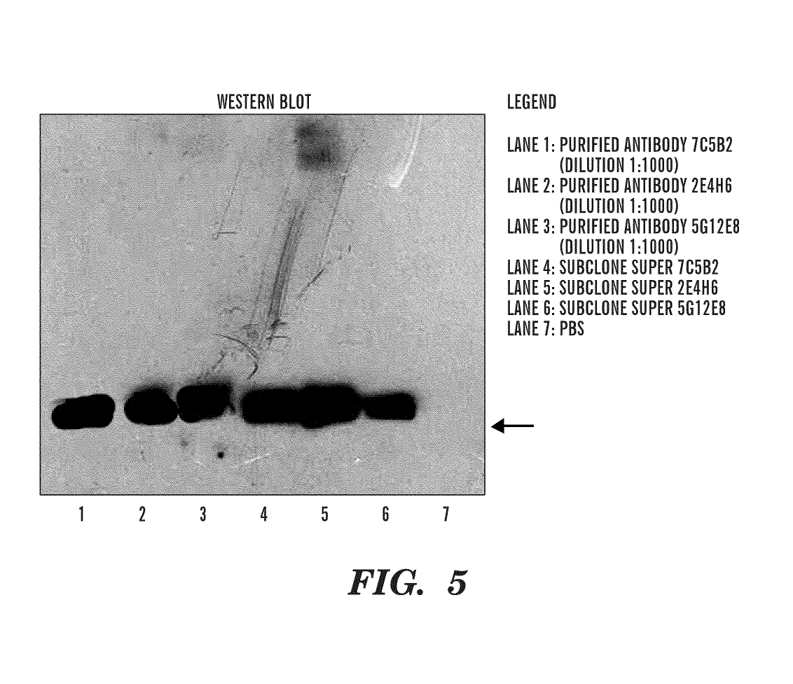

Accordingly, to summarize, this murine antibody “7C5B2” exhibited high affinity binding by ELISA to the 9 α-long epitope (FIG. 4), demonstrates specificity by western blot (FIG. 5), immunostains HUVECs undergoing tube formation (FIGS. 1A-1E), and pancreatic adenoca PANC-1, and breast cancer MDA-MB-231 cells.

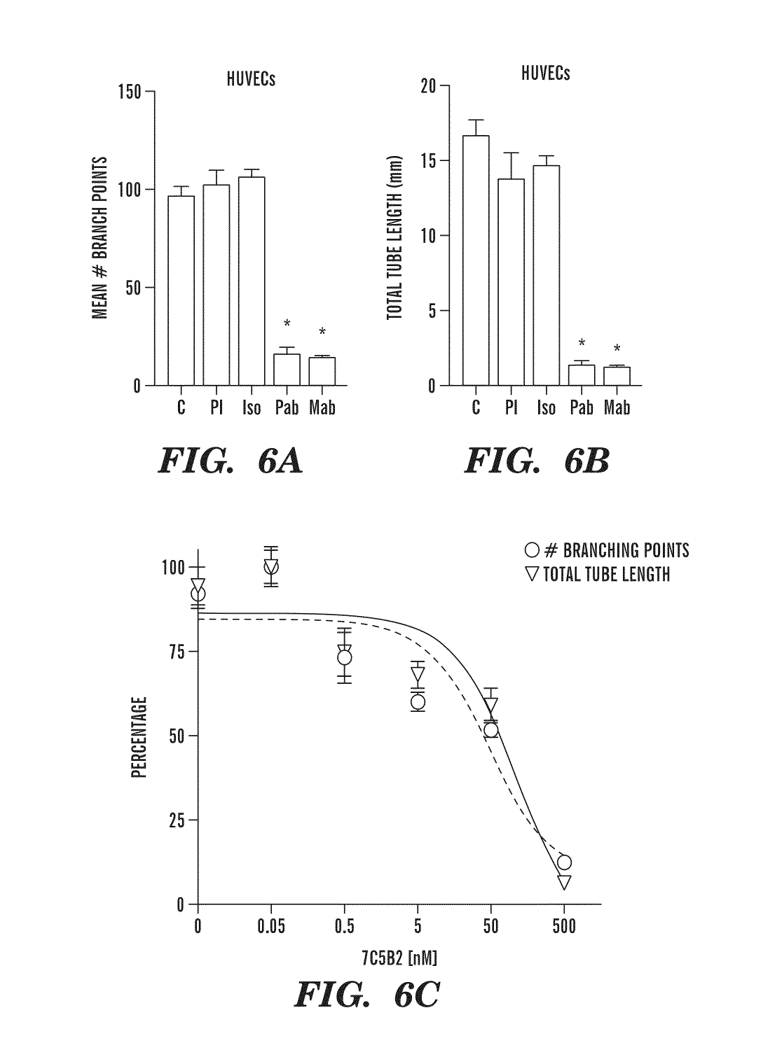

We demonstrated functional efficacy in vitro by showing that both the polyclonal (Pab) and monoclonal anti-DEspR 7C5B2, specific for human DEspR, inhibit different parameters of angiogenesis in HUVECs (FIGS. 6A-6C): mean number of branch points as a measure of neovessel complexity (FIG. 6A), and total length of tubes as a measure of neovessel density (FIG. 6B). Dose response curve for inhibition (FIG. 6C) showed equivalent robustness to inhibit both angiogenesis parameters. Importantly, murine 7C5B2 also inhibits tumor cell invasiveness in MDA-MB-468 human triple negative breast cancer and PANC-1 pancreatic cancer cell lines.

This murine anti-human DEspR monoclonal antibody 7C5B2 is thus shown to have high affinity, specificity, and functionality serves as the starting antibody for the development of anti-DEspR composite de-immunized all human antibodies, as described herein.

Accordingly, described herein are the development, characterization, and in vitro efficacy testing of anti-hDEspR composite de-immunized all human monoclonal antibody (cdHMAb) for use as novel antibody therapies aimed at addressing evasive and intrinsic resistances to current anti-VEGF/VEGFR2 antiangiogenic therapies.

We have selected Antitope's Composite Human Antibody technology to generate anti-hDEspR deimmunized human monoclonal antibodies for antibody therapeutics (Antitope, 2010). This technology generates de-immunized 100% human antibodies at the outset, in contrast to non-deimmunized human antibodies derived from phage and transgenic mice technologies. Briefly, composite human antibodies comprise multiple sequence segments (‘composites”) derived from V-regions of unrelated human antibodies are selected to maintain monoclonal antibody sequences critical for antigen binding of the starting murine precursor anti-human DEspR monoclonal antibody, and are filtered for the presence of potential T-cell epitopes using proprietary “in silico tools” (Holgate & Baker 2009). The close fit of human sequence segments with all sections of the starting antibody V regions and the elimination of CD4+ T cell epitopes from the outset circumvent immunogenicity in the development of ‘100% human’ therapeutic antibodies while maintaining optimal affinity and specificity through the prior analysis of sequences necessary for antigen-specificity (Holgate & Baker 2009). Immunogenicity can hinder clinical applications of 100% human monoclonal antibodies (Chester et al. 2009).

Briefly, “composite human antibodies” comprise multiple sequence segments (“composites”) derived from V-regions of unrelated human antibodies that are selected to maintain monoclonal antibody sequences critical for antigen binding of the starting murine precursor anti-human DEspR monoclonal antibody, such as 7C5B2 antibody, and which have all been filtered for the presence of potential T-cell epitopes using “in silico tools” (Holgate & Baker, 2009). The close fit of human sequence segments with all sections of the starting antibody V regions and the elimination of CD4+ T cell epitopes from the outset allow this technology to circumvent immunogenicity in the development of ‘100% human’ therapeutic antibodies while maintaining optimal affinity and specificity through the prior analysis of sequences necessary for antigen-specificity (Holgate & Baker 2009).

As described herein, structural models of mouse anti-hDEspR antibody V regions were produced using Swiss PDB and analysed in order to identify important “constraining” amino acids in the V regions that were likely to be essential for the binding properties of the antibody. Residues contained within the CDRs (using Kabat definition) together with a number of framework residues were considered to be important. Both the V_{H} and V_{L} (V_{κ}) sequences of anti-hDEspR, as described herein as SEQ ID NO: 4 and SEQ ID NO: 9, comprise typical framework residues and the CDR1, CDR2, and CDR3 motifs are comparable to many murine antibodies.

From the above analysis, it was determined that composite human sequences of anti-hDEspR can be created with a wide latitude of alternatives outside of CDRs, but with only a narrow menu of possible alternative residues within the CDR sequences. Analysis indicated that corresponding sequence segments from several human antibodies could be combined to create CDRs similar or identical to those in the murine sequences. For regions outside of and flanking the CDRs, a wide selection of human sequence segments were identified as possible components of novel anti-DEspR composite human antibody V regions for use with the compositions and methods described herein (see, for example, Table 1).

Based upon these analyses, a large preliminary set of sequence segments that could be used to create novel anti-DEspR composite human antibody variants were selected and analysed using ITOPE™ technology for in silico analysis of peptide binding to human MHC class II alleles (Perry et al 2008), and using the TCED™ (T Cell Epitope Database) of known antibody sequence-related T cell epitopes (Bryson et al 2010). Sequence segments that were identified as significant non-human germline binders to human MHC class II or that scored significant hits against the TCED™ were discarded. This resulted in a reduced set of segments, and combinations of these were again analysed, as above, to ensure that the junctions between segments did not contain potential T cell epitopes. Selected segments were then combined to produce heavy and light chain V region sequences for synthesis. Exemplary heavy chain V region sequences provided herein and generated using the above-described methods include SEQ ID NO: 13-SEQ ID NO: 17. Exemplary heavy chain V region sequences provided herein and generated using the above-described methods include SEQ ID NO: 18-SEQ ID NO: 19.

In vitro efficacy of the antibodies described herein are assessed by examining dose response-inhibition of angiogenesis of HUVECs (human umbilical vein cells) and HMECs (adult human microvascular endothelial cells) in angiogenesis assays (see FIGS. 1A-1E, 6A-6C), which in some embodiments are set-up with co-cultured cancer cells, such as PANC-1 and MDA-MB-468, and in some embodiments in normoxia and hypoxia (2% O2) conditions. Both HUVECs and HMECs are used for the following reasons: HUVECs is the standard in the field, but as these cells are umbilical vein derived, and adult microvascular endothelial cells (HMECs) are also used. In addition, angiogenesis is assessed with co-cultured cancer cells, in addition to the fetal bovine serum that is usually added in angiogenesis assays, in order to better simulate angiogenic factors that cancer cells produce which contribute to evasive and intrinsic resistance.

In some embodiments, since hypoxia is one of the triggers for angiogenesis, and one of the contributing factors suspected of underlying evasive resistance to current anti-VEGF therapies, in vitro efficacy assays are conducted in normoxia and in 2% O2 hypoxia. Composite deimmunized monoclonal antibody-mediated inhibition of tumor cell invasiveness in vitro is analyzed using MDA-MB-468 and PANC-1 cells and by using established quantitative assays. These are also done in normoxia and 2% O2-hypoxia conditions, to test a more aggressive tumor cell phenotype known to be associated with hypoxia.

The effects of anti-hDEspR inhibition are compared to controls, which can include untreated controls, isotype controls, murine precursor anti-hDEspR monoclonal antibody controls, and bevacizumab controls. Each point for angiogenesis and tumor cell invasiveness assays are done using at least 5 replicates. Furthermore, for the top 2 candidate-leads, dose response curve inhibition responses are also performed, where each dosage is studied using at least 5 replicates.

Assays can be analyzed by one way ANOVA and multiple pairwise comparison to assess significant changes. Mean levels of %-inhibition from control by each candidate lead (e.g., 5-10) are used to rank them according to different assays, and the highest ranked two identifies the top-2 leads corresponding to best inhibitor of angiogenesis and tumor cell invasiveness in both for example, normoxia and hypoxia conditions, and in both, for example, MDA-MB-468 and PANC-1 cancer cell lines respectively.

Tumor array analysis is done to corroborate specificity and sensitivity of each to detect tumor cells and tumor neovessels in tissue arrays of human biopsy core samples form different cancer tissue types. This is performed on a tissue array panel representing solid tumors from brain, pancreas, lung, breast, ovarian, prostate, bladder, colon, stomach. Results are analyzed for specificity given the same immunochemistry conditions used in validation of the murine precursor anti-hDEspR Mab-H1. As shown, there is minimal DEspR expression in normal human pancreas, whereas in stage 1V pancreatic cancer exhibits increased DEspR expression in pancreatic tumor cells and tumor blood vessels. The composite deimmunized monoclonal antibody candidate leads are ranked and the top-2 that have the best detection of tumor cells and tumor neovessels with optimal signal to noise ratio in tumor tissue array immunohistochemistry are determined. This can be compared to tumor-array immunostaining observations obtained with the murine precursor anti-hDEspR Mab.

In addition to de-immunizing the antibodies described herein using in silico screening of T-cell epitopes to minimize and reduce immunogenicity, the composite anti-hDEspR composite deimmunized monoclonal antibodies are tested in vitro for immunogenicity in order to select for the least immunogenic composite all human Mab. Immunogenicity screening can be performed using a representative of 50 donors, which has proven to correlate with clinical observations (Baker & Jones 2007).

Immunogenicity testing, along with the other in vitro assays of specificity and efficacy allows for the selection of a top anti-hDEspR lead, based on a combination of factors, including best affinity (ELISA), specificity (western blot analysis), in vitro efficacy (inhibition of angiogenesis and tumor cell invasiveness) and lowest immunogenicity. A priori ascertainment of low immunogenicity by elimination of T cell epitopes in the composite antibody process, and low immunogenicity ascertainment by using ex vivo T cell assay technology are important translational research steps, since high immunogenicity limits ab therapeutic efficacy (Iwai & Takaoka 2006) despite target-specificity and total humanization as has been discussed in clinical studies for Infliximab, Alemtuzumab (review by Baker & Jones 2007).

The top composite deimmunized monoclonal antibodies leads are tested for in vivo efficacy by testing anti-DEspR-mediated inhibition of tumor growth, angiogenesis and metastasis in established human cancer cell line xenograft and metastasis models in immuno-compromised mice. Cancer tissue types representative of evasive resistance (breast cancer) and intrinsic resistance (pancreatic cancer) as observed in published reports are also tested. For example, MDA-MB-231 breast cancer and PANC-1 pancreatic carcinoma cell lines are used, since both can be used to generate xenograft and metastasis spleen-infusion models. For MDA-MB-231 orthotopic and metastasis models nude mice are used (Oh et al. 2009, Roland et al. 2009). For PANC-1 xenograft subcutaneous models nude mice are used as described (Zheng et al. 2008) and NOG mice for PANC-1 metastasis model as described (Suemizu et al. 2007).

Through the strategic use of anti-humanDEspR-specific (e.g., composite deimmunized monoclonal antibody primary lead) and anti-human-VEGF-specific (bevacizumab) antibodies, and a murine-DEspR-specific Mab, 1) efficacy of anti-DEspR therapy compared with anti-VEGF therapy alone can be assessed, and 2) determination of synergistic efficacy using a combination of anti-DEspR and anti-VEGF antibodies.

Treatment in xenograft models begin when tumors are 200-300 mm in size to simulate clinical cancer therapy scenarios. To assess anti-DEspR therapy efficacy in metastasis models, a sustained treatment regimen begun 5 days after the intrasplenic infusion of cancer cells is assessed, as described (Oh et al. 2009). To assess whether anti-DEspR therapy induces increased risk for metastasis observed with sunitinib (Ebos et al. 2009), Ebos's experiment are performed, whereby anti-murineDEspR Mab is infused daily for 7 doses beginning 7 days prior to cancer cell infusion. 250 ug is used for each antibody-therapeutic given IP 2×/week as described for bevacizumab (Roland et al. 2009), and 3× per week for anti-DEspR (Herrera et al. 2005).

Treatment outcomes are assessed by multifaceted parameters: serial imaging of tumor volume and tumor angiogenesis for orthotopic mammary and subcutaneous pancreatic tumors by, for example, high-resolution Vevo770 ultrasound imaging and power Doppler analysis. Overall survival is determined, and at this endpoint, repeat ultrasound imaging and histological analysis of tumor size and angiogenesis is done, along with histological analysis of malignancy phenotype: nuclear grade, tumor cell invasion of stroma, tumor cell vascular mimicry, loss of integrity of tumor neovessels and macrophage infiltrates.

Heterozygous DEspR+/− mice live beyond 1 year and breed, which is in contrast to VEGF+/− haplodeficiency which is embryonic lethal at E11.5. However, since adverse effects have been observed in patients on anti-VEGF (bevacizumab) and anti-VEGFR2 (sunitinib, sorafanib) therapies, the anti-humanDEspR-specific antibodies described herein are also tested for these effects. Analysis of parameters of potential adverse effects are done in PANC-1 and MDA-MB-231 xenograft models treated with cdHMAb-H1 and mDEspR-Mab. For example, potential a) cardiotoxicity can be monitored by serial non-invasive ultrasound cardiac function analysis; b) hypertension can be monitored by tail cuff BP; c) bowel perforation can be monitored on post-mortem anatomical inspection at endpoint; d) bleeding, thrombosis can be monitored by examination and vascular ultrasound and Doppler flow analysis, and e) toxicity screen can be performed, such as liver function tests, renal function tests, complete blood count, blood chemistries at endpoint of study. These parameters are compared in mock-treated age-matched tumor model controls.

Analysis of Molecular Imaging of Tumor Angiogenesis and Tumor Cell Vascular Mimicry Changes in Response to Therapy by Contrast-Enhanced Ultrasound Imaging of DEspR-Targeted Neovessels Compared with VEGFR2-Targeted Tumor Neovessels.

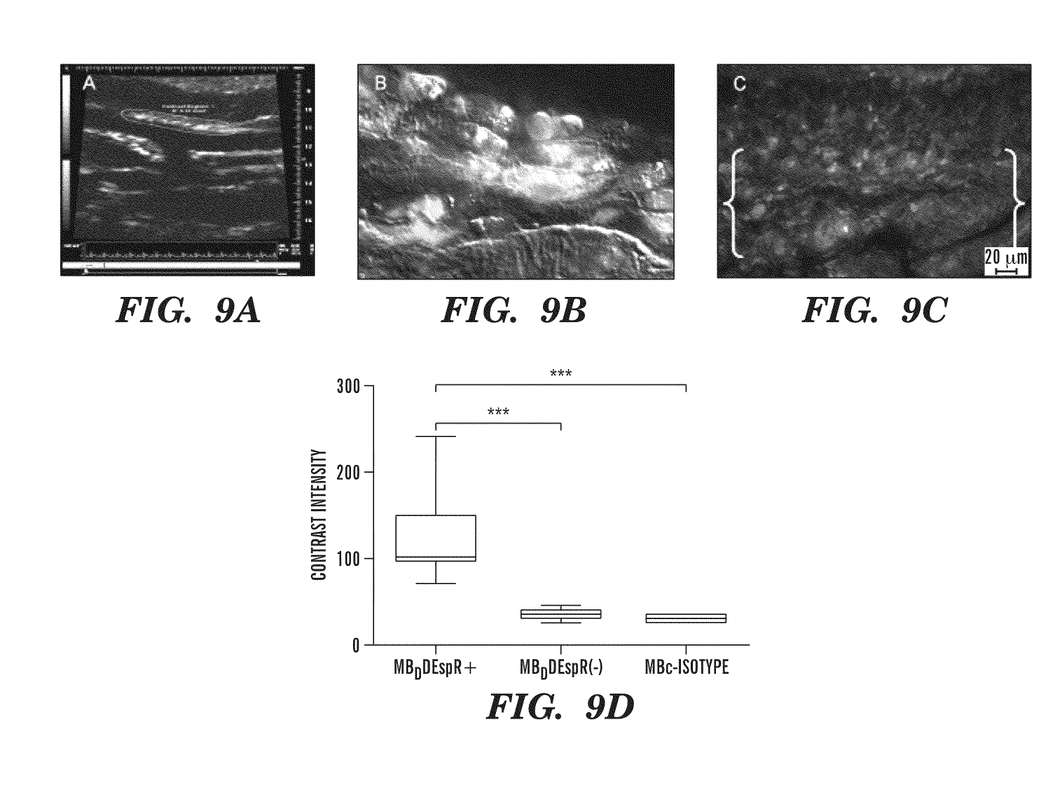

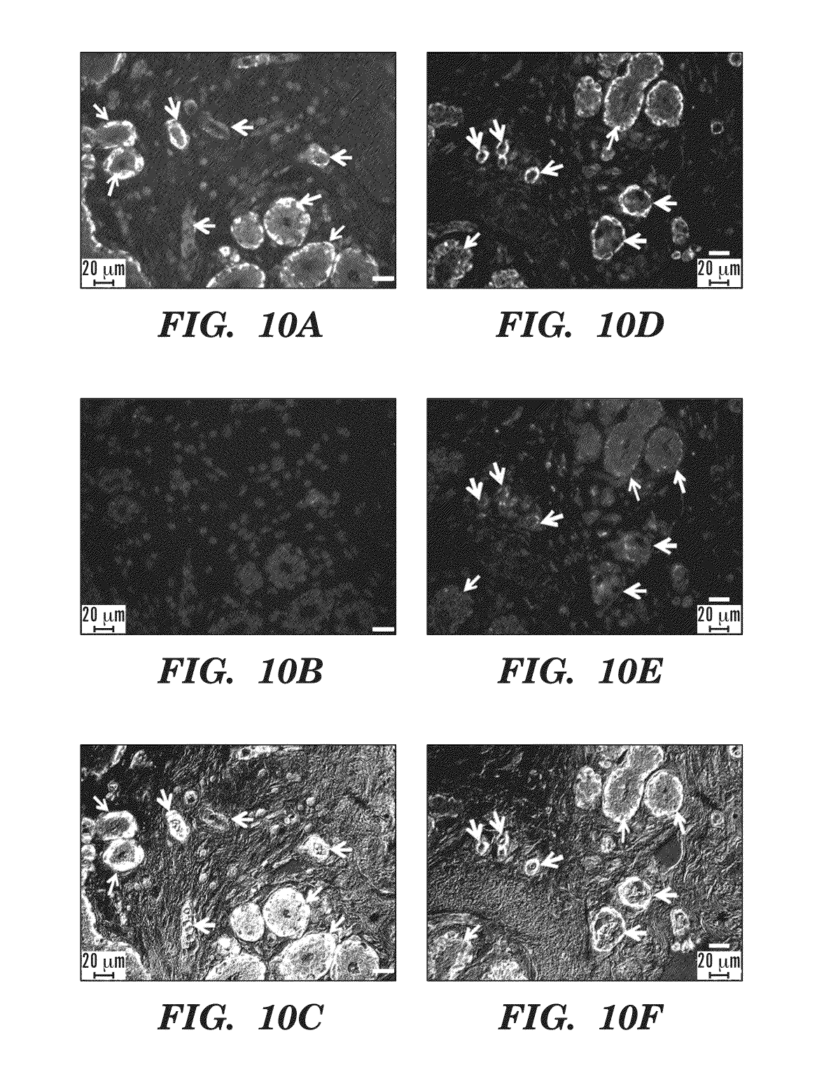

Molecular imaging of angiogenesis in tumors has been demonstrated by contrast-enhanced ultrasound imaging using anti-VEGFR2 antibody-directed microbubbles with imaging and contrastenhanced analysis done using the VisualSonics Vevo770 high-resolution ultrasound system (Willmann et al. 2007). We have used this same system to detect anti-DEspR antibody-directed microbubbles in carotid artery disease vasa vasorum angiogenesis in a transgenic rat atherosclerotic model associated with carotid artery disease progression and stroke risk (Decano et al. 2010). As shown in FIGS. 9A-9D, DEspR-targeted molecular imaging (9A) detects DEspR+endothelial lesions (9B) and vasa vasorum angiogenesis (9C). Quantitation of contrast intensity is done using integrated software (9D).

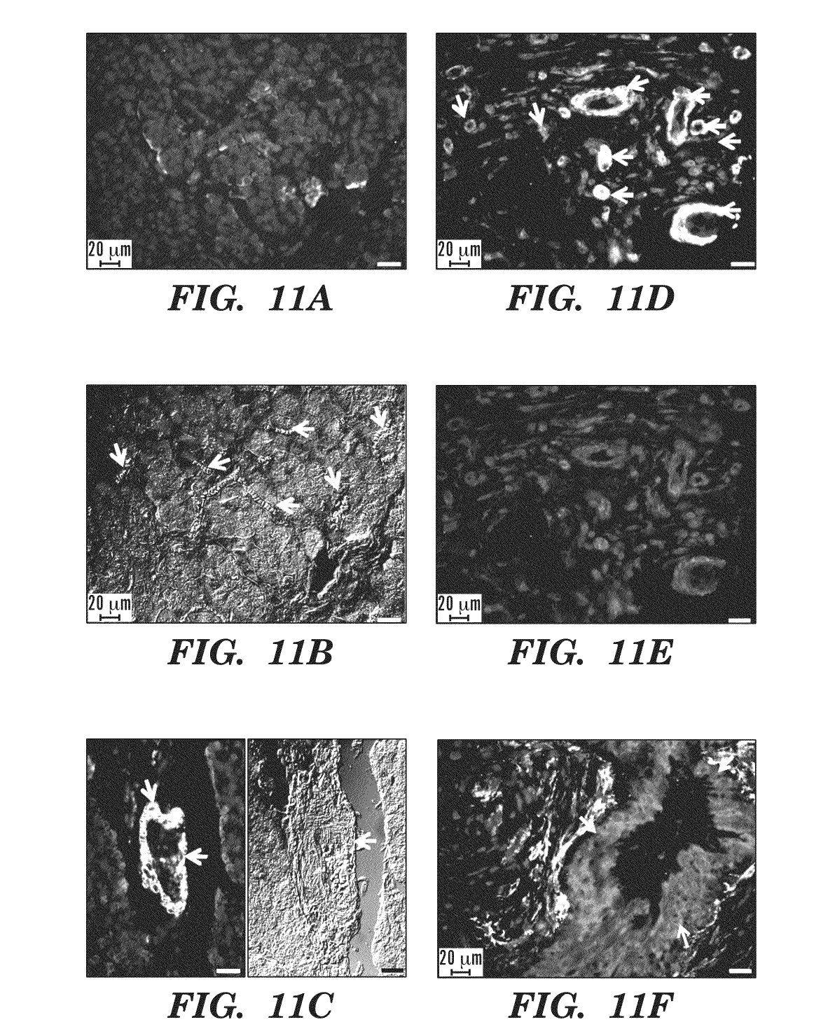

Immunohistochemical analysis of DEspR expression in human breast tissue was also performed using an anti-DEspR monoclonal antibody (FIGS. 10A-10C) normal; Grade-1, T1 invasive ductal carcinoma (FIGS. 10D-10F). FIG. 10A shows normal breast tissue: 3×-overlay of DEspR, aSMA and DAPI nuclear stain detects aSMA expression in mammary myoepithelial cells but no expression of DEspR in epithelial cells and microvessels. FIG. 10B shows 2×-immunofluorescence overlay of DEspR and DAPI nuclear stain and confirms absence of DEspR expression in normal breast tissue. FIG. 10C shows a 4×-overlay of DEspR, aSMA, DAPI immunofluorescence and diffusion contrast imaging (DIC) that delineates tissue morphology, expression of aSMA. and non/minimal-expression of DEspR in normal mammary epithelium and endothelium. FIG. 10D is a 3×-Overlay of DAPI, aSMA and DEspR immunofluorescence in Gr.I-T1 invasive ductal carcinoma that detects DEspR expression in vascular endothelium, and co-localization with aSMA in mammary tissue. FIG. 10E is a 2×-overlay of DAPI and DEspR of breast cancer shown in panel 10D that highlights DEspR expression. FIG. 10F is a 4×-overlay of DAPI, aSMA, DEspR, DIC to elucidate DEspR spatial expression with tissue morphology of epithelial cells and microvessels. bar=20 microns.

DEspR-targeted molecular imaging is used to test composite deimmunized monoclonal antibodies as the targeting module for molecular imaging applicable to xenograft tumor cell vascular mimicry, and microbubbles are confined to the vascular lumen. MouseDEspR-specific molecular imaging using composite deimmunized monoclonal antibodies as described herein is performed in order to monitor mouse-derived tumor angiogenesis, and is compared to VEGFR2-specific molecular imaging. The observations described herein provide proof that composite deimmunized monoclonal antibodies specific for DEspR can serve as the targeting module for molecular imaging of tumor cell vascular mimicry in a mouse model; that molecular imaging of DEspR expression provides a translatable diagnostic in vivo imaging modality to assess tumor angiogenesis, and that comparative analysis of DEspR-specific molecular imaging provides new insight into the differential contribution of tumor cell vascular mimicry and tumor angiogenesis.

Both TNBC xenograft orthotopic and PANC-1 xenograft heterotopic tumor models, as well as a PANC-1 intrasplenic-infusion liver metastasis model are used for molecular imaging experiments. Isotype-antibody molecular imaging is used as a control to demonstrate specificity of DEspR-positive molecular imaging. Identical conditions are followed for anti-DEspR and anti-VEGFR2 molecular imaging in order to validate comparative analysis. For example, a composite deimmunized monoclonal antibody can be used to target tumor cell vascular mimicry; an anti-DEspR composite deimmunized monoclonal antibody can be used to target mouse neovessel formation monoclonal antibody in human xenograft tumors; anti-VEGFR2 can be used as a comparative benchmark, and an isotype antibody can be used as a negative control.

### REFERENCES

Baker M P, Jones T D. 2007. Identification and removal of immunogenicity in therapeutic proteins. Curr Opin Drug Disc Dev 10:219-227.
Bergers G, Hanahan D. 2008. Modes of resistance to anti-angiogenic therapy. Nature Reviews—Cancer 8:592-603.
Bocci G, Man S, Green S K, Francia G, Ebos J M, du Manoir J M, Weinerman A, Emmenegger U, Ma L, Thorpe P, Davidoff A, Huber J, Hicklin D J, Kerbel R S., 2004. Increased plasma VEGF as a surrogate marker for optimal therapeutic dosing of VEGF receptor-2 monoclonal antibodies. Cancer Res 64:6616-6625.
Butler J M, Kobayashi H, Rafii S. 2010. Instructive role of the vascular niche in promoting tumor growth and tissue repair by angiogenic factors. Nature Reviews—Cancer 10:138-146.
Carmeliet P. 2005. Angiogenesis in life, disease and medicine. Nature 438:932-936.
Carter P J. 2006. Potent antibody therapeutics by design. Nature Reviews Immunology 6:343-357.
Chester K A, Baker M, Mayer A. 2005. Overcoming the immunologic response to foreign enzymes in cancer therapy. Expert Rev Clin Immunol 1:549-559.
Crawford Y, Ferrara N. 2008. Mouse models to investigate anti-cancer effects of VEGF inhibitors. Methods Enzymol 445:125-139.
Decano J L, Matsubara Y, Moran A M, Ruiz-Opazo N, Herrera V L M. 2010. Dual endothelin-1/VEGFsp receptor (DEspR) roles in adult angiogenesis in despr+/− knockout mice and carotid artery disease rat model. (Manuscript submitted to Circulation.)
Ebos J M, Lee C R, Cruz-Munoz W, Bjarnason G A, Christensen J G, Kerbel R S. 2009. Accelerated metastasis after short-term treatment with a potent inhibitor of tumor angiogenesis. Cancer Cell 15:232-239.
Ferrara N. 2009. Pathways mediating VEGF-independent tumor angiogenesis. Cytokine Growth Factor doi:10.1016/j.cytogfr.2009.11.003.
Glorioso N, Herrera V L M, Bagamasbad P, Filigheddu F, Troffa C, Argiolas G, Bulla E, Decano J L, Ruiz-Opazo N. 2007. Association of ATP1A1 and Dear SNP-haplotypes with essential hypertension: sex-specific and haplotype-specific effects. Circ Res 100: 1522-1529.
Herrera V L M, Ponce L R, Bagamasbad P D, VanPelt B D, Didishvili T, Ruiz-Opazo N. 2005. Embryonic lethality in Dear gene-deficient mice: new player in angiogenesis. Physiol Genomics 2005; 23:257-268.
Holgate R G E, Baker M P. 2009. Circumventing immunogenicity in the development of therapeutic antibodies. IDrugs 12:233-237.
Imai K, Takaoka A. 2006. Comparing antibody and small-molecule therapies for cancer. Nature Reviews Cancer 6:714-727.
Jubb A M, Oates A J, Holden S, Koeppen H.2006. Predicting benefit from anti-angiogenic agents in malignancy. Nature Reviews—Cancer 6:626-635.
Loges S, Schmidt T, Carmeliet P. 2010. Mechanisms of resistance to anti-angiogenic therapy and development of third-generation anti-angiogenic drug candidates. Genes & Cancer 1:12-25.
Oh S, Stish B, Sachdev D, Cehn H, Dudek A, Vallera D A. 2009. A novel reduced immunogenicity bispecific targeted toxin simultaneously recognizing human epidermal growth factor and interleukin-4 receptors in a mouse model of metastatic breast carcinoma. Clin Cancer Res 15:6137-6147 (PMCID: PMC2756320 [Available on 2010/10/1]).
Paez-Ribes M, Allen E, Hudock J, Takeda T, Okuyama H, Vinals F, Inoue M, Bergers G, Hanahan D, Casanovas O. 2009. Antiangiogenic therapy elicits malignant progression of tumors to increased local invasion and distant metastasis. Cancer Cell 15:220-231.
Roland C L, Dineen S P, Lynn K D, Sullivan L A, Dellinger M T, Sadegh L, Sullivan J P, Shames D S, Brekken R A. 2009. Inhibition of vascular endothelial growth factor reduces angiogenesis and modulates immune cell infiltration of orthotopic breast cancer xenografts. Mol Cancer Ther 8:1761-1771.
Stewart D J, Kutryk M J, Fitchett D, Freeman M, Camack N, Su Y, Della Siega A, Bilodeau L, Burton J R, Proulx G, Radhakrishnan S; NORTHERN Trial Investigators. 2009. VEGF gene therapy fails to improve perfusion of ischemic myocardium in patients with advanced coronary disease: results of the NORTHERN trial. Mol. Ther. 17:1109-1115.
Suemizu H, Monnai M, Ohnishi Y, Ito M, Tamaoki N, Nakamura M. 2007. Identification of a key molecular regulator of liver metastasis in human pancreatic carcinoma using a novel quantitative model of metastasis in NOD/SCID/γc null (NOG) mice. Int J Oncology 31:741-751.
Willett C G, Boucher Y, Duda D G, diTomaso E, Munn L L, Tong R T, Kozin S V, Petit L, Jain R K, Chung D C, Sahani D V, Kalva S P, Cohen K S, Scadden D T, Fischman A J, Clark J W, Ryan D P, Zhu A X, Blaszkowsky L S, Shellito P C, Mino-Kenudson M, Lauwers G Y. 2005. Surrogate markers for antiangiogenic therapy and doselimiting toxicities for Bevacizumab with radiation and chemotherapy: continued experience of a phase I trial in rectal cancer patients. J Clin Oncol. 23:8136-8139.
Willmann J K, Paulmurugan R, Chen K, Gheysens O, Rodriguez-Porcel M, Lutz A M, Chen I Y, Chen X, Gambhir S S. 2008. US imaging of tumor angiogenesis with microbubbles targeted to vascular endothelial growth factor receptor type 2 in mice. Radiology 246:508-518.
Zheng X, Cui X X, Huang M T, Liu Y, Shih W J, Lin Y, Lu Y P, Wagner G C, Conney A H. 2008. Inhibitory effect of voluntary running wheel exercise on the growth of human pancreatic PACN-1 and prostate PC-3 xenograft tumors in immuno-deficient mice. Oncology Rep 19:1583-1588 (PMCID: PMC2825748).

### Example 2

### Molecular Imaging of Vasa Vasorum Neovascularization Via Despr-Targeted Contrast-Enhanced Ultrasound Micro-Imaging in Transgenic Atherosclerosis Rat Model

Given that carotid vasa vasorum neovascularization is associated with increased risk for stroke and cardiac events, the in vivo study described herein was designed to investigate molecular imaging of carotid artery vasa vasorum neovascularization via target-specific contrast-enhanced ultrasound (CEU) micro-imaging. Accordingly, molecular imaging was performed in male transgenic rats with carotid artery disease (CAD) and non-transgenic controls using DEspR (dual endothelin1/VEGFsp receptor)-targeted microbubbles (MB_{D}) and the Vevo770 micro-imaging system and CEU-imaging software.

It was found that DEspR-targeted CEU-positive imaging exhibited significantly higher contrast intensity signal (CIS)-levels and pre-/post-destruction CIS-differences in 7/13 transgenic rats, in contrast to significantly lower CIS-levels and differences in control isotype-targeted microbubble (MB_{C})-CEU imaging (n=8) and in MB_{D} CEU-imaging of 5/5 non-transgenic control rats (P <0.0001). Ex vivo immunofluorescence analysis demonstrated binding of MB_{D} to DEspR-positive endothelial cells, and association of DEspR-targeted increased contrast intensity signals with DEspR expression in vasa vasorum neovessel and intimal lesions. In vitro analysis demonstrated dose-dependent binding of MB_{D} to DEspR-positive human endothelial cells with increasing % cells bound and number of MB_{D} per cell, in contrast to MB_{C} or non-labeled microbubbles (P <0.0001).

The dual endothelin-1 (ET1)/vascular endothelial growth factor-signal peptide (VEGFsp) receptor or DEspR (formerly dear gene as deposited in GenBank) [1] plays a key role in developmental angiogenesis deduced from the embryonic lethal phenotype exhibited by despr^{-/-} knockout mice due to absent embryonic and extraembryonic angiogenesis, aborted dorsal aorta vasculogenesis, and abnormal cardiac development [2]. While exhibiting similar abnormal vasculogenesis and angiogenesis phenotypes with VEGF^{+/-} haploinsufficient mice, despr^{-/-} null mice exhibit distinct neural tube phenotypes [2-4]. Consistent with its role in developmental angiogenesis, DEspR inhibition results in decreased tumor angiogenesis and tumor growth in adult rat mammary tumors and mouse melanomas [2].

Development of target-specific contrast enhanced ultrasonography (CEU)-imaging, herein referred to as “molecular imaging” of vascular disease neovascularization is important since carotid artery vasa vasorum neovascularization is associated with increased risk for stroke [5, 6]. However, successful molecular imaging of vasa vasorum neovessels has not been reported, although detection by non-targeted CEU-imaging has [7]. On the other hand, successful molecular imaging in different disease models detecting different targets[8,9] has shown the potential of molecular imaging in different disease contexts, such as αvβ3 in tumor and hind limb ischemia angiogenesis [10,11], VEGFR2 in tumor angiogenesis [12], ICAM-1 in transplant rejection [13], L-selectin in malignant lymphnodes [14], and ICAM-1 and VCAM-1 in atherosclerosis [15], P-selectin in myocardial ischemia [16,17], GIIb/IIIa and fibrinogen in thrombosis [18,19]. Molecular imaging of vascular disease neovascularization in studies targeting VEGFR2-, ICAM-1 and VCAM-1 did not detect vasa vasorum neovessels in a hyperlipidemic rabbit model of injury-induced vascular neovascularization [9, 20].

Demonstrated herein is molecular imaging of DEspR in carotid artery lesions and expanded vasa vasorum neovessels in transgenic-hyperlipidemic, hypertensive carotid artery disease rat model.

### Materials and Methods

Animals. In order to facilitate molecular imaging studies of pathological angiogenesis in vascular lesions or in expanded vasa vasorum neovessels, a carotid artery disease rat model with hypertension-atherosclerosis as risk factors, the Tg25[hCETP] Dahl-S rat model, Tg25, transgenic for human cholesteryl ester transfer protein which develops accelerated stroke [21] or later-onset coronary heart disease, was selected [22]. 4-month old transgenic male rats (n=13) projected to be around early-midpoint along the disease course of stroke [21] or coronary atherosclerosis phenotype [22], were studied for DEspR-targeted molecular imaging (n=13). MB_{D}-infused non-transgenic, non-atherosclerotic littermates were studied as negative biological controls (n=5). Isotype-specific MB_{S}-infused transgenic rats (n=8), with the following subgroups: 4 transgenic rats which exhibited MB_{D}-specific CEU-positive imaging, and 4 de novo transgenic rats, were studied concurrently as negative imaging controls.

Target-specific CEU-molecular imaging. The VEVO®770 high resolution ultrasound system with contrast mode software, and streptavidin-coated “target ready” MicroMarker microbubbles (VisualSonics Inc, Canada) previously validated for molecular imaging of VEGFR2 on tumor angiogenesis in mice was used [12]. To target the microbubble to rat DEspR-positive endothelial cells, target ready-MicroMarker microbubbles were linked to biotinylated anti-DEspR antibody (MB_{D}) via streptavidin-biotin coupling. For control, target ready-MicroMarker microbubbles were linked to biotinylated, isotype-antibody (MB_{C}). Each bolus comprised of 3−4×10⁸ microbubbles in 200-microliters saline, infused into the rat tail vein over 8-seconds.

CEU-imaging of rat carotid arteries comprised a sequence of steps aimed at optimizing MB-target binding, eliminating confounders, and ascertaining reproducible CEU-imaging. Baseline images of the carotid artery were first obtained and immobilized the scanhead to maintain the optimal B-mode view of the common, external, and internal carotid arteries in one 2D image. One minute after MB bolus infusion, the MB blood pool was documented by B-mode imaging for all rats to ascertain MB infusion and to demonstrate absence of contrast intensity in surrounding tissue. A wait of 4-5 minutes was taken to allow MB_{D} adherence to DEspR-positive endothelial targets [12], and to allow clearance of unbound circulating microbubbles [23]. Clearance of most circulating MBs facilitates detection of increased contrast intensity signals due to adherent MB s validated for detection using the VEVO®770 imaging system [23]. Adherent MBs were defined by the loss of contrast-intensity upon acoustic destruction performed using pre-set Contrast Enhanced software (VisualSonics, Inc, Canada) as described [12].

Four regions of interest (ROI) on the carotid artery were monitores: the common carotid artery, bifurcation, external and internal carotid arteries. Quantitation of contrast intensity signals (CIS) resulting from backscatter of adherent targeted-microbubbles was done using contrast-enhanced analysis program validated for the VEVO®770 imaging platform (VisualSonics Inc, Canada) detecting pre- and post-acoustic disruption contrast intensity signals. The contra-lateral carotid artery was checked immediately, and the same CEU-imaging protocol followed. After a 20-minute interval to allow complete clearance of any residual MBs, a pre-set destruction sequence was performed for subsequent CEU-imaging with isotype-specific MB_{C}s following identical procedures. For quantitative comparative analyses, the difference in contrast intensity signals between pre- and post-acoustic destruction, CIS-difference, as well as their respective pre-destruction CIS-peak levels were studied for each carotid artery per rat.

Histology and Immunofluorescence Staining of Rat Carotid Arteries. After CEU-imaging, carotid arteries were collected en bloc preserving the surrounding tissue around the common (CCA), external (ECA) and internal (ICA) carotid arteries including the carotid artery bifurcation. The ECA was cut longer than the ICA to be able to distinguish the two. Longitudinal serial sections were obtained per carotid artery (50-100 sections) and staining every 10th slide with Masson-trichrome allowed proper orientation and site-specific analyses corresponding to ROIs in CEU-imaging. The flanking serial sections to MT-stained slides of interest were then immunostained. Double immunofluorescence staining was done on deparaffinized sections via sequential antigen retrieval, treatment to reduce background, blocking, incubation with primary antibody at 4° C. overnight, secondary antibody incubation overnight at 4° C. with AlexaFluor 568 goat anti-mouse IgG and ALEXAFLUOR® 488 goat anti-rabbit IgG, washing, and mounting using PROLONG®Gold with DAPI (Invitrogen, CA). Negative controls were run using rabbit-isotype antibody for anti-rat DEspR antibody. A Zeiss Axioskop2plus microscope was used for fluorescence imaging and differential interference contrast (DIC) photomicroscopy to provide morphological information overlay to immunostained sections. Low 2.5× magnification was used for proper orientation and site-specific identification along the carotid artery.

In vitro analysis of MB_{D} and DEspR positiveendothelial cell interactions Human-specific DEspR-targeted MB_{D}S were made following identical procedures for rat-specific DEspR molecular imaging with the exception of the use of an anti-humanDEspR monoclonal antibody. Fixed numbers of human umbilical vein endothelial cells (HUVECs) were seeded onto IBIDI perfusion 6-lane μ-slide VI (ibidiGmbH, Germany). After 24 hours, MB_{D}-type microbubbles were infused at the following MB-cell ratios: 8×, 80×, and 800×. Negative controls comprised of 800×MB_{C}s and 800× non-targeted microbubbles, MB_{O}s. These were all infused at 20 dynes/cm² shear stress 1-way flow on the same 6-lane micro-flow chamber slide. After 45 minutes of incubation, DAPI nuclear staining was performed and excess MBs were washed with HUVECs media at same shear stress. Phase contrast and epifluorescence microscopy was performed in 6 random high power fields. Cells and microbubbles were documented by photomicroscopy and counted as to percent cells with bound MB, and number of MBs per cell. We compared MB_{D}, MB_{C} and non-targeted microbubbles MB_{O}.

Statistical analysis. Values are expressed as mean±S.E.M. Data were analyzed with Prism 5 statistics software (GraphPad Software Inc, CA). Where applicable, nonparametric ANOVA and Dunn's multiple comparison tests or ANOVA and Tukey's multiple pairwise comparison tests were used. For two group comparison, nonparametric Kruskal Wallis test was performed using Prism5 (GraphPad Software Inc, CA).

### Results

DEspR-targeted Molecular Imaging of Carotid Artery. Given the need for detecting vascular disease-associated angiogenesis in carotid artery disease [5,6], DEspR was tested to determine whether it can serve as an endothelial target for contrast enhanced ultrasonographic (CEU)-imaging of pathological angiogenesis in carotid artery disease lesions or vasa vasorum neovascularization. The Tg25 rat model of carotid artery disease was used, comparing 4-month old male Tg25 rats projected to be at midpoint of atherosclerotic disease course [21, 22], with age-matched non-transgenic male littermates. Compared to coronary artery disease, investigation of carotid artery disease provides a tactical experimental system with less movement artifacts.

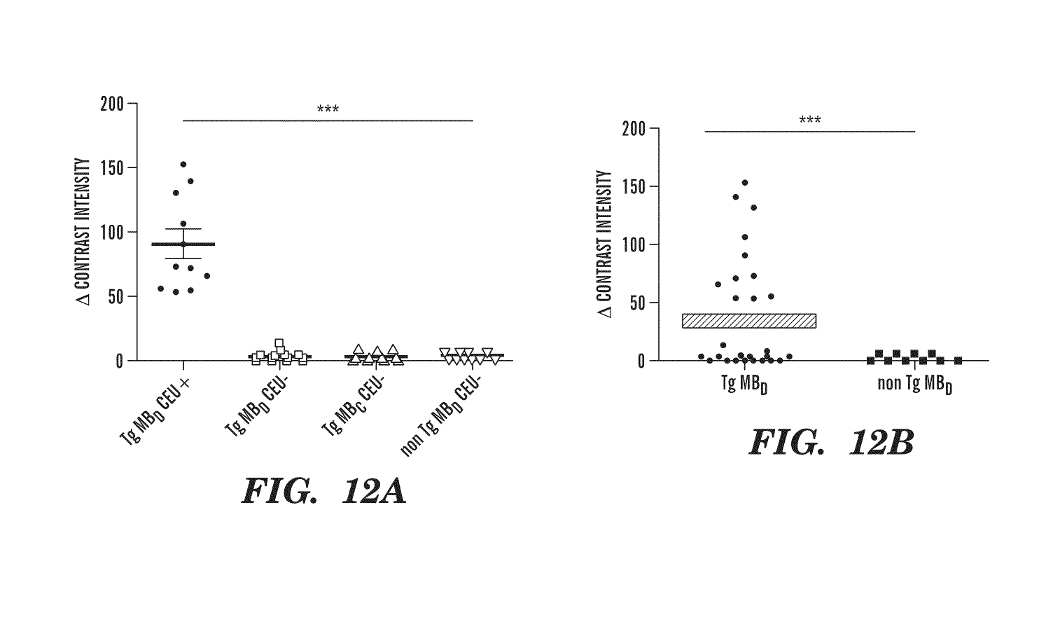

Using the VEVO®770 ultrasound contrast-enhanced imaging system and DEspR-targeted microbubbles (MB_{D}) compared with control isotype-microbubbles (MB_{C}), MB_{D}-specific CEU-positive imaging was detected in different regions-of-interest (ROI) along the common carotid artery (CCA), carotid artery bifurcation, proximal internal and/or external carotid arteries in 7/13 transgenic rats. MB_{D}-specific CEU-positive imaging was defined as stably increased contrast intensity signals detected after circulating microbubbles have cleared, and which decreased upon acoustic destruction. The peak pre-destruction contrast intensity signals and the differences in pre-/post-destruction contrast intensity signals (CIS-differences) were significantly higher in MB_{D}-specific CEU-positive images (Table 2) compared with CEU-imaging observed in isotype MB_{S}-infused rats and in MB_{D}-infused non-transgenic control rats (n=5), with the latter two empirically defining CEU-negative imaging. Notably, of the 7 transgenic rats exhibiting MB_{D}-specific CEU-positive imaging, four exhibited CEU-positive imaging in both carotid arteries, while three exhibited CEU-negative imaging on the contra-lateral carotid artery, suggesting selectivity of MB_{D}-specific CEU-positive imaging and concordant with specificity (Table 2). Moreover, six transgenic rats exhibited CEU-negative imaging with low peak contrast intensity signals, “flat-line” pre-/post-destruction CIS-plot pattern, and minimal CIS-differences (FIGS. 12A, 12B, Table 2) similar to CEU-negative imaging observed in MB_{S}-control rats and in MB_{D}-infused non-transgenic controls.

Altogether, these observations provide compelling evidence that MB_{D}-based CEU-positive images are specific and due to adherent MB_{D}s in said carotid arteries. Statistical analysis by one way analysis of variance (ANOVA) and post-hoc multiple comparison testing establish that the CIS-differences of MB_{D}-specific CEU-positive imaging are significantly higher, P <0.0001, compared to each CEU-negative imaging study group, respectively (Table 2, FIG. 12A). Interestingly, since CEU-positive imaging is detected only in transgenic rats, and with 54% of transgenic rats exhibiting MB_{D}-specific CEU-positive imaging at 4 months of age equivalent to an early-midpoint of the typical model disease course in males [21, 22], average CIS-differences are significantly different (P <0.0001) between transgenic rats and their non-transgenic controls (FIG. 12B). With 7/13 transgenic rats exhibiting CEU-positive imaging, and 6/13 exhibiting CEU-negative imaging upon MB_{D} infusion, a sub-grouping of transgenic rats based on MB_{D} CEU-imaging CIS-differences at the 4-month midpoint of the disease course is apparent (FIG. 12B).

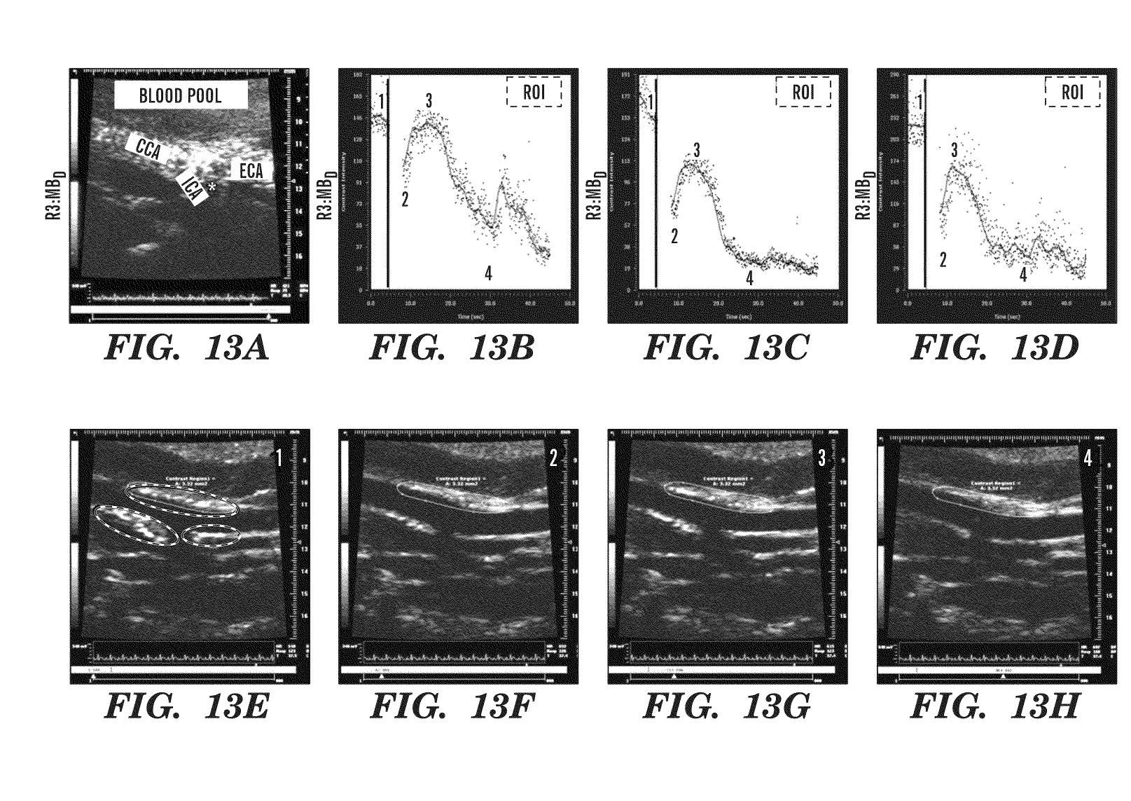

Interestingly, the CIS-plots of three transgenic rats with the highest MB_{D}-specific CIS-differences exhibited the expected post-acoustic destruction drop in signal intensity but had secondary peaks of contrast intensity signals followed subsequently by decline to low/baseline levels (FIGS. 13A-13H). This post-acoustic destruction/disruption pattern is consistent with a particular sequence of microbubble events: microbubble fragmentation accounting for the drop, residual microbubble acoustic stimulation accounting for the secondary peak, followed by acoustically driven diffusion accounting for the subsequent steady decline to baseline levels.

Histological analysis detects MB_{D}-microbubbles on DEspR-positive endothelial cells. Unexpectedly, Masson-trichrome stained histological analysis detected a few microbubbles still attached to endothelial cells or within intimal lesions obtained from R1:MB_{D} rat with CEU-positive imaging shown. Corresponding DEspR-immunostaining on the adjacent serial section confirmed adherence of MB_{D}-microbubbles to DEspR-positive endothelial cells. Immunostaining with isotype antibody confirms specificity of DEspR-positive immunostaining. Altogether, these observations corroborate MB_{D}-binding and specificity of MB_{D}-binding to DEspR-positive endothelium. Survival of PEG-coated Target-ready MicroMarker microbubbles (VisualSonics, Inc., Canada) through PBS-buffered 4% paraformaldehyde fixation, paraffin embedding and deparaffinization parallels our observation that PEG-based biomaterials survive fixation, paraffin embedding, deparaffinization and Masson trichrome staining [24].

Histological analysis of R3:MB_{D} rat shown in FIGS. 13A-13H also detected increased endothelial DEspR-positive expression and luminal endothelial pathology, as well as marked carotid vasa vasoral expansion by neovascularization with DEspR-positive expression in vasa vasorum neovessel. Double-immunofluorescence immunostaining with DEspR and α-smooth muscle actin (αSMA) detected some co-localization of DEspR+αSMA-positive immunostaining in carotid artery vasa vasorum.

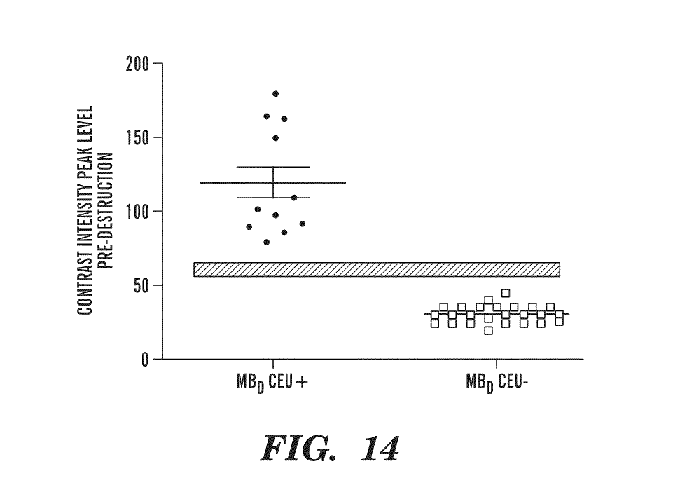

Increased DESPR-expression is associated with DEspR positive molecular imaging. To determine whether increased level and/or area of DEspR-expression is associated with MB_{D}-specific CEU-positive imaging defined by higher CIS-differences (FIG. 12A) and higher pre-destruction CIS-peak levels (FIG. 14), double immunofluorescence-staining was performed with anti-DEspR and anti-α-smooth muscle alpha actin (αSMA) antibodies, the latter serving as a positive control for immunostaining of vascular smooth muscle cells in the media. Serial sections from representative rats were analyzed (n=3/group) with MB_{D}-specific bilateral CEU-positive imaging, MB_{D}-infused bilateral CEU-negative imaging, and with one-sided CEU-positive/CEU-negative imaging. Analysis of immunofluorescence and differential-interference contrast (DIC)-microscopy showed that MB_{D}-specific CEU-positive imaging is associated with DEspR+expression in carotid intimal lesions, vasa vasorum neovascularization and DEspR+expression in vasa vasorum neovessels (Table 2). In contrast, rat carotid arteries exhibiting MB_{D}-CEU-negative molecular imaging were associated with minimal, if any, DEspR+endothelial expression (Table 2). Low levels of αSMA expression in carotid media smooth muscle cells (SMCs) compared with the expanded vasa vasorum were also noted (FIG. 14), due, without wishing to be bound or limited by a theory, most likely to the synthetic state of SMCs in these hypertensive rats, since αSMA expression is deinduced in synthetic or proliferating SMCs [25]. These observations link MB_{D}-specific CEU-positive imaging in this rat model with increased DEspR expression intensity and area in both intimal lesions and vasa vasorum neovessel density.

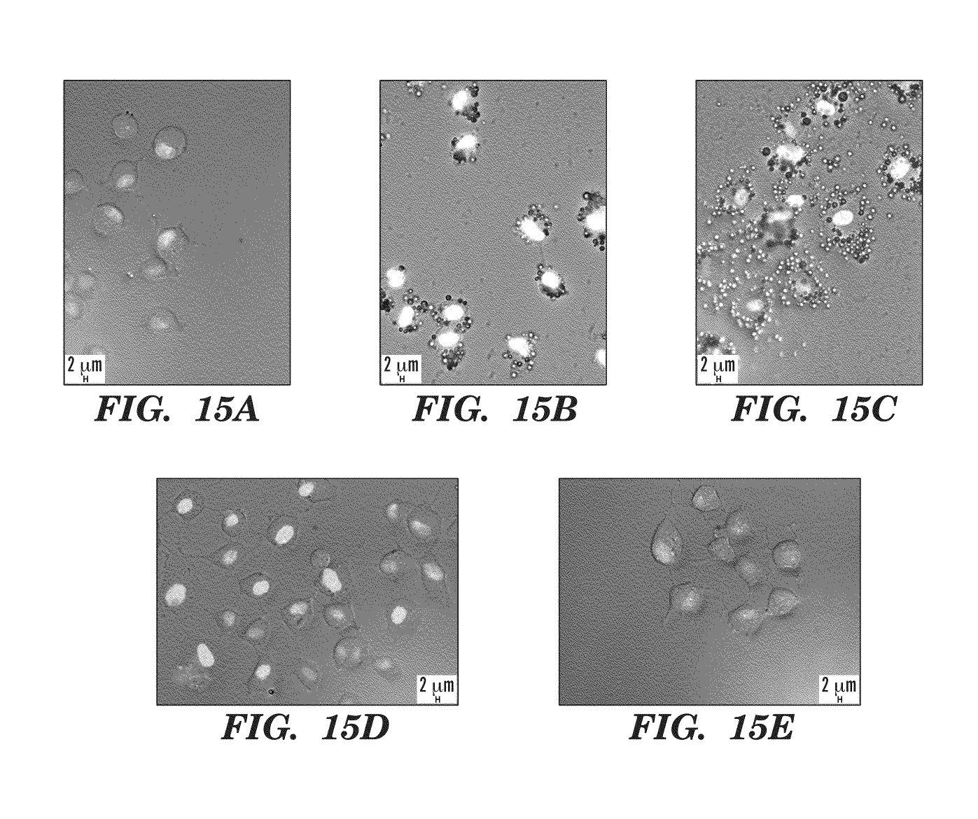

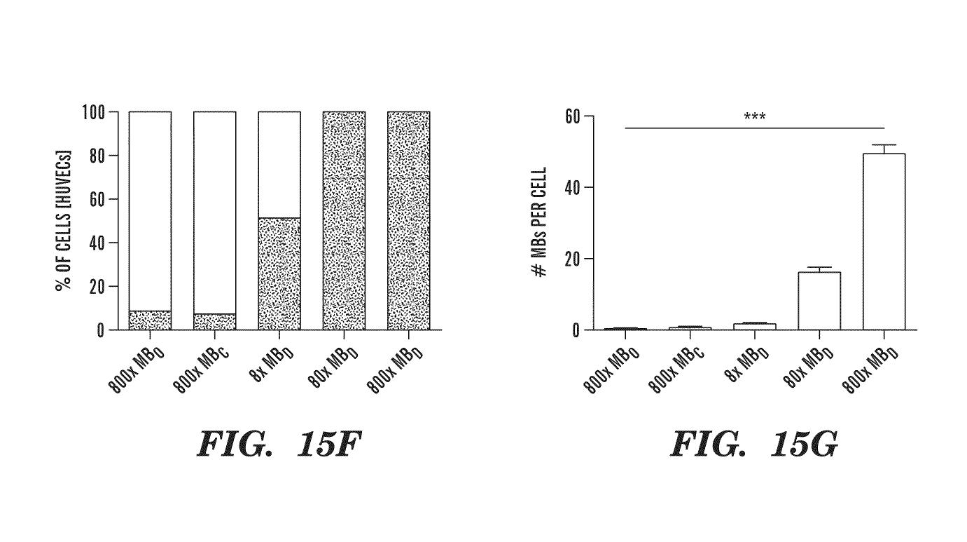

In vitro analysis of dose-response MB_{D}-adherence to DEspR-positive endothelial cells. In order to further dissect MB_{D} interactions with DEspR-positive cells, the dose-response of MB_{D} adherence in vitro was tested. In order to avail of standardized primary cultures of endothelial cells and to gain translational insight into molecular imaging in humans, human umbilical vein endothelial cells (HUVECs) which express DEspR in proliferating and pro-angiogenesis culture conditions as detected by a human-specific anti-DEspR monoclonal antibody were used. Using increasing number of MB_{D}s from 8×, 80×, and 800×MB_{D} to cell ratio, it was observed that HUVECs are increasingly bound by MB_{D}s being 100% bound at 80×MB_{D}:cell ratio (FIGS. 15A-15C), in contrast to 800×MB_{C}s (FIG. 15D) and non-targeted MB_{O}s (FIG. 15E) which bound 6.8% and 8.2% of HUVECs respectively (FIG. 15F). Moreover, analysis of number of MB s bound per cell after a 45-minute incubation and wash at flow rates with aortic-like shear stress of >20 dyne/cm² revealed significant differences in number of MBs bound per cell increasing from 8×, 80× to 800× as follows: 2.3, 17 and 49 MBs/cell, with only 0.6 and 1.1 MB/cell for non-targeted MBs and isotype MB_{C}s (ANOVA P <0.0001). These observations reflect the relative stability and specificity of the MB-cell interaction. Importantly, cell toxicity was not observed upon contact of MB with cells even at high-dose 800×MB_{D}s.

Although VEGFR2-targeted molecular imaging of tumor angiogenesis has been reported [12], previous VEGFR2-targeted molecular imaging of vasa vasorum neovascularization was not successful, along with other vascular adhesion molecule targets, leading authors of these reports to suggest that vasa vasoral flow might be a technical hurdle for target-specific CEU-molecular imaging [9]. Accordingly, the molecular imaging of DEspR-positive endothelial cells in carotid artery disease demonstrated herein (FIGS. 12A-14) provide novel research and diagnostic tools for in vivo molecular imaging of carotid artery disease endothelium and expanded vasa vasorum. Without wishing to be bound or limited by theory, given optimal ultrasound imaging parameters, the likely factors for differential success in target-specific CEU-molecular imaging could be differences in molecular thresholds defined by the level and/or area of expression of the target, and/or in technical thresholds defined by density and size of, as well as flow in target vessel(s). These thresholds must be surpassed concurrently for detectable targeted CEU-positive imaging or molecular imaging. More specifically, the level of DEspR expression, the degree of luminal endothelial pathology, and the density of vasa vasorum neovascularization, along with the larger size of the rat carotid artery disease model used here, comprise factors contributing to successful DEspR-targeted CEU-positive imaging of carotid artery vasa vasorum in the Tg25 rat model of carotid artery disease, in contrast to the negative molecular imaging results targeting VEGFR2 reported for vasa vasorum neovascularization in a carotid artery injury-induced mouse model [9]. Furthermore, differences between CEU-positive transgenic rats from CEU-negative transgenic rats reveal a putative threshold for CIS-differences (FIG. 12B) and pre-disruption CIS-peak levels (FIG. 14). This observed threshold for CEU-positive imaging provides evidence that DEspR-targeted CEU-positive imaging can be a non-invasive biomarker for pathological angiogenesis, and have predictive value for disease progression.

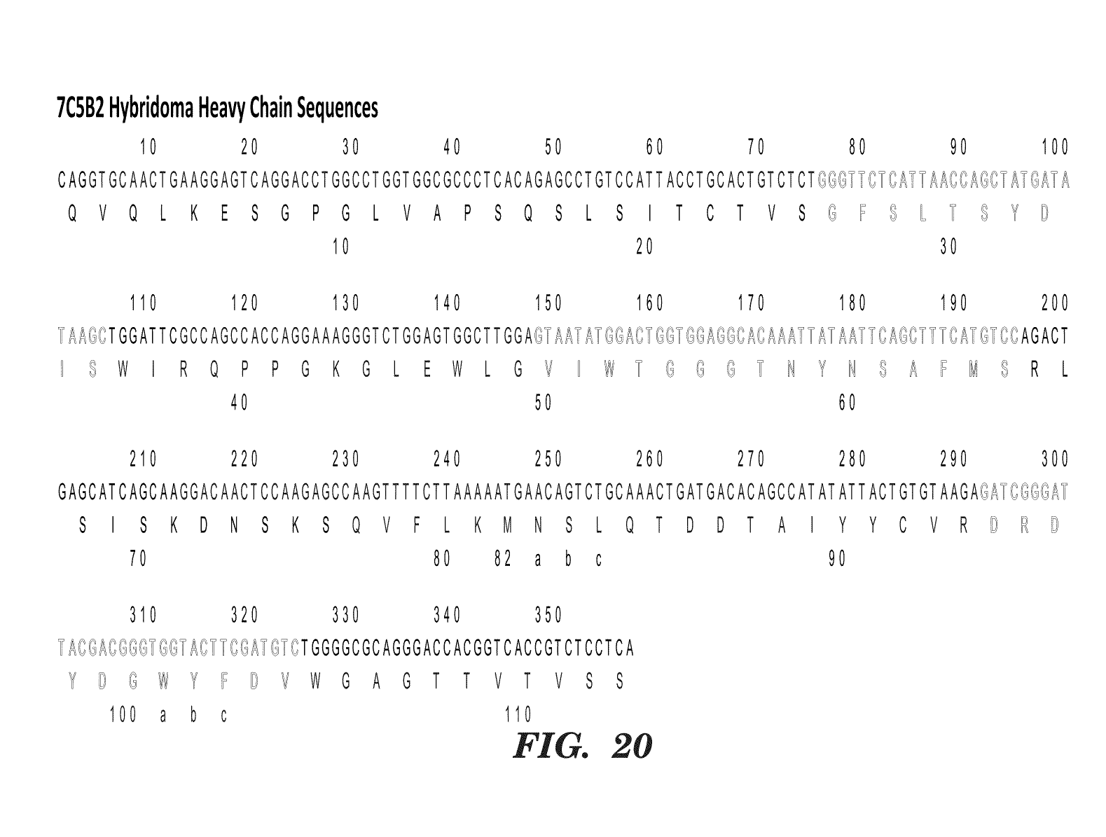

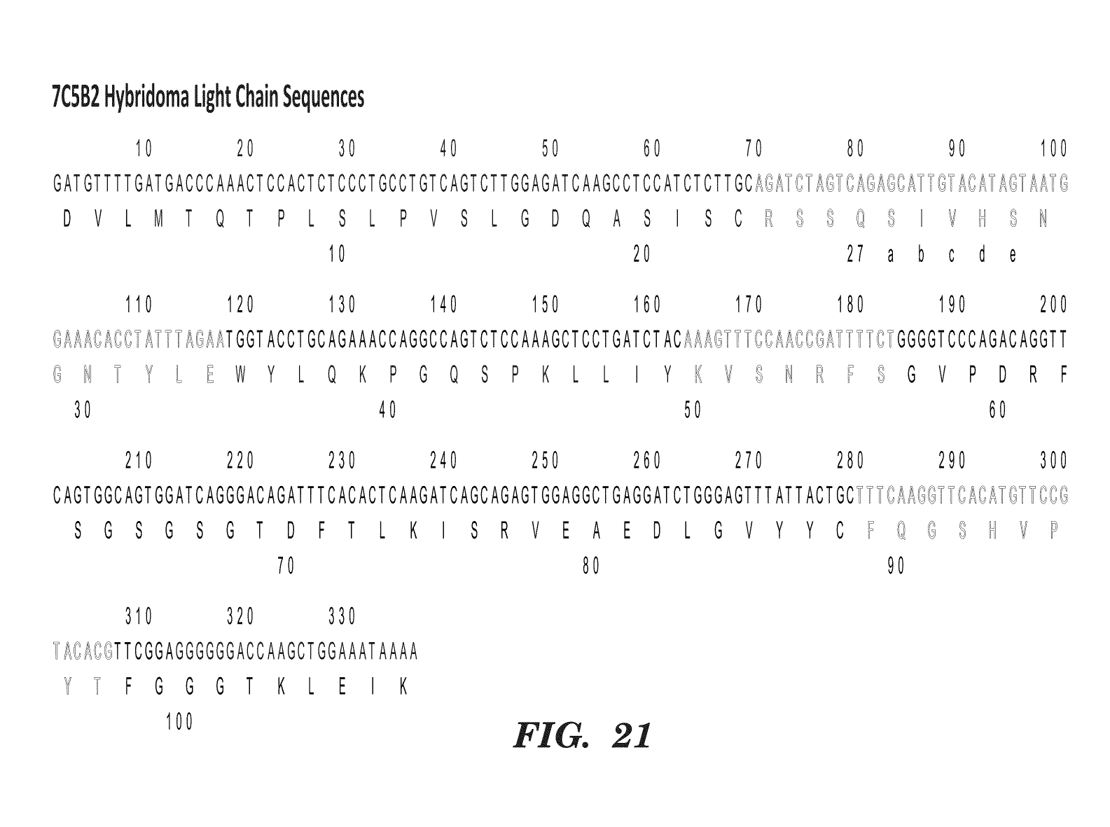

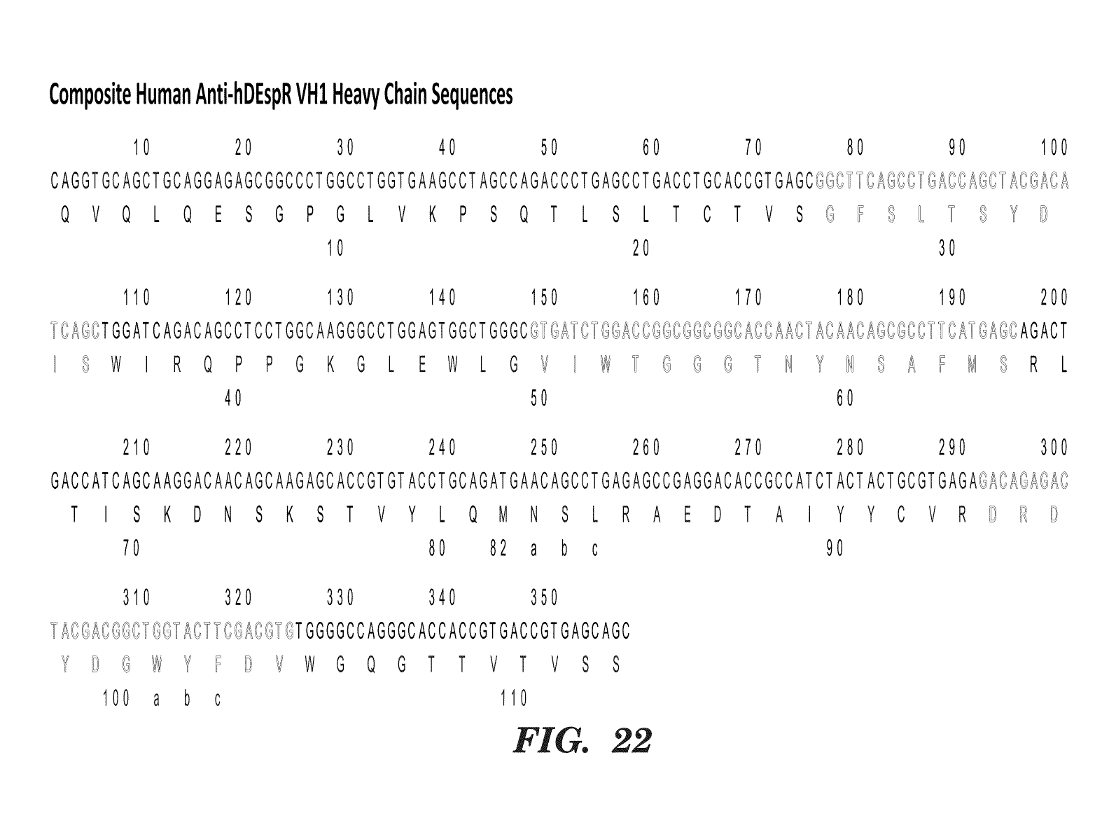

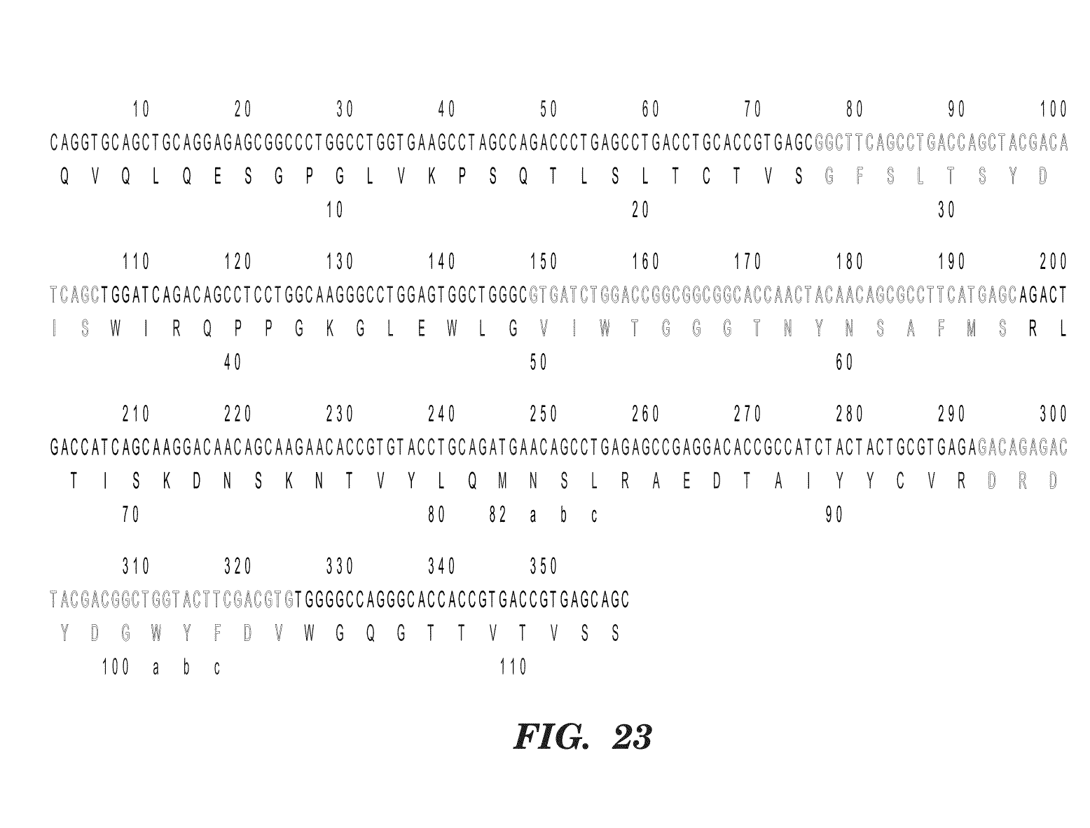

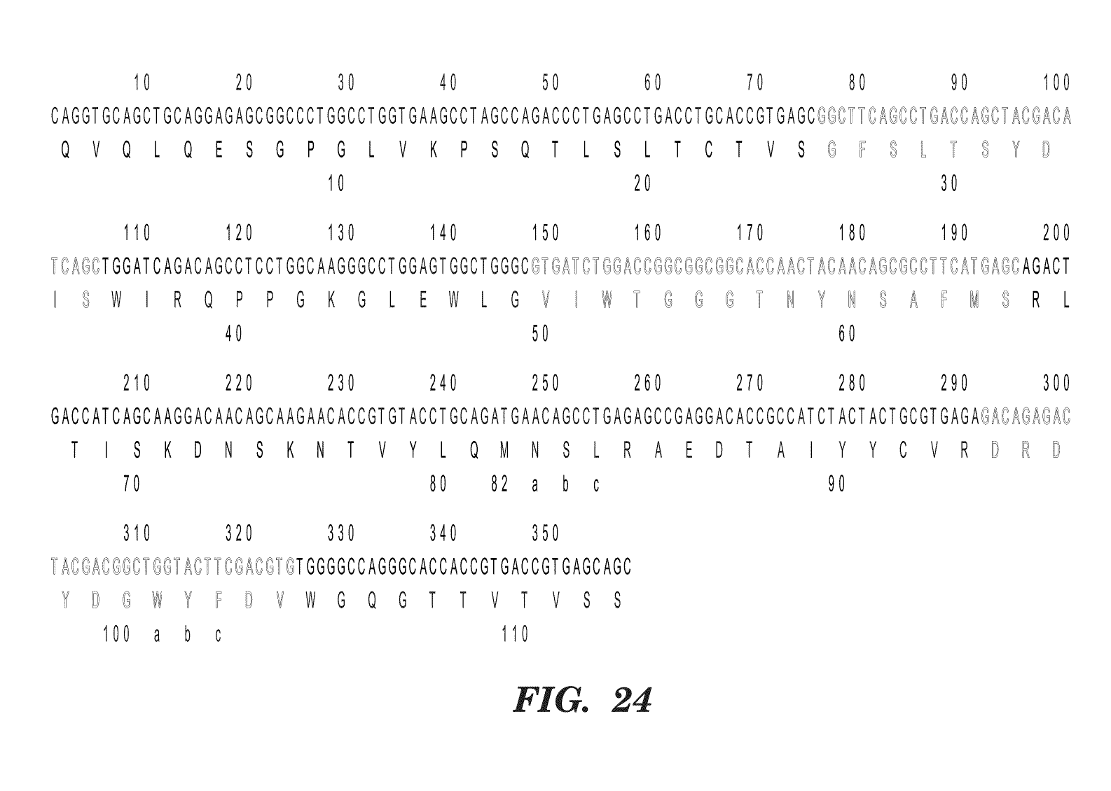

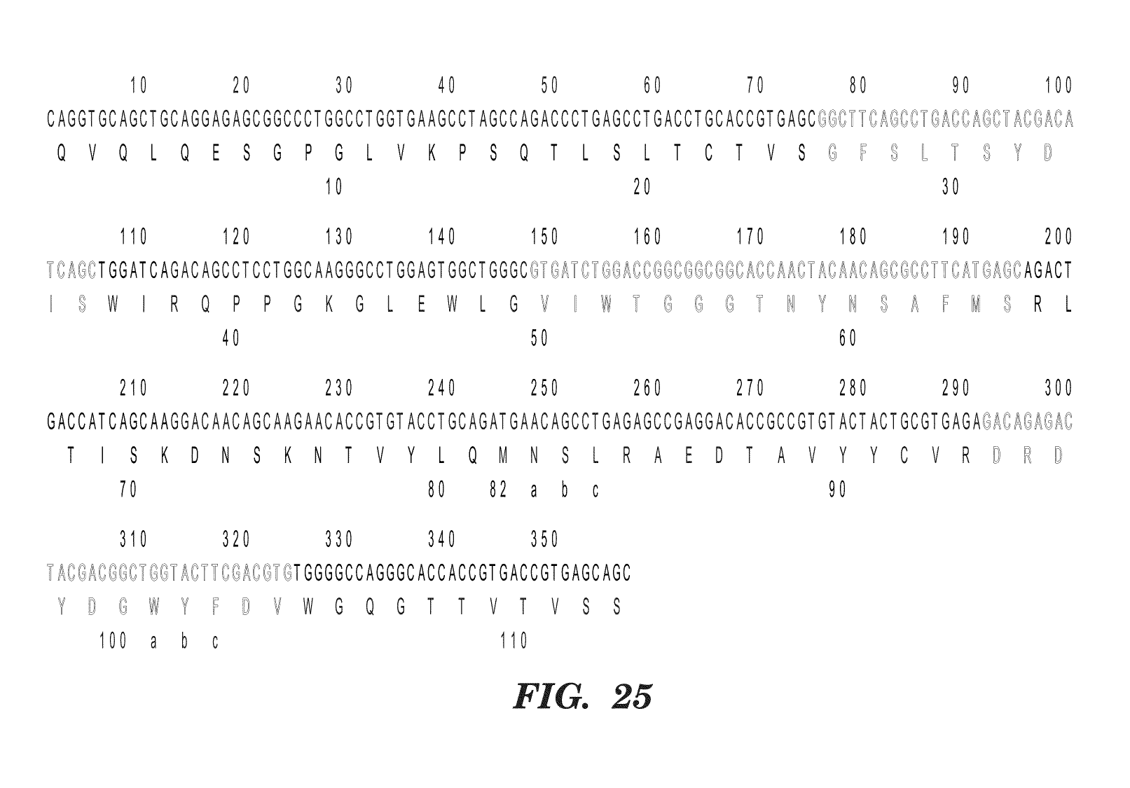

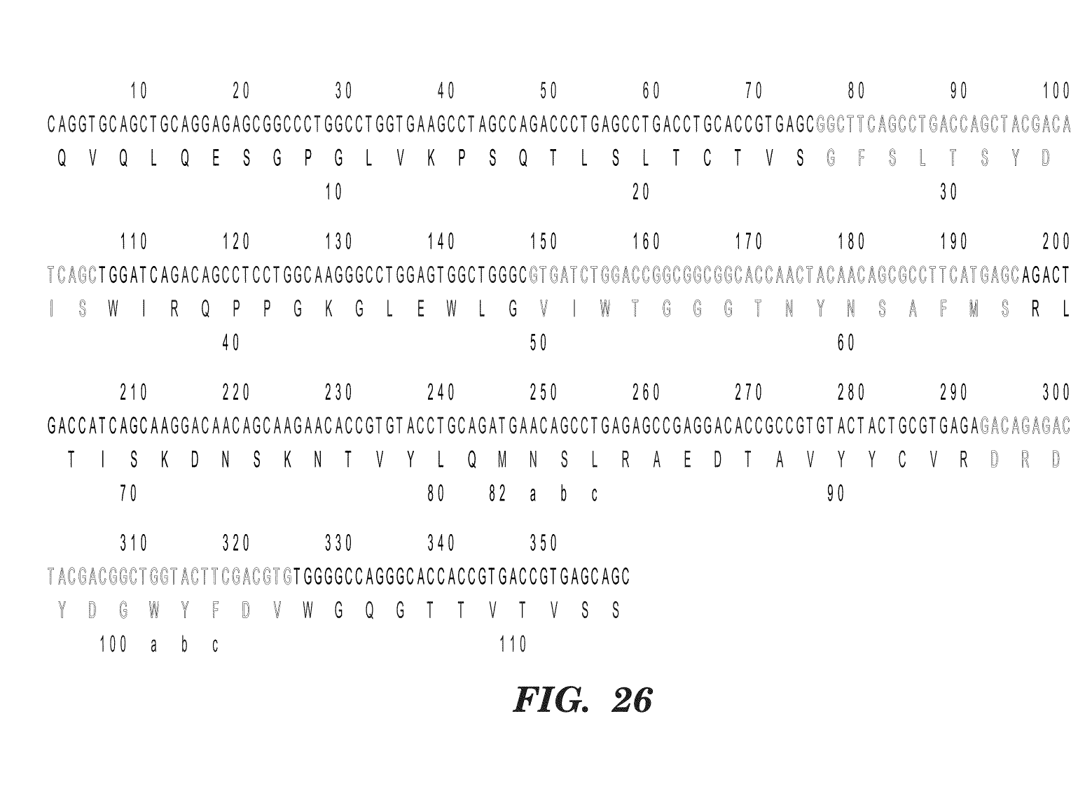

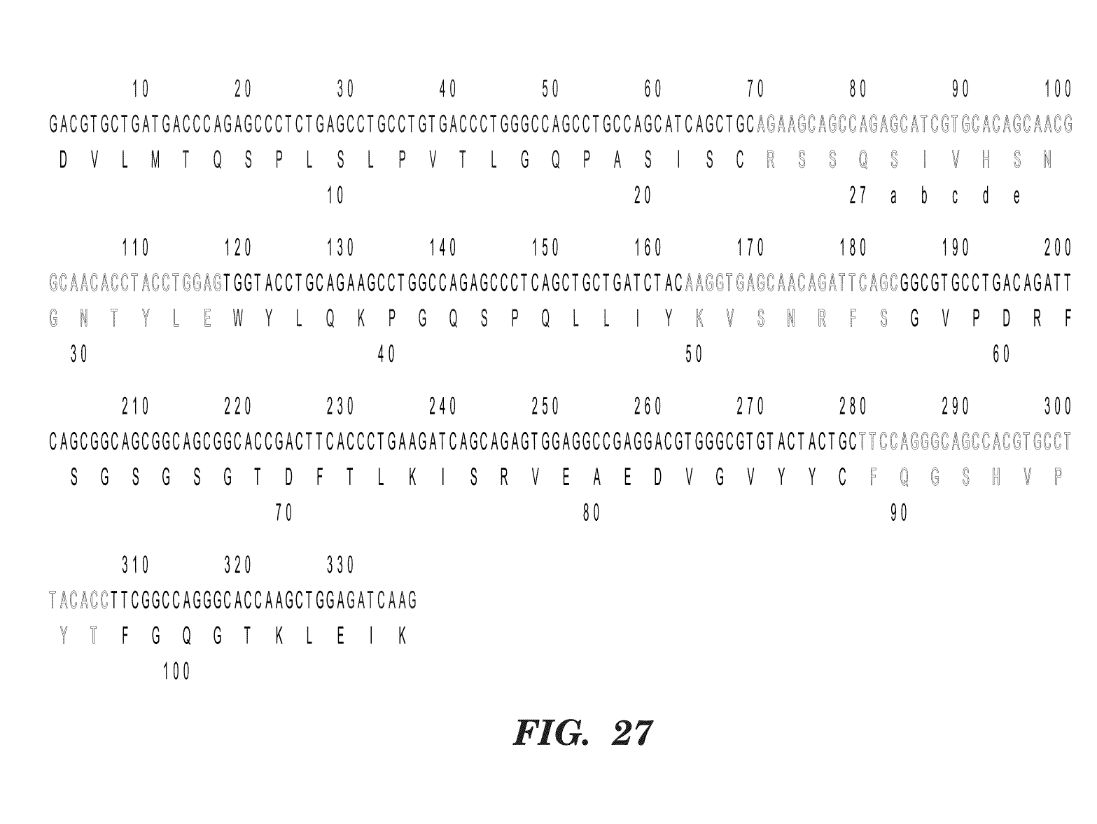

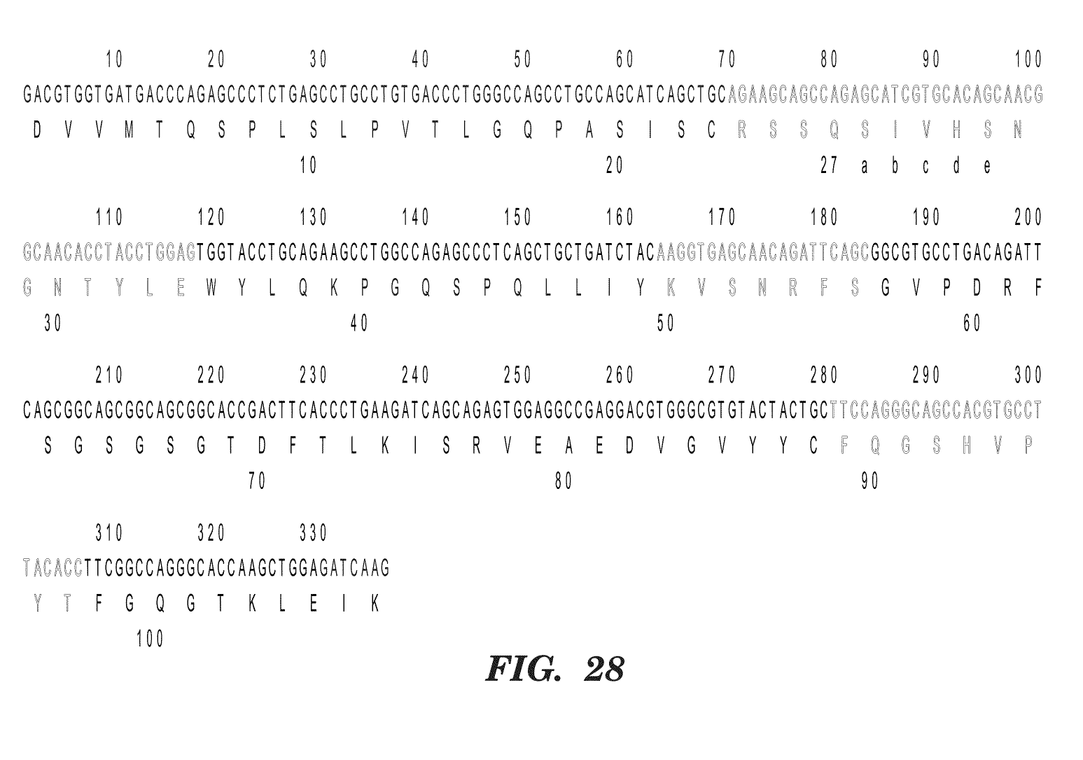

Surpassing the molecular and technical threshold for successful detection of target-specific molecular imaging is concordant with the principle that reflectivity is directly proportional to the concentration of the microbubbles themselves [26]. More specifically, greater DEspR-expression and greater density of DEspR-positive endothelial cells, be it at the lumen or in vasa vasorum, can translate to greater concentration of bound microbubbles in the methods described herein. This in turn, without wishing to be bound or limited by theory, is expected to translate to greater reflectivity and detection levels since microbubble-cell binding does not dampen microbubble reflectivity in contrast to leukocyte engulfment of microbubble [27]. After clearance of most circulating microbubbles and prior to acoustic disruption, stable binding of target-specific microbubbles exhibits a relatively stable contrast-intensity level that is significantly greater than negative or background contrast-intensity (FIG. 20D, ANOVA P <0.0001). Since high-frequency imaging can induce microbubble fragmentation or gas diffusion per se, a slight decline could also be observed prior to acoustic disruption, without wishing to be bound or limited by theory. However, upon acoustic disruption a drop in contrast-intensity due to fragmentation is observed to confirm microbubble binding (FIGS. 12A-12B). Acoustic fragmentation may not be complete due, without wishing to be bound or limited by theory, to microbubble interaction in high-density ROIs which could dampen microbubble resonance [28], or from inability of microbubbles within microvessels to reach 10-fold diameter-fluctuation that underlies acoustic fragmentation [29]. Furthermore, incomplete fragmentation with gas release and relatively low flow, as would be expected in vasa vasorum compared to carotid artery lumen, without wishing to be bound or limited by theory, could account for the secondary peak observed in rat-R3 followed by slow decline back to baseline levels. The secondary peak is likely not due to refill because at this experimental time point there is minimal, if any, circulating microbubbles (FIGS. 12A-12B, 13A-13H). The fact that rat-R3 reached higher contrast-intensity levels than rat-R1 suggests greater microbubble concentration, which can also dampen acoustic destruction due to inter-microbubble interactions [28]. Notably, while acoustic fragmentation corroborates microbubble binding, the pattern of acoustic fragmentation or diffusion can also provide further insight into microbubble concentration, as well as binding site vessel-caliber and flow. This provides a novel, alternative molecular imaging paradigm to that reported for mouse aortic root atherosclerosis [30]. While CEU-imaging in the current set-up is successful, in other embodiments, non-linear imaging of adherent microbubbles can be used to provide greater sensitivity and/or improved quantitation as observed for intravascular ultrasound for vasa vasorum flow imaging [31].

The detection of dose-dependent increase in % cells targeted by MB_{D}s and dose-dependent increase in number of MB s per cell (FIGS. 15A-15G), gives insight into the stable interaction, kinetics, specificity and non-toxicity of DEspR-targeted MB-cell interactions. More importantly, given that in vitro studies were performed using human endothelial cells and human-specific anti-DEspR monoclonal antibody for targeting, that MB-cell coupling withstood a high shear stress wash after 45 minutes and did not elicit cell toxicity on contact, these in vitro observations of MB_{D}-cell interactions demonstrate DEspR-targeted molecular imaging of pathological angiogenesis as a useful therapeutic and diagnostic tool.

Altogether, comparative analysis of molecular imaging contrast-intensity levels, histological confirmation of microbubble-to-endothelium binding, immunostaining confirmation that DEspR-positive molecular imaging is associated with DEspR-positive endothelial cell expression, and concordant patterns of bound microbubble behavior after acoustic destruction, demonstrate that target-specific molecular imaging of carotid endothelium and vasa vasorum neovascularization in carotid artery disease rat model is feasible using the methods and reagents described herein that target DEspR. The identification of DEspR as a successful target for in vivo molecular imaging of vasa vasorum neovascularization and carotid artery disease lesions can facilitate the longitudinal study of vasa vasorum neovascularization and endothelial changes in carotid artery disease progression in animal models. Along with the in vitro observations of MB_{D}-HUVECs stable binding, the data demonstrate the use of molecular imaging techniques described herein in the earlier detection of pathophysiological changes in cardiovascular disease for estimations of risk for disease progression and complications.

### REFERENCES

1. Ruiz-Opazo, N.; Hirayama, K.; Akimoto, K.; Herrera, V. L M. Molecular characterization of a dual endothelin-1/angiotensin II receptor. Mol. Med. 4:96-108, 1998.
2. Herrera, V. L. M.; Ponce, L. R. B.; Bagamasbad, P. D.; VanPelt, B. D.; Didishvili, T.; Ruiz-Opazo, N. Embryonic lethality in Dear gene-deficient mice: new player in angiogenesis. Physiol. Genomics. 23:257-268, 2005.
3. Ferrara, N.; Carver-Moore, K.; Chen, H.; et al. Heterozygous embryonic lethality induced by targeted inactivation of the VEGF gene. Nature. 380:439-442, 1996.
4. Carmeliet, P,; Ferreira, V.; Breir, G.; et al. Abnormal blood vessel development and lethality in embryos lacking a single VEGF allele. Nature. 380:435-439, 1996.
5. Dunmore, B. J.; McCarthy, M. J.; Naylor, A. R.; Brindle, N. P. Carotid plaque instability and ischemic symptoms are linked to immaturity of microvessels within plaques. J. Vasc. Surg. 45:155-159, 2007.
6. Giannoni, M. F.; Vicenzini, E.; Citone, M.; et al. Contrast carotid ultrasound for the detection of unstable plaques with neoangiogenesis: a pilot study. Eur. J. Vasc. Endovasc. Surg. 2009; doi: 10.10.16/j.ejvs0.2008.12.028.
7. Vincenzini, E.; Giannoni, M. F.; Benedetti-Valentini, F.; Lenzi, G. L. Imaging of carotid plaque angiogenesis. Cerbrovasc. Dis. 27(Supp12):48-54, 2009.
8. Kaufmann, B. A.; Lindner, J. R. Molecular imaging with targeted contrast ultrasound. Current Opinion in Biotech. 18:11-16, 2007.
9. Lindner, J. R. Molecular imaging of cardiovascular disease with contrast-enhanced ultrasonography. Nat. Rev. Cardiol. 6:475-481, 2009.
10. Ellegala, D. B.; Leong-Poi, H.; Carpenter, J. E. et al. Imaging tumor angiogenesis with contrast ultrasound and microbubbles targeted to alpha(v) beta3. Circulation. 108:336-341, 2003.
11. Leong-Poi, H.; Christiansen, J.; Heppner, P.; et al. Assessment of endogenous and therapeutic arteriogenesis by contrast ultrasound molecular imaging of integrin expression. Circulation. 111:3248-3254, 2005.
12. Willmann, J. H.; Paulmurugan, R.; Chen, K.; et al. US imaging of tumor angiogenesis with microbubbles targeted to vascular endothelial growth factor receptor type 2 in mice. Radiology. 246:508-518, 2008.
13. Weller, G. E.; Lu, E.; Csikari, M. M.; et al. Ultrasound imaging of acute cardiac transplant rejection with microbubbles targeted to intercellular adhesion molecule-1. Circulation. 108:218-224, 2003.
14. Hauff, P.; Reinhardt, M.; Briel, A.; Debus, N.; Schirner, M. Molecular targeting of lymph nodes with L-selectin ligand-specific US contrast agent: a feasibility study in mice and dogs. Radiology. 231:667-673, 2004.
15. Kaufmann, B. A.; Sanders, J. M.; Davis, C. et al. Molecular imaging of inflammation in atherosclerosis with targeted ultrasound detection of vascular cell adhesion molecule-1. Circulation. 116:276-284, 2007.
16. Christiansen, J. P.; Leong-Poi, H.; Klibanov, A. L.; Kaul, S.; Lindner, J. R. Noninvasive imaging of myocardial reperfusion injury using leukocyte-targeted contrast echocardiography. Circulation. 105:1764-1767, 2002.
17. Villanueva, F. S.; Wagner, W. R. Ultrasound molecular imaging of cardiovascular disease. Nat. Clin. Pract. Cardiovasc. Med. 5:S26-S32, 2008.
18. Schumann, P. A.; Christiansen, J. P.; Quigley, R. M. al. Targeted-microbubble binding selectively to GPIIb/IIIa receptors of platelet thrombi. Invest. Radiol. 37:587-593, 2002.
19. Hamilton, A.; Huang, S. L.; Warninck, D. et al. Left ventricular thrombus enhancement after intravenous injection of echogenic immunoliposomes: studies in a new experimental model. Circulation. 105:2772-2778, 2002.
20. Lee, S.; Can, C. L.; Belcik, T. A. et al. Contrast-enhanced ultrasound characterization of inflammation and vasa vasoral proliferation caused by mural hemorrhage and platelet deposition. Circulation. 118:S644, 2008 (Abstract 1074).
21. Decano, J. L.; Viereck, J. C.; McKee, A. C.; Hamilton, J. A.; Ruiz-Opazo, N.; Herrera V. L. M. Early-life sodium exposure unmasks susceptibility to stroke in hyperlipidemic, hypertensive heterozygous Tg25 rats transgenic for human cholesteryl ester transfer protein. Circulation. 119:1501-9, 2009.
22. Herrera, V. L. M.; Tsikoudakis, A.; Didishvili, T.; et al. Analysis of gender-specific atherosclerosis susceptibility in transgenic[hCETP]25^{DS} rat model. Atherosclerosis. 177:9-18, 2004.
23. Loveless, M. E.; Li, X.; Huamani, J.; et al. A method for assessing the microvasculature in a murine tumor model using contrast-enhanced ultrasonography. J. Ultrasound Med. 27:1699-1709, 2008.
24. Herrera, V. L.; Viereck, J. C.; Lopez-Guerra, G. et al. 11.7 Tesla magnetic resonance microimaging of laryngeal tissue architecture. Laryngoscope. 119:2187-94, 2009.
25. Blindt, R.; Vogt, F.; Lamby, D.; et al. Characterization of differential gene expression in quiescent and invasive human arterial smooth muscle cells. J. Vasc. Res. 39:340-352, 2002.
26. Calliada, F.; Campani, R.; Bottinelli, O.; Bozzini, A.; Sommaruga, M. G. Ultrasound contrast agents: basic principles. Eur. J. Radiol. 27Suppl 2:S157-160, 1998.
27. Lankford, M.; Behm, C. Z.; Yeh, J.; Klibanov, A. L.; Robinson, P.; Lindner, J. R. Effect of microbubble ligation to cells on ultrasound signal enhancement: implications for targeted imaging. Invest. Radiol. 41:721-728, 2006.
28. Yasui, K.; Lee, J.; Tuziuti, T.; Towata, A.; Kozuka, T.; Iida, Y. Influence of the bubble-bubble interaction on destruction of encapsulated microbubbles under ultrasound. J. Acoust. Soc. Am. 126:973-982, 2009.
29. Chomas, J. E.; Dayton, P.; Allen, J.; Morgan, K.; Ferrara, K. W. Mechanisms of contrast agent destruction. IEEE Trans. Ultrason. Ferroelectr. Freq. Control. 48:232-248, 2001.
30. Kaufmann, B. A.; Carr, C. L.; Belcik, T. et al. Molecular imaging of the initial inflammatory response in atherosclerosis. Arterioscler. Thromb. Vasc. Biol. 30:54-59, 2010.
31. Goertz, D. E., Frijlink, M. E., Tempel, D., et al. Subharmonic contrast intravascular ultrasound for vasa vasorum imaging. Ultrasound Med. Biol. 33:1859-1872, 2007.
32. Kaufmann, B. A. Ultrasound molecular imaging of atherosclerosis. Cardiovasc. Research. 83:617-625, 2009.

TABLE 2
DEspR-targeted molecular imaging in transgenic rat model
of carotid artery disease

| | | | Non- |
|---|---|---|---|
| Rat groups: 4 m-old male | Tg 25+ | | transgenic |
| MB_{D} Contrast enhanced | CEU(+) | CEU(−) | CEU(−) |
| image | | | |
| # rats: both carotid arteries | 4 | 6 | 5 |
| # rats: one carotid artery | 3* | 3* | — |
| Contrast intensity signal Δ | | | |
| MB_{D} (n = 18 rats) | 89.96 ± 11.0*** | 2.2 ± 0.9 | 2.0 ± 0.8 |
| MB_{C} (n = 8 rats) | 1.9 ± 0.7 | ND | ND |
| Histopathology: | | | |
| Intimal lesions, plaque | (+) | +/− | (−) |
| Vasa vasorum expansion | (+) | +/− | (−) |
| Immunostaining: | | | |
| DEspR | (+): in vasa | +/− | (−) |
| | vasorum, initimal | | |
| | lesions | | |

Values are group means ± sem;
#, number;
Δ, delta or difference;
(+), present;
(−), absent;
+/−, low to no expression;
*, same 3 rats;
***, ANOVA and Tukey's multiple pairwise comparison P < 0.0001.
CAD, carotid artery disease;
m, month;
MB_{D}, DEspR-targeted microbubble;
MB_{C}, isotype-targeted microbubble.

### Example 3

### Dual endothelin-1/VEGFsp Receptor (DEspR) in Cancer: Target for Dual Anti-Angiogenesis/Anti-Tumor Cell Invasiveness Therapy

The development of intrinsic and extrinsic resistance to current anti-VEGF/VEGFR2 therapies have been observed. As described herein, DEspR expression is found to be increased in primary and metastatic tumor αSMA-positive and αSMA-negative vascular endothelium, and in tumor cell- and nuclear-membranes of different human cancer tissue types and cell lines. Further, DEspR-inhibition using the human-specific anti-DEspR antibody treatments described herein decreased human endothelial cell angiogenesis and tumor cell invasiveness. Further, it was found that ligand-specific DEspR signaling-profiles are distinct from VEGFNEGFR2's. Accordingly, described herein are data demonstating targeting of DEsPR for dual tumor-cell and endothelial deliveries, and for dual anti-angiogenesis/anti-invasiveness therapies.

### Introduction

Although the critical role of the angiogenic switch in cancer pathogenesis has been recognized [1], anti-angiogenesis therapies directed at vascular endothelial growth factor and/or its receptor, VEGF/VEGFR2-centric anti-angiogenesis therapies, alone or in combination with other anti-cancer therapies, have not attained the hoped-for treatment goal of long-term efficacy such that cancer is reduced to a dormant, chronic manageable disease [2-5]. Cumulative observations have shows that all three FDA-approved VEGF pathway inhibitors (anti-VEGF bevacizumab or Avastin, AntiVEGFR2 sunitinib, and sorafanib) result in significant but transitory improvements in the form of tumor stasis or shrinkage, and only for certain cancers despite most, if not all cancer types exhibiting pathological angiogenesis[2,6]. Moreover, while anti-VEGF pathway therapies have reduced primary tumor growth and metastasis in preclinical studies [7], recent mouse tumor model studies report that sunitinib and an anti-mouseVEGFR2 antibody, DC101, increased metastasis of tumor cells despite inhibition of primary tumor growth and increased overall survival in some cases [8,9]. Cumulative observations implicate several mechanisms of intrinsic and evasive resistance, such as, without wishing to be bound or limited by theories, pre-existing multiplicity of redundant pro-angiogenic signals; upregulation of alternative pro-angiogenic pathways, recruitment of bone marrow-derived pro-angiogenic cells, increased pericyte coverage for the tumor vasculature obviating the need for VEGF signaling, and invasive and metastatic co-option of normal vessels without requisite angiogenesis [2-5]. Additionally, 10-fold increase in VEGF levels have been detected upon bevacizumab anti-VEGF therapy in humans [10] and upon anti-VEGFR2 ab-therapy in mice [11], which could, without wishing to be bound or limited by a theory, contribute to evasive resistance.

Both VEGF and VEGFsp (vascular endothelial growth factor signal peptide) originate from the same propeptide, and a 10-fold ‘rebound’ increase in VEGF could, without wishing to be bound or limited by a theory, also result in a concomitant 10-fold increase in VEGFsp, thus resulting in a 10-fold increase in VEGFsp's post-cleavage function of activating its receptor, the dual endothelin1/VEGFsp receptor or DEspR, formerly called Dear and deposited in GenBank as Dear [12]. DEspR knockout mouse exhibits arrested vasculogenesis and absent angiogenesis resulting in E10.5-E12.5 day embryonic lethality [13]. Concordantly, DEspR-haploinsufficiency resulted in decreased syngeneic melanoma tumor growth, and anti-DEspR antibody inhibition decreased tumor growth and tumor angiogenesis in rats with irradiation-induced mammary tumors [13]. Furthermore, DEspR's other ligand is endothelin-1 (ET1) [12], and all other known ET1 receptors, ETa and ETb, do not exhibit an embryonic lethal angiogenic phenotype in their respective knockout mouse models [14,15, 16.].

Described herein are novel anti-angiogenic strategies using anti-human DEspR ab-inhibition and characterizing the murine precursor of an anti-DEspR antibody therapeutic. It was found that DEspR is upregulated in some solid tumor cells and tumor vascular endothelium, and that human-specific anti-DEspR polyclonal and monoclonal antibodies inhibit human endothelial cell tube formation and tumor cell invasiveness in vitro, and that DEspR utilizes ligand-specific signaling pathways known to mediate angiogenesis and cancer cell invasiveness.

### Materials and Methods

Cell lines and antibody development MDA-MB-231, MDA-MB-468, and PANC-1 cells were obtained from American Type Culture Collection (Rockville, Md.). MDA-MB-468 and -231 cells were maintained in DMEM media (Sigma Chemical, St. Louis, Mo.) supplemented with 10% FBS, L-glutamine, penicillin, and streptomycin (GPS). PANC-1 cells were maintained in DMEM (Sigma Chemical, St. Louis, Mo.) with high glucose, 10% FBS and GPS. Human umbilical vein endothelial cells, HUVECs, were obtained from Cascade Biologics, Inc., and maintained in Endothelial Growth Media-2 (EGM-2) containing 2% FBS and GPS. Monoclonal antibody development was custom performed by ProMab Biotechnologies, Inc (Richmond, Calif.) using a nine amino-acid DEspR NH₂-terminal peptide, M₁TMFKGSNE₉ of hDEspR as antigen. Screening of hybridoma supernatants and initial characterization of candidate monoclonal antibodies were performed by ELISA using free hDEspR-antigenic peptide as antigen.

Monoclonal antibody characterization by ELISA and Western blot analysis. The M₁TMFKGSNE₉ antigenic peptide was coated directly on wells of a microtiter plate. Appropriate dilutions of primary antibodies were incubated at 37° C. for 1 hr. The wells were then incubated with HRP labeled anti-IgG (SIGMA cat#A0168) at 1:9000 at 37° C. for 1 hr. The reactions were visualized by the addition of 3,3′5,5′-tetramethylbenzidine substrate (incubation at 37° C. for 10 min) and read spectrophotometrically at 450 nm. Western blot analysis was done as described [17] using equal amounts of whole cell protein extract (40 μg) from Cos1 cell transfectants stably expressing hDEspR[17] and corresponding candidate monoclonal antibodies raised against hDEspR specific synthetic peptide. Immunoreactive hDEspR (10 kDa polypeptide) was detected by chemiluminescence using the ECL Western Detection kit (GE Healthcare).

HUVEC tube formation assay for angiogenesis. Validated 2^{nd} passage human umbilical vein endothelial cells—HUVECs (Cascade Biologics, Oregon) were obtained and cultured until the 4th passage and were then harvested at 80% confluence using mild trypsinization. The cell pellet was then washed twice in serum free media (basal media) containing M-200 (Cascade Biologics, Oregon) 1 μg/ml hydrocortisone, 10 ng/ml EGF, 3 ng/ml bFGF and 10 μg/ml heparin. Cells were then resuspended in this serum free media and seeded at 20,000 cells per well (100 μL) onto a 96 well plate Angiogenesis System: Endothelial Cell Tube Formation MATRIGEL™ Matrix (BD Biosciences, MA). Different angiogenic and anti-angiogenic conditions were assayed in quadruplicate as indicated using basal media alone or with one or more of the following: 2% FBS, 20 nM VEGF, 20 nM VEGFsp, 20 nM ET1. Antibodies used for inhibition were all affinity purified and used in the following concentrations: 500 nM anti-hDEspR polyclonal antibody (Pab), 500 nM anti-hDEspR 7C5B2 monoclonal antibody (Mab), 500 nM anti-VEGFsp Pab, and for corresponding isotype controls either 500 nM preimmune IgG (75 μg/ml) for Pab, and 500 nM IgG2b for anti-hDEspR Mab. Different experimental conditions were tested in quadruplicate as follows: basal media alone (BM), BM with 2% FBS; BM with 20 nM VEGF; BM with 20 nM VEGFsp; BM with 20 nM ET1; BM with 20 nM VEGF and 500 nM (75 m/ml) pre-immune IgG; BM with 20 nM VEGF and 500 nM anti-VEGFsp; BM with 20 nM VEGF and 500 nM anti-hDEspR; BM with 20 nM VEGFsp and 500 nM anti-hDEspR; BM with 20 nM ET1 and 500 nM anti-hDEspR; BM with 2% FBS and 500 nM anti-VEGFsp; and BM with 2% FBS plus 500 nM anti-hDEspR. In other experiments increasing concentrations of anti-hDEspR 7C5B2 mAb (0.05-500 nM) were tested. HUVECs were then incubated in different conditions as specified at 37° C. for 16 hours; after which, resulting angiogenic tube formations were viewed under the microscope and images of ˜70% of the well (central parts) were taken for analysis. Various parameters were measured for each angiogenic condition using ImageJ (on the world wide web at rsb.info.nih.gov/ij/) namely total tube length, average tube length, average tube thickness, number of branch points defined as cluster of cells possessing tube-like extensions measuring more than 2× the length of the cell aggregates, number of connections defined as 3 or more connections between tube-like structures in series or parallel and number of closed polygons bounded by the tubular structures.

Invasion assay. MDA-MB-468 and PANC-1 cell invasion assays were performed as described [18] using the BD Bio-Coat MATRIGEL invasion assay system (BD Biosciences, Franklin Lakes, N.J.). MDA-MB-468- and PANC-1 cells were suspended in growth media and seeded onto pre-coated transwell chambers (3×10⁴ cells/well). The transwell chambers were then placed into 24-well plates, to which basal medium only or basal medium containing various concentration of antibodies were added. Cells were incubated for 16 hr and the invading cells were fixed and stained with Diff-Quick stain. The number of invading cells per well were counted under the microscope. Each condition was assessed in four replicates.

Immunostaining of tumor tissue arrays and tumor cells. Human cancer cell line-array DEspR immunostaining was custom-performed by Pantomics, Inc. using our in-house polyclonal human-specific anti-DEspR antibody. Tumor tissue arrays were obtained from Pantomics, Inc. and immunostained for DEspR using polyclonal and monoclonal anti-hDEspR antibodies at 1:20 after demonstration of concentration-dependent immunostaining 1:10, 1:50, 1:100. Deoxyaminobenzidine immunostaining was done using the polyclonal antibody as described [13]. Double immunofluorescence staining was done on deparaffinized sections via the following steps: antigen retrieval, treatment to reduce background, blocking, incubation with primary antibody at 4° C. overnight, secondary antibody incubation overnight at 4° C. with AlexaFluor 568 goat anti-mouse IgG and AlexaFluor 488 goat anti-rabbit IgG, washing, and mounting using Prolong Gold with DAPI (Invitrogen). Negative controls were run using rabbit-isotype antibody for anti-rat DEspR antibody. A Zeiss Axioskop2plus microscope was used for fluorescence imaging and photomicroscopy.

Multiplex analysis of signaling proteins by Ab-microarray. Analysis of ligand-dependent modulation of different signaling pathways by DEspR was custom performed by Kinexus Corp. (Kinexus, Canada) utilizing the KINEX™ Antibody Microarray System spanning 506 phosphoprotein-specific antibodies in duplicates or multiple replicates, as well as 740 pan-specific antibodies of signaling molecules. The effects of ET1- and VEGFsp-DEspR activation were analyzed on multiplex signaling pathways after 30 minutes of ligand-treatment (ET1, 10 nM; VEGFsp, 10 nM), compared with the respective non-activated DEspR in non-treated controls, using Cos1-hDEspR permanent cell transfectants. All fluorescent signals were normalized to background. Data are presented as percentage change from control (% CFC), or change detected after 30 minutes of ET1 or VEGFsp-treatment compared with non-treated transfectant-matched controls respectively. The % CFC=[Treated^{Ave}−Control^{Ave}]/Control^{Ave}×100. Although % CFC>25% is suggested as a significant difference, only values exhibiting >50% CFC and with % error range between duplicates less than 20% for both test and control samples were presented. The % error range=[Duplicateⁿ−Average]/Average. A % error >20% was accepted if the % CFC remained >50% using the lesser of the duplicates in calculating % CFC.

Statistical analysis. One way analysis of variance (ANOVA) followed by all pairwise multiple comparison Tukey test were performed after ascertaining normality using SigmaStat 2.03 software package. A P <0.05 was considered statistically significant.

### Results

DEspR expression is increased in human tumor cells and tumor vessels. DEspR-specific expression patterns were investigated in human cancer tissues and cells. Tumor tissue array analysis was performed using a human-specific anti-DEspR polyclonal-antibody [17]. Concordant with rat irradiation-induced mammary tumor model observations of rat-specific anti-DEspR antibody [13] immunostaining, immunohistochemical analysis of DEspR expression in human tumor tissue arrays detected increased DEspR expression in thin-walled tumor vascular endothelium in hepatic, pancreatic, stomach, breast, colon and lung cancer, compared with vascular endothelium in normal tissue biopsy cores respectively be it arterial or microvascular endothelium. Notably, vascular endothelium in stomach cancer metastatic foci in the lung and breast cancer metastatic foci in lymph node also exhibit increased DEspR immunostaining. Moreover, pancreatic, stomach, breast, lung, and colon tumor cells exhibit increased DEspR expression with sub-cellular localization in the cell membrane, cytoplasm and nuclear membrane. This increased DEspR expression in tumor neovessels and tumor cells demonstrated herein indicate that that DEspR plays a role in both tumor neovascularization and in tumorigenesis.

To further confirm expression in tumor cells DEspR-immunostaining of cancer cell-array testing different types of previously characterized, established cancer cell lines was next performed (Table 3). In contrast to a few cell lines tested with minimal if any DEspR expression, several cancer cell lines exhibit DEspR expression with nuclear membrane DEspR expression associated with high-nuclear grade (Table 3). Representative photomicrographs demonstrate tumor cell expression with strongest DEspR-immunostaining in nuclear membranes of most tumor cells, but not all. The selective nuclear membrane immunostaining confirms specificity of DEspR immunostaining, along with negative immunostaining of some cancer cell lines (Table 3). Importantly, these observations are concordant with the observations in cancer tissue sections described herein. Nuclear membrane localization indicates that DEspR can play a role in crosstalk between the cell membrane and nuclear membrane, beyond receptor-mediated signal transduction.

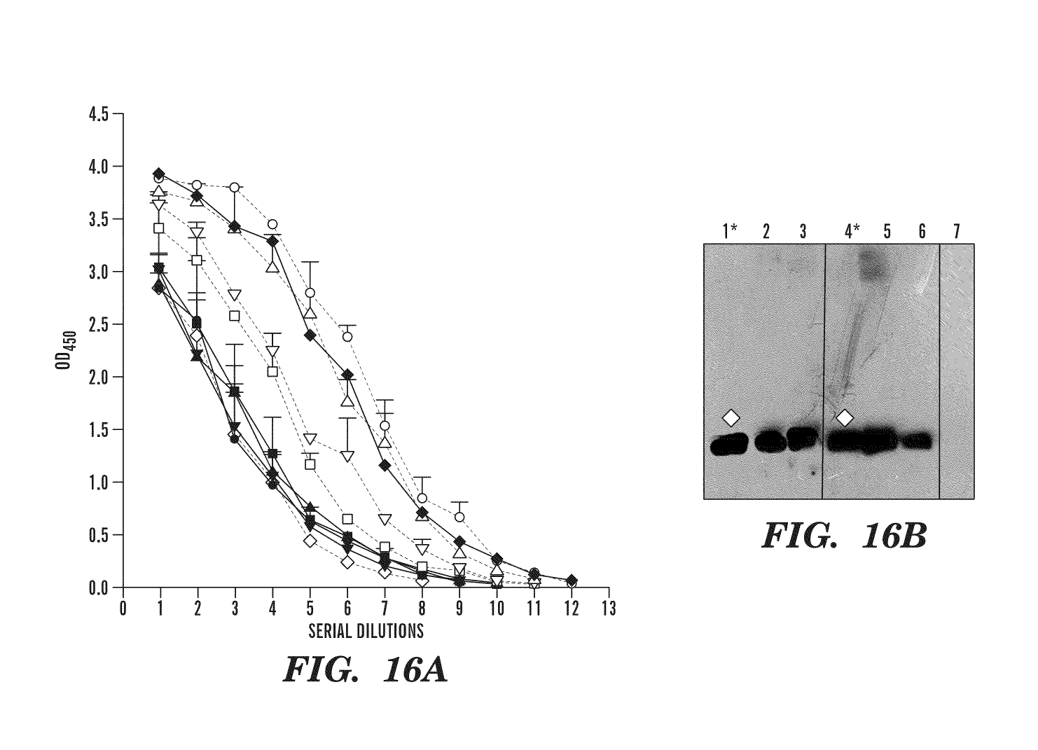

High-affinity anti-hDEspR monoclonal antibody generated against N-terminal 9-aa extra-cellular domain. In order to investigate anti-DEspR inhibition as an anti-angiogenic strategy, a human-specific anti-DEspR monoclonal antibody was developed using a 9-aa peptide spanning the N-terminal extracellular domain of human DEspR identical to the strategy use to develop the human-specific anti-DEspR polyclonal antibody used in DEspR immunostaining [17]. From 67 hybridoma clones, a preliminary screen identified top ten candidate monoclonal antibody hybridoma clones which were then analyzed for affinity to the 9-aa peptide N-terminal domain by indirect ELISA (FIG. 16A). Analysis of specificity by Western blot analysis of mab-mediated binding to hDEspR protein (10 kDa) isolated from Cos1-hDEspR transfectants in contrast to control non-transfected Cos1 cells identified hybridoma clone 7C5B2. As shown in FIG. 16B, 7C5B2 anti-hDEspR monoclonal antibody hybridoma clone exhibited specificity as both “super clone” supernatant and purified monoclonal antibody. Isotyping of 7C5B2 showed that this monoclonal antibody belongs to the murine IgG2b isotype class of antibodies.

Co-localization of DEspR and its ligand, VEGFsp in human umbilical vascular endothelial cells (HUVECs). Analysis of receptor-ligand co-localization by double immunostaining in HUVECs showed specific detection of DEspR on endothelial cell membrane cultured in pro-angiogenesis conditions using the anti-hDEspR monoclonal antibody. Double immunostaining detected co-localization of DEspR with its ligand VEGFsp using an anti-VEGFsp polyclonal antibody, thus demonstrating that anti-hDEspR monoclonal antibody specifically targets DEspR. Anti-DEspR polyclonal antibody also gave identical results.

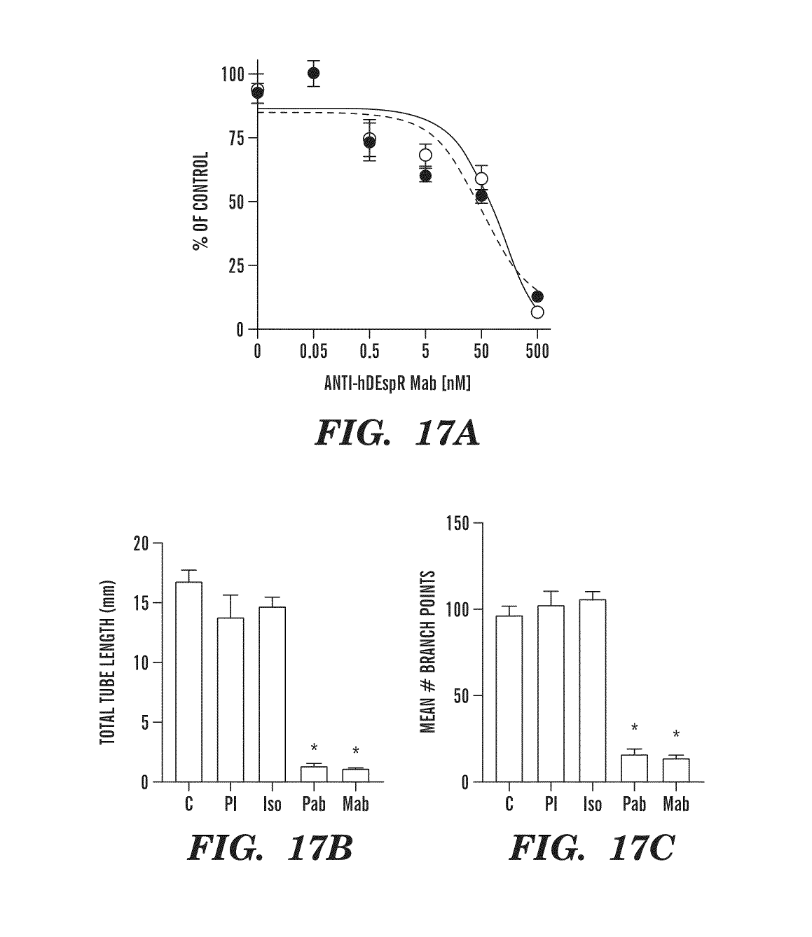

Anti-DEspR inhibition by anti-hDEspR polyclonal antibody and7C5B2 monoclonal antibody decrease angiogenesis. The effects of 7C5B2 monoclonal antibody inhibition of DEspR on angiogenesis using established in vitro HUVECs-based angiogenesis assays was then assessed. It was first showed that 7C5B2 monoclonal antibody detects cell-membrane DEspR expression in tubes/“neovessels” formed by HUVECs in pro-angiogenesis conditions, thus validating the use of this angiogenesis assay system. Next, two established parameters of in vitro angiogenesis were analyzed, total tube length and branching of neovessel-tubes formed by HUVECs in pro-angiogenesis conditions. Using varying doses of 7C5B2 monoclonal antibody from 0.05 to 500 nM, concentration dependence of angiogenesis inhibition is demonstrated for both total tube length and number of branch-points, and identifies 500 nM 7C5B2 monoclonal antibody as the full-strength inhibitory dose (FIG. 17A). This dose was then applied to repeat independent inhibition experiments comparing the newly developed 7C5B2 monoclonal antibody with the previously characterized anti-hDEspR polyclonal antibody. Compared to non-treated controls, and pre-immune and IgG2b-isotype-specific negative controls for polyclonal antibody and 7C5B2 monoclonal antibody respectively, 500 nM anti-hDEspR antibody inhibited angiogenesis, measured as total tube length and mean number of branch points, significantly (ANOVA with all pairwise multiple comparison Tukey test, P <0.01). Other angiogenesis parameters, number of tubes and branch-interconnections were also significantly inhibited. Concordantly, a polyclonal anti-VEGFsp antibody also inhibited angiogenesis in HUVECs.

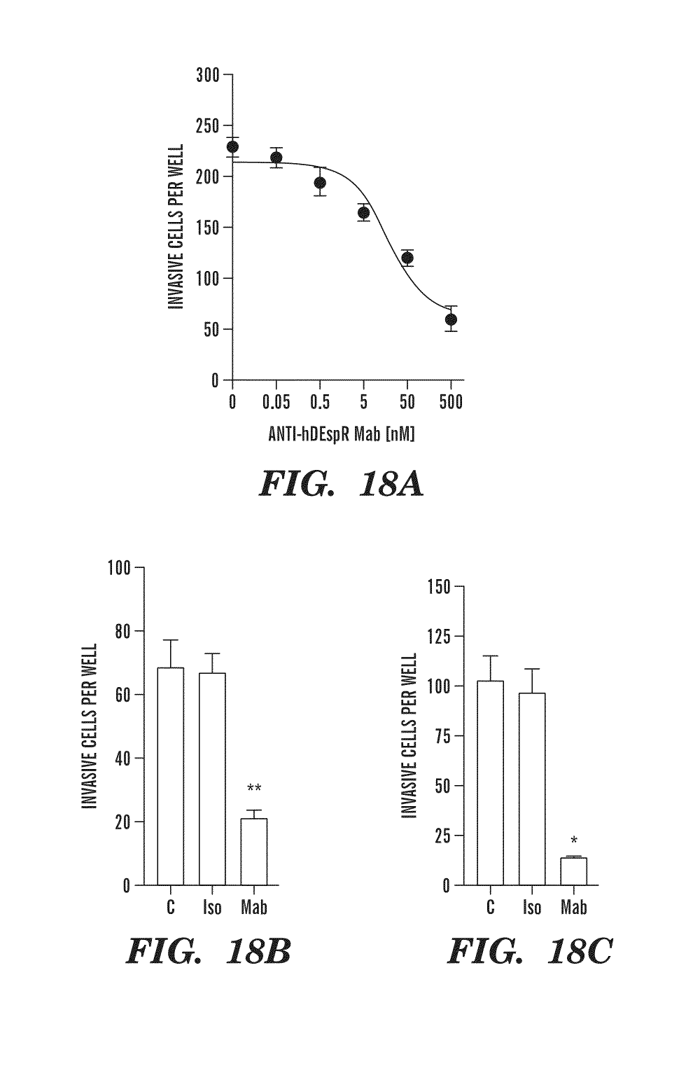

Analysis of anti-hDEspR 7C5B2 monoclonal antibody immunostaining and inhibition of tumor cell invasiveness. Having shown that DEspR inhibition reduces angiogenesis, the efficacy of 7C5B2 monoclonal antibody-mediated anti-DEspR inhibition on tumor cell invasiveness was next assessed since DEspR is detected in different tumor cell lines and cancer tissues. Two cancer cell lines representing aggressive triple negative breast cancer (TNBC) and pancreatic cancer, MDA-MB-468 and PANC-1 cancer cell lines respectively, were examined. Immunostaining with 7C5B2 monoclonal antibody detected nuclear- and cell-membrane DEspR expression in both cell lines, as well as cytoplasmic expression. Functional analysis detected concentration dependent inhibition of tumor cell invasiveness from 0.05 to 500 nM 7C5B2 monoclonal antibody, with an EC50 of 3.55±0.32 nM. Using 500 nM 7C5B2 monoclonal antibody, DEspR inhibition was observed in both MDA-MB-468 (FIG. 18B) and PANC1 (FIG. 18C) cells, compared to control non-treated cells and IgG2b-isotype treated cells respectively (ANOVA followed by all pairwise multiple comparison test, P <0.001 and P <0.01 respectively). These observations indicate dual effects of DEspR inhibition on both angiogenesis (FIG. 17B-17C) and tumor cell invasiveness (FIG. 18B-18C).

Anti-hDEspR 7C5B2 monoclonal antibody-immunostaining of tumor vascular endothelium and tumor cells. Having shown efficacy of DEspR-inhibition on angiogenesis and tumor cell invasiveness, 7C5B2 monoclonal antibody-immunostaining in breast and pancreatic cancer tissues in contrast to normal was next evaluated to confirm increased DEspR expression in tumor vascular endothelium and tumor cells as detected using anti-hDEspR polyclonal antibody, as well as to delineate DEspR-targeting profile of 7C5B2 monoclonal antibody.

Double immunostaining of DEspR and alpha smooth muscle actin (αSMA), to track microvascular pericytes and cancer tissue stromal myofibroblasts, detected minimal DEspR expression in normal breast tissue blood vessels and mammary epithelial cells, and normal αSMA expression in mammary myoepithelial cells and arteriolar smooth muscle cells highlighting minimal to no DEspR expression. In contrast, in a representative breast cancer tissue sections of ductal invasive carcinoma, double immunostaining detected prominent DEspR expression in tumor microvascular endothelium, in microvessels and arterioles co-expressing αSMA, as well as in ductal carcinoma epithelial cells. Increased tumor vascularization is also noted compared to non-cancer ‘normal’ control tissue.

Similarly, in normal pancreas, minimal DEspR expression is detected in microvessels, and in arterial endothelium in contrast to strong αSMA expression in arterial media smooth muscle cells. In contrast, DEspR expression is increased in pancreatic cancer αSMA-negative microvascular and αSMA-positive microvascular and arteriolar endothelium. As observed in breast cancer epithelial cells and in PANC-1 cancer cell line, pancreatic cancer ductal carcinoma epithelial cells exhibit marked DEspR-positive immunostaining.

Phosphoproteome analysis of DEspR signal transduction. Using a phosphoprotein-specific antibody-array, ligand-specific signal transduction pathways activated by DEspR upon binding to its dual ligands, ET1 and VEGFsp respectively in permanent Cos1-cell DEspR transfectants were identified (Table 4). Cos1 cells were used as these cells do not have endogenous DEspR, ET1a, or ET1b expression. Non-treated and treated Cos1-DEspR transfectants were compared. As shown in Table 4, regardless of ligand, DEspR's phosphoproteome (limited to signaling phosphoproteins with >50% CFC) activates signaling pathways known to be involved in mechanisms of angiogenesis, tumor cell invasiveness or metastasis. Additionally, some DEspR-phosphorylated signaling molecules for either ET1 or VEGFsp-activation of DEspR have been directly linked to either neuronal or hematopoietic stem cells, with some also implicated in cancer stem cell renewal such as ERK1/2, FAK, Met, PKC-alpha, SHP2, Smad, STAT1, and STAT3 (Table 3). It is noted herein that DEspR's phosphoproteome overlaps with VEGFR2/VEGF for some signaling molecules like FAK, ERK1/2, Raf, PKCa [19]. However, the collective signaling complexes of DEspR/ET1 and DEspR/VEGFsp (Table 3) are quite distinct from that described for VEGFR2/VEGFa [19], thus confirming non-redundant angiogenesis roles as deduced from null mutant abnormal angiogenesis phenotypes for DEspR [13] and VEGF [20,21] with identical embryonic lethality between embryonic E10.5 and E12.5 days, although VEGFR2 or Flk1 null mutants died earlier between E8.5-E9.5 days [22].

### Discussion

DEspR as a novel target for anti-tumor vascularization therapy. The detection of increased DEspR expression in tumor vascular endothelium, in contrast to normal tissue-matched controls, detection of DEspR expression in both αSMA-negative capillaries/microvessels and αSMA-positive arterioles and arteries in the tumor stroma, and successful inhibition of angiogenesis through DEspR-inhibition all demonstrate that DEspR is a novel target for therapies aimed at both tumor angiogenesis and at existing or ‘mature’ tumor microvasculature. More specifically, targeting DEspR on αSMA-positive microvessels can address anti-VEGF therapy-resistant tumors which are thought, without wishing to be limited or bound by a theory, to have a stromal vasculature no longer dependent on VEGF due their ‘maturation’ as marked by αSMA-positive pericyte sheath or non-dependent on VEGF due to “cooption of existing” microvasculature [2]. Furthermore, combined targeting DEspR along with anti-VEGF therapies can address the expected concomitant 10-fold increase in VEGFsp that accompanies the observed 10-fold increase in VEGF upon anti-VEGF therapy [10], since VEGF and VEGFsp originate from a common propeptide.

Insights from the ligand-specific DEspR phosphoproteome. Given that hypoxia inducible factor-1 alpha (HIF1α) stabilization induces VEGF, and hence VEGFsp, in hypoxia, phosphorylation of BRCA1 and induction of PCNA expression by VEGFsp-DEspR activation (Table 3), indicates that DEspR can contribute to the needed DNA repair response activated in hypoxia [24], thus allowing DEspR-positive endothelial and cancer cells to proliferate despite the hypoxic microenvironment, rather than undergo hypoxia-induced cell cycle arrest and apoptosis [24,25]. The hepatocyte growth factor receptor, MET, is induced upon ET1/DEspR stimulation and Smad1/5/9 is phosphorylated upon DEspR/VEGFsp activation, thus indicating a mechanism for crosstalk and/or redundancy among VEGFsp/DEspR, MET/HGF, and TGFβ/Smad pathways pertinent to angiogenesis in endothelial cells and invasiveness in cancer cells. Importantly, DEspR phosphorylates BRCA1 and STAT3 both of which have been shown to stabilize HIF1α, and along with Raf1, lead to the induction of VEGF, and hence VEGFsp. Furthermore, the phosphorylation of BRCA1 [26] by VEGFsp/DEspR and STAT3 by both ET1/DEspR and VEGFsp/DEspR, can both lead to DEspR-mediated stabilization of HIF1-alpha without the need for hypoxia, leading to constitutive HIF1-α mediated pro-angiogenic and pro-DNA repair response which can contribute to tumor resistance to conventional therapy.

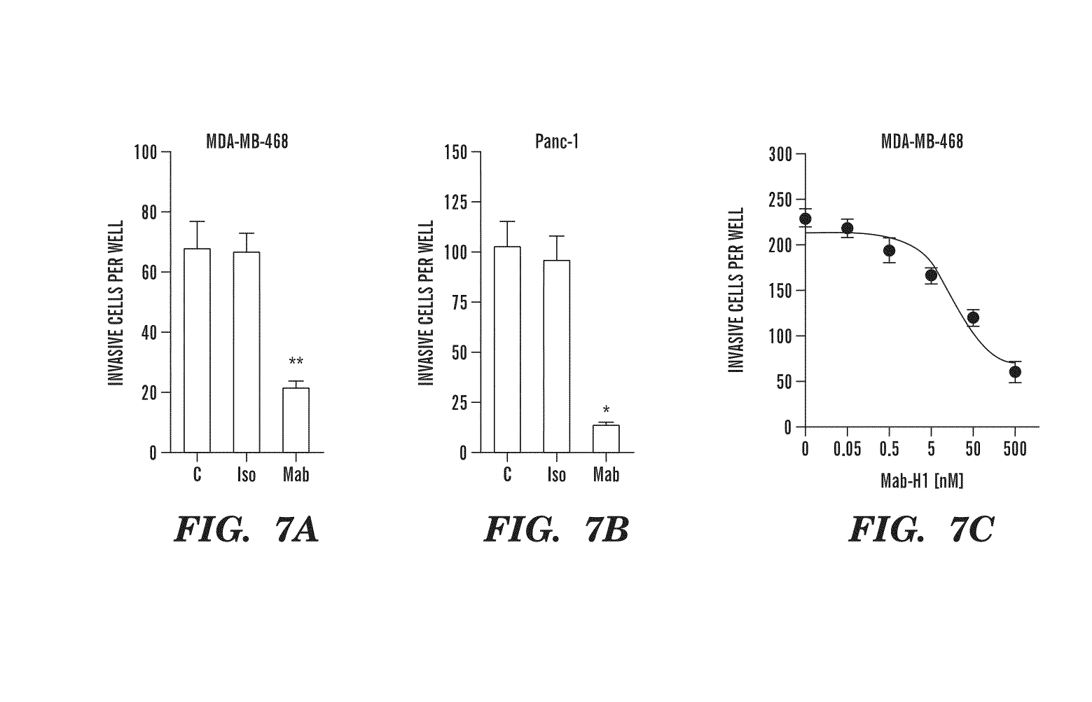

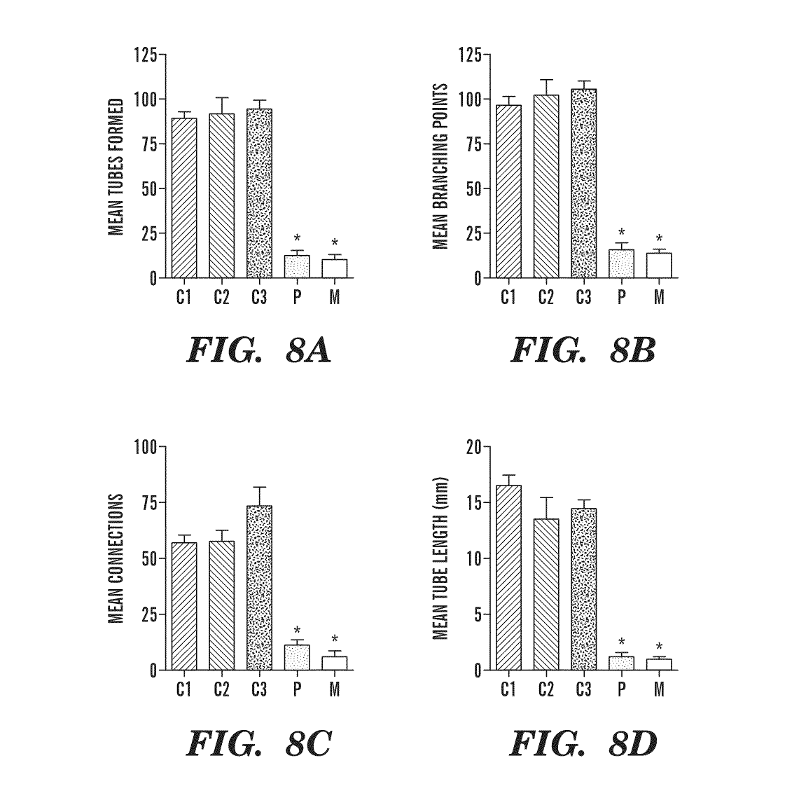

DEspR inhibition as target for dual anti-angiogenesis/anti-cancer cell invasiveness treatment paradigm. In addition to expression on tumor vascular endothelium, DEspR is expressed in solid tumor epithelial cells seen in both established cancer cell lines and histology sections of breast, pancreatic, lung, stomach, bladder and colon cancers. Just as anti-DEspR inhibition reduces in vitro angiogenesis (FIGS. 16A-16B), 7C5B2 monoclonal antibody-inhibition decreases tumor cell invasiveness in two aggressive cancer cell lines, breast cancer cell line MDA-MB-1-468) and pancreatic cancer cell line PANC-1 (FIGS. 17A-17C). Thus, targeting DEspR as a receptor involved in both angiogenesis and tumor cell invasiveness via anti-hDEspR monoclonal antibody-inhibition using the compositions and methods described herein provides a robust new anti-tumor therapy, and demonstrates the use of the anti-hDEspR 7C5B2 monoclonal antibody described herein aas an anti-hDEspR monoclonal antibody-therapeutic precursor.

Furthermore, dual-targeting of angiogenesis and metastasis mechanisms comprise novel methods for next-generation anti-cancer treatment strategies [2]. The data described herein demonstrate that targeting DEspR is can be used to achieve a dual-treatment paradigm. The increased expression in both pancreatic tumor neovessel and tumor cells, along with the inhibition of angiogenesis and pancreatic cancer cell line PANC-1 cell-invasiveness by anti-DEspR inhibition altogether indicate that anti-DEspR therapy can provide a new treatment approach for pancreatic cancer. The combinatorial anti-angiogenesis and anti-invasiveness caused by DEspR-inhibition, as shown herein, as well as targeting DEspR for dual tumor endothelial and tumor cell targeted-delivery, can be used, in some embodiments, as a therapeutic basis for next generation dual anti-tumor/anti-angiogenesis cancer therapies and methods thereof [2].

TABLE 3
Tumor array analysis of DEspR expression in different
cancers and cancer cell lines.

| | ↑tumor | | |
|---|---|---|---|
| Cancer tissue- | vascular | | Cancer cell lines |
| type | endothelium vs | Representative | DEspR-positive |
| (n) | normal | cancer types | DEspR-negative |
| Bladder (23) | 17/23 | Adenocarcinoma | *253J BV |
| | (74%) | Squamous cell ca | |
| | | Transitional cell ca | |
| Breast (36) | 34/36 | Invasive ductal ca | *MDA-MB-231 |
| | (94%) | Adenoca | *MDA-MB-468 |
| | | Medullary ca | |
| | | Invasive lobular ca | |
| Colon (6) | 5/6 | Adenoca | *SW480 |
| Liver (35) | 24/35 | Hepatocellular ca | HEP3B |
| | (68%) | Clear cell ca | HEPG2 |
| | | Bile duct ca | |
| Lung (2) | 2/2 | Adenocarcinoma | *NCI-H627 |
| | | | NCI-H292 |
| Pancreas (6) | 6/6 | Ductal carcinoma | *PANC-1 |
| Stomach (2) | Primary | Adenocarcinoma | na |
| | and in | | |
| | metastasis | | |
| | to lung | | |

*nuclear membrane immunostaining;
ca, carcinoma; cancer cell line nomenclature based on ATCC;
na, not available on cell-line array,
n, number of biopsy cores on tissue array.

TABLE 4
Phosphoproteome of hDEspR upon ET1 and VEGFsp stimulation respectively.

| | | | ET1 | VEGFsp | Pro- | Pro- | Pro- |
|---|---|---|---|---|---|---|---|
| Protein Name | Symbol | P*-Site | (% CFC) | (% CFC) | Angiogenesis | Cancer | Stem cell |
| Breast cancer type 1 | BRCA1 | S1497 | 32 | 82 | [26] | [27] | |
| susceptibility protein | | | | | | | |
| Cyclin-dependent protein- | CDK1/2 | T14/Y15 | 53 | −16 | | [28] | |
| serine kinase ½ | | Y15 | 281 | −57 | | | |
| Extracellular regulated | ERK1/2 | T202 + Y204; | 135 | −25 | [29, 30] | [31-33] | NSC: [34] |
| protein-serine kinase ½ | | T185 + Y187 | | | | | CSC: [35] |
| (p44/p42 MAP kinases) | | | | | | | |
| Focal adhesion protein- | FAK | S722 | 55 | −38 | [36, 37] | Metastasis: | NSC: [34] |
| tyrosine kinase | | S732 | 62 | −11 | | [37, 38] | CSC: [37] |
| | | Panspecific | 205 | 0 | | | |
| Hepatocyte growth factor | Met | Panspecific | 384 | 0 | [39-41] | Metastasis: | [43] |
| receptor-tyrosine kinase | | | | | | [42]; | |
| | | | | | | Resistance: | |
| | | | | | | [39] | |
| Proliferating cell nuclear | PCNA | Panspecific | −47 | 119 | | [44] | |
| antigen | | | | | | | |
| Protein-serine kinase C- | PKCa | T638/T641 | 137 | −17 | [45, 46] | | |
| alpha | | | | | | | |
| Protein-serine kinase C- | PKCe | Panspecific | 103 | −29 | [47-50] | [50] | NSC: |
| epsilon | | | | | | | [34]; |
| | | | | | | | CSC: [50] |
| Raf1 proto-oncogene- | Raf1 | S259 | 12 | 63 | [51] | [52] | |
| encoded protein-serine | | | | | | | |
| kinase | | | | | | | |
| SH2 domain-containing | Shc1 | Y349, | 9 | 97 | [53-55] | [53, 56, 57] | |
| transforming protein 1 | or | Y350 | | | | | |
| | ShcA | | | | | | |
| Protein-tyrosine | SHP2 | S576 | 14 | 97 | [58-60] | [58, 61, 62] | [61, 63-65] |
| phosphatase 1D | | | | | | | |
| SMA- and mothers against | Smad | S463 + S465/ | 18 | 147 | [30] | [66] | HSC: [67] |
| decapentaplegic homologs | 1/5/9 | S465 + S467 | | | | | |
| 1/5/9 | | | | | | | |
| Src proto-oncogene- | Src | Y529 | −20 | 73 | [47] | [68-70] | |
| encoded protein-tyrosine | | Y418 | −11 | 174 | | | |
| kinase | | | | | | | |
| Signal transducer and | STAT1 | S727 | 86 | 123 | | Metastasis, | CSC: [72] |
| activator of transcription 1 | | Y701 | 95 | 557 | | invasiveness: | |
| | | | | | | [71] | |
| Signal transducer and | STAT3 | S727 | 133 | 126 | [73-75] | [74] | NSC: [76] |
| activator of transcription 3 | | | | | | Invasiveness: | |
| | | | | | | [75] | |

CSC, cancer stem cell;
ET1, endothelin 1;
hDEspR, human dual endothelin-1/vascular endothelial growth factor-signal peptide receptor;
NSC, neural stem cell;
VEGFsp, vascular endothelial growth factor-signal peptide;
% CFC, percentage change in treated vs non-treated control averages: % CFC = [Treated − Control]/Control ave] × 100.
Phospho-site, phosphorylation site detected with phosphorylated site-specific antibodies.
Data represent >50% CFC taken from mean of treated vs control non-treated duplicates (A, B) with % error range <20%.
% error range = [Treated_{A} − ave]/ave × 100.
Kinexus antibody array: phosphoprotein-specific ab to detect phosphorylation changes, and panspecific antibodies to detect expression changes.

[1] D. Hanahan, R. A. Weinberg, The hallmarks of cancer, Cell 100 (2000) 57-70.
[2] G. Bergers, D. Hanahan, Modes of resistance to anti-angiogenic therapy, Nature Reviews—Cancer 8 (2008) 592.
[3] A. Abdollahi, J. Folkman, Evading tumor evasion: current concepts and perspectives of anti-angiogenic cancer therapy, Drug Resist. Updat. 13 (2010) 16-28.
[4] N. Ferrara, Pathways mediating VEGF-independent tumor angiogenesis, Cytokine Growth Factor Rev. 21 (2010) 21-26.
[5] S. Loges, T. Schmidt, P. Carmeliet, Mechanisms of resistance to anti-angiogenic therapy and development of third-generation anti-angiogenic drug candidates, Genes & Cancer 1 (2010) 12-25.
[6] P. Carmeliet, Angiogenesis in life, disease and medicine, Nature 438 (2005) 932-936.
[7] Y. Crawford, N. Ferrara, Mouse models to investigate anti-cancer effects of VEGF inhibitors, Methods Enzymol. 445 (2008) 125-139.
[8] J. M. Ebos, C. R. Lee, W. Cruz-Munoz, G. A. Bjarnason, J. G. Christensen, R. S. Kerbel, Accelerated metastasis after short-term treatment with a potent inhibitor of tumor angiogenesis, Cancer Cell 15 (2009) 232-239.
[9] M. Paez-Ribes, E. Allen, J. Hudock, T. Takeda, H. Okuyama, F. Vinals, M. Inoue, G. Bergers, D. Hanahan, O. Casanovas, Antiangiogenic therapy elicits malignant progression of tumors to increased local invasion and distant metastasis, Cancer Cell 15 (2009) 220-231.
[10] C. G. Willett, Y. Boucher, D. G. Duda, E. diTomaso, L. L. Munn, R. T. Tong, S. V. Kozin, L. Petit, R. K. Jain, D. C. Chung, D. V. Sahani, S. P. Kalva, K. S. Cohen, D. T. Scadden, A. J. Fischman, J. W. Clark, D. P. Ryan, A. X. Zhu, L. S. Blaszkowsky, P. C. Shellito, M. Mino-Kenudson, G. Y. Lauwers, Surrogate markers for antiangiogenic therapy and dose-limiting toxicities for Bevacizumab with radiation and chemotherapy: continued experience of a phase I trial in rectal cancer patients, J. Clin. Oncol. 23 (2005) 8136-8139.
[11] G. Bocci, S. Man, S. K. Green, G. Francia, J. M. Ebos, J. M. du Manoir, A. Weinerman, U. Emmenegger, L. Ma, P. Thorpe, A. Davidoff, J. Huber, D. J. Hicklin, R. S. Kerbel, Increased plasma VEGF as a surrogate marker for optimal therapeutic dosing of VEGF receptor-2 monoclonal antibodies, Cancer Res. 64 (2004) 6616-6625.
[12] N. Ruiz-Opazo, K. Hirayama, K. Akimoto, V. L. M. Herrera, Molecular characterization of a dual Endothelin-1/Angiotensin II Receptor, Molecular Medicine 4 (1998) 96-108.
[13] V. L. M. Herrera, L. R. B. Ponce, P. D. Bagamasbad, B. D. VanPelt, T. Didishvili, N. Ruiz-Opazo, Embryonic lethality in Dear gene-deficient mice: new player in angiogenesis, Physiol. Genomics 23 (2005) 257-268.
[14] K. Hosoda, R. E. Hammer, J. A. Richardson, A. G. Baynash, J. C. Cheung, A. Giaid, M. Yanagisawa, Targeted and natural (piebald-lethal) mutations of endothelin-B receptor gene produce megacolon associated with spotted coat color in mice, Cell 79 (1994) 1267-1276.
[15] D. E. Clouthier, K. Hosoda, J. A. Richardson, S. C. Williams, H. Yanagisawa, T. Kuwaki, M. Kumada, R. E. Hammer, M. Yanagisawa, Cranial and cardiac neural crest defects in endothelin-A receptor-deficient mice, Development 125 (1998) 813-824.
[16] A. Bagnato, L. Rosano, The endothelin axis in cancer, Int. J. Biochem. Cell Biol. 40 (2008) 1443-1451.
[17] N. Glorioso, V. L. M. Herrera, P. Bagamasbad, F. Filigheddu, C. Troffa, G. Argiolas, E. Bulla, J. L. Decano, N. Ruiz-Opazo, Association of ATPJAJ and Dear SNP-haplotypes with essential hypertension: sex-specific and haplotype-specific effects, Circ. Res. 100 (2007) 1522-1529.
[18] Y. Matsuo, M. Raimondo, T. A. Woodward, M. B. Wallace, K. R. Gill, Z. Tong, M. D. Burdick, Z. Yang, R. M. Strieter, R. M. Hoffman, S. Guha, CXC-chemokine/CXCR2 biological axis promotes angiogenesis in vitro and in vivo in pancreatic cancer, Int. J. Cancer 125 (2009) 1027-1037.
[19] A. K. Ollson, A. Dimberg, J. Kreuger, L. Claesson-Welsh, VEGF receptor signaling—in control of vascular function. Nat Reviews: Mol. Cell. Biol. 7 (2006) 359-371.
[20] P. Carmeliet, V. Ferreira, G. Breier, S. Pollefeyt, L. Lieckens, M. Gertsenstein, M. Fahrig, A. Vandenhoeck, H. Kendraprasad, C. Eberhardt, C. Declercq, J. Pawling, L. Moons, D. Collen, W. Risau, A. Nagy, Abnormal blood vessel development and lethality in embryos lacking a single VEGF allele, Nature 380 (1996) 435-439.
[21] N. Ferrara, K. Carver-Moore, H. Chen, M. Dowd, L. Lu, K. S. O'Shea, L. Powell-Braxton, K. J. Hillan, M. W. Moore, Heterozygous embryonic lethality induced by targeted inactivation of the VEGF gene, Nature 380 (1996) 439-442.
[22] F. Shalaby, J. Rossant, T. P. Yamaguchi, M. Gertsenstein, X. F. Wu, M. L. Bretman, A. C. Schuh, Nature 376 (1995) 62-66.
[23] M. Hidalgo, Pancreatic Cancer, New Engl. J. Med. 362 (2010) 1605-1617.
[24] M. L. Coleman, P. J. Ratcliffe, Angiogenesis: escape from hypoxia, Nat. Med. 15 (2009) 491-492.
[25] E. M. Hammond, A. J. Biaccia, The role of ATM and ATR in the cellular response to hypoxia and re-oxygenation, DNA Repair 3 (2004) 117-1122.
[26] H. J. Kang, H. J. Kim, J. K. Rih, T. L. Mattson, K. W. Kim, C. H. Cho, J. S. Isaacs, I. Bae, BRCA1 plays a role in the hypoxic response by regulating HIF-1a stability and by modulating vascular endothelial growth factor expression, J. Biol. Chem. 281 (2006) 13047-13056.
[27] C. Hesling, M. D'Incan, C. D'Incan, P. Souteyrand, J. C. Monboisse, S. Pasco, J. C. Madelmont, Y. J. Bignon, Downregulation of BRCA1 in A375 melanoma cell line increases radio-sensitivity and modifies metastatic and angiogenic gene expression, J. Invest. Dermatol. 122 (2004) 369-380.
[28] N. Johnson, D. Cai, R. D. Kennedy, S. Pathania, M. Arora, Y. C. L1, A. D. D′ Andrea, J. D. Parvin, G. I. Shapiro, Cdk1 participates in BRCA1-dependent S phase checkpoint control in response to DNA damage, Mol. Cell. 35 (2009) 327-339.
[29] J. Xu, X. Liu, Y. Jiang, L. Chu, H. Hao, Z. Liu, C. Verfullie, J. Zweier, K. Gupta, Z. Liu, MAPK/ERK signaling mediates VEGF-induced bone marrow stem cell differentiation into endothelial cell, J. Cell. Mol. Med. 12 (2008) 2395-2406.
[30] E. M. Langenfeld, Y. Kong, J. Langenfeld, Bone morphogenetic protein-2-induced transformation involves the activation of mammalian target of rapamycin, Mol. Cancer. Res. 3 (2005) 679-684.
[31] J. A. Gollob, S. Wilhelm, C. Carter, S. L. Kelley, Role of Raf kinase in cancer: therapeutic potential of targeting the Raf/MEK/ERK signal transduction pathway, Semin. Oncol. 33 (2006) 392-406.
[32] K. Balmano, S. J. Cook, Tumor cell survival signaling by the ERK1/2 pathway, Cell Death Differ. 16 (2009) 368-377.
[33] C. Fremin, S. Meloche, From basic research to clinical development of MEK1/2 inhibitors for cancer therapy, J. Hematol. Oncology 3 (2010) 8-18.
[34] R. Morishita, H. Ueda, H. Ito, J. Takasaki, K. Nagata, T. Asano, Involvement of Gq/11 in both integrin signal-dependent and -independent pathways regulating endothelin-induced neural progenitor proliferation, Neurosci. Res. 59 (2007) 205-214.
[35] Y. Wang, Y. Zhu, F. Qiu, T. Zhang, Z. Chen, S. Zheng, J. Huang, Activation of Akt and MAPK pathways enhances the tumorigenicity of CD133+ primary colon cancer cells, Carcinogenesis 2010 Jun. 8. [Epub ahead of print]
[36] K. Vadali, X. Cai, M. D. Schaller, Focal adhesion kinase: an essential kinase in the regulation of cardiovascular functions, IUBMB Life 59 (2007) 709-716.
[37] M. Luo, J. L. Guan, Focal adhesion kinase: a prominent determinant in breast cancer initiation, progression and metastasis, Cancer Lett. 289 (2010) 127-139.
[38] P. P. Provenzano, D. R. Inman, K. W. Eliceiri, H. E. Beggs, P. J. Keely, Mammary epithelial-specific disruption of focal adhesion kinase retards tumor formation and metastasis in a transgenic mouse model of human breast cancer, Am. J. Pathol. 173 (2008) 1551-1565.
[39] S. Fan, Y. Xian, J. A. Wang, R. Q. Yuan, Q. Meng, Y. Cao, J. J. Laterra, I. D. Goldberg, E. M. Rosen, The cytokine hepatocyte growth factor/scatter factor inhibits apoptosis and enhances DNA repair by a common mechanism involving signaling through phosphatidyl inositol 3′ kinase, Oncogene 19 (2000) 2212-2223.
[40] E. S. Colombo, G. Menicucci, P. G. McGuire, A. Das, Hepatocyte growth factor/scatter factor promotes retinal angiogenesis through increased urokinase expression, Invest. Ophthalmol. Vis. Sci. 48 (2007) 1793-1800.
[41] K. Matsumoto, T. Nakamura, NK4 gene therapy targeting HGF-Met and angiogenesis, Front. Biosci. 13 (2008) 1943-1951.
[42] P. C. Ma, M. S. Tretiakova, V. Nallasura, R. Jagadeeswaran, A. N. Husain, R. Salgia, Downstream signaling and specific inhibition of c-MET/HGF pathway in small cell lung cancer: implications for tumor invasion, Br. J. Cancer 97 (2007) 368-377.
[43] Z. Yang, W. Wang, D. Ma, Y. Zhang, L. Wang, Y. Zhang, S. Xu, B. Chen, D. Miao, K. Cao, W. Ma, Recruitment of stem cells by hepatocyte growth factor via intracoronary gene transfection in the postinfarction heart failure, Sci. China C. Life Sci. 50 (2007) 748-752.
[44] R. Stuart-Harris, C. Caldas, S. E. Pinder, P. Pharoah, Proliferation markers and survival in early breast cancer: a systematic review and meta-analysis of 85 studies in 32,825 patients, Breast 17 (2008) 323-334.
[45] M. Wellner, C. Maasch, C. Kupprion, C. Lindschau, F. C. Luft, H. Haller, The proliferative effect of vascular endothelial growth factor requires protein kinase C-alpha and protein kinase C-zeta, Arterioscler. Thromb. Vasc. Biol. 19 (1999) 178-185.
[46] H. Xu, P. Czerwinski, M. Hortmann, H. Y. Sohn, U. Forstermann, H. L1, Protein kinase C alpha promotes angiogenic activity of human endothelial cells via induction of vascular endothelial growth factor, Cardiovasc. Res. 78 (2008) 349-355.
[47] G. E. Davis, W. Koh, A. N. Stratman, Mechanisms controlling human endothelial lumen formation and tube assembly in three-dimensional extracellular matrices, Birth Defects Research 81 (2007) 270-285.
[48] S. Yamamura, P. R. Nelson, K. C. Kent, Role of protein kinase C in attachment, spreading, and migration of human endothelial cells, J. Surg. Res. 63 (1996) 349-354.
[49] A. M. Gardner, M. E. Olah, Distinct protein kinase C isoforms mediate regulation of vascular endothelial growth factor expression by A2A adenosine receptor activation and phorbol esters in pheochromocytoma PC12 cells, J. Biol. Chem. 278 (2003) 15421-15428.
[50] M. C. Heidkamp, A. L. Bayer, B. T. Scully, D. M. Eble, A. M. Samarel, Activation of focal adhesion kinase by protein kinase C epsilon in neonatal rat ventricular myocytes, Am. J. Physiol. Heart Circ. Physiol. 285 (2003) H1684-H1696.
[51] M. Malecki, M. Seneta, J. Miloszewska, H. Trembacz, M. Przbyszewska, P. Janik, Role of v-Raf and truncated form RAF1 in the induction of vascular endothelial growth factor and vascularization, Oncol. Rep. 11 (2004) 161-165.
[52] F. J. Hoogwater, M. W. Nijkamp, N. Smakman, E. J. Steller, B. L. Emmink, B. F. Westendorp, D. A. Raats, M. R. Sprick, U. Schaefer, W. J. Van Houdt, M. T. De Bruijn, R. C. Schackmann, P. W. Derksen, J. P. Medema, H. Walczak, I. H. Borel Rinkes, O. Kranenberg, Oncogenic K-Ras turns death receptors into metastasis-promoting receptors in human and mouse colorectal cancer cells, Gastroenterology 138 (2010) 2357-2367.
[53] J. Ursini-Siegel, W. R. Hardy, D. Zuo, S. H. L. Lam, V. Sanguin-Gendreau, R. D. Cardiff, T. Pawson, W. Muller, ShcA signaling is essential for tumor progression in mouse models of human breast cancer, EMBO J. 27 (2008) 910-920.
[54] E. Audero, I. Cascone, F. Maniero, L. Napione, M. Arese, L. Lanfrancone, F. Bussolino, Adaptor ShcA protein binds tyrosine kinase Tie2 receptor and regulates migration and sprouting but not survival of endothelial cells, J. Biol. Chem. 279 (2004) 13224-13233.
[55] C. Saucier, H. Khoury, K. M. Lai, P. Peschard, D. Dankort, M. A. Naujokas, J. Holash, G. D. Yancopoulos, W. J. Muller, T. Pawson, M. Park, The Shc adaptor protein is critical for VEGF induction by Met/HGF and Erb B2 receptors and for early onset of tumor angiogenesis, Proc. Natl. Acad. Sci. 101 (2004) 2345-2350.
[56] J. J. Northey, J. Chmielecki, E. Ngan, C. Russo, M. G. Annis, W. J. Muller, P. M. Siegel, Signaling through ShcA is required for TGF-beta and Neu/ErbB-2 induced breast cancer cell motility and invasion, Mol. Cell. Biol. 28 (2008) 3162-3176.
[57] C. Saucier, V. Papavailiou, A. Palazzo, M. A. Naujokas, R. Kremer, M. Park, Use of signal specific receptor tyrosine kinase oncoproteins reveals that pathways downstream from Grb2 or Shc are sufficient for cell transformation and metastasis, Oncogene 21 (2002) 1800-1811.
[58] Y. M. Agazie, N. Movilla, I. Ischenko, M. J. Hayman, The phosphotyrosine phosphatase SHP2 is a critical mediator of transformation induced by the oncogenic fibroblast growth factor receptor 3, Oncogene 22 (2003) 6909-6918.
[59] R. D. Chemock, R. P. Cherla, R. K. Ganju, SHP2 and cbl participate in alpha-chemokine receptor CXCR4-mediated signaling pathways, Blood 97 (2001) 608-615.
[60] M. B. Marron, D. P. Hughes, M. J. McCarthy, E. R. Beaumont, N. P. Brindle, Tie-1 receptor tyrosine kinase endodomain interaction with SHP2: potential signaling mechanisms and roles in angiogenesis, Adv. Exp. Med. Biol. 476 (2000) 35-46.
[61] X. Zhou, J. Coad, B. Ducatman, Y. M. Agazie, SHP2 is up-regulated in breast cancer cells and in infiltrating ductal carcinoma of the breast, implying its involvement in breast oncogenesis, Histopathology 53 (2008) 389-402.
[62] X. Zhou, Y. M. Agazie, Molecular mechanism for SHP2 in promoting HER2-induced signaling and transformation, J. Biol. Chem. 284 (2009) 12226-12234.
[63] K. Hagihara, E. E. Zhang, Y. H. Ke, G. Liu, J. J. Liu, Y. Rao, G. S. Feng, Shp2 acts downstream of SDF-1 alpha/CXCR4 in guiding granule cell migration during cerebellar development, Dev. Biol. 334 (2009) 276-284.
[64] D. Wu, Y. Pang, Y. Ke, J. Yu, Z. He, L. Tautz, T. Mustelin, S. Ding, Z. Huang, G. S. Feng, A conserved mechanism for control of human and mouse embryonic stem cell pluripotency and differentiation by Shp2 tyrosine phosphatase, PLoS One 4 (2009) e4914.
[65] Y. Ke, E. E. Zhang, K. Hagihara, D. Wu, Y. Pang, R. Klein, T. Curran, B. Ranscht, G. S. Feng, Deletion of Shp2 in the brain leads to defective proliferation and differentiation in neural stem cells and early postnatal lethality, Mol. Cell. Biol. 27 (2007) 6706-6717.
[66] I. M. Liu, S. H. Schilling, K. A. Knouse, L. Choy, R. Derynck, X. F. Wang, TGFbeta-stimulated Smad1/5 phosphorylation requires the ALK5 L45 loop and mediates the pro-migratory TGFbeta switch, EMBO J. 28 (2009) 88-98.
[67] U. Blank, G. Karlsson, S. Karlsson, Signaling pathways governing stem-cell fate, Blood 111 (2008) 494-503.
[68] F. M. Johnson, G. E. Gallick, S R C family nonreceptor tyrosine kinases as molecular targets for cancer therapy, Anticancer Agents Med. Chem. 7 (2007) 651-659.
[69] E. H. Lin, A. Y. Hui, J. A. Meens, E. A. Tremblay, E. Schaefer, B. E. Elliott, Disruption of Ca2+-dependent cell-matrix adhesion enhances c-Src kinase activity, but causes dissociation of the c-Src/FAK complex and dephosphorylation of tyrosine-577 of FAK in carcinoma cells, Exp. Cell Res. 293 (2004) 1-13.
[70] B. Mezquita, J. Mezquita, M. Pau, C. Mezquita, A novel intracellular isoform of VEGFR-1 activates Src and promotes cell invasion in MDA-MB-231 breast cancer cells, J. Cell Biochem. 110 (2010) 732-742.
[71] J. Schultz, D. Koczan, U. Schmitz, S. M. Ibrahim, D. Pilch, J. Landsberg, M. Kunz, Tumor-promoting role of signal transducer and activator of transcription (Stat)1 in late-stage melanoma growth, Clin. Exp. Metastasis 27 (2010) 133-140.
[72] M. Heuser, R. K. Humphries, Biologic and experimental variation of measured cancer stem cells, Cell Cycle 9 (2010) 909-912.
[73] J. E. Jung, H. G. Lee, L H. Cho, D. H. Chung, S. H. Yoon, Y. M. Yang, J. W. Lee, S. Choi, J. W. Park, S. K. Ye, M. H. Chung, STAT3 is a potential modulator of HIF-1-mediated VEGF expression in human renal carcinoma cells, FASEB J. 19 (2005) 1296-1298.
[74] A. Jarnicki, T. Putoczki, M. Ernst, Stat3. linking inflammation to epithelial cancer—more than a “gut” feeling? Cell Div. 5 (2010) 14.
[75] H. Yu, D. Pardoll, R. Jove, STATs in cancer inflammation and immunity: a leading role for STAT3, Nat. Rev. Cancer 9 (2009) 798-809.
[76] M. V. Covey, S. W. Levison, Leukemia inhibitory factor participates in the expansion of neural stem/progenitors after perinatal hypoxia/ischemia, Neuroscience 148 (2007) 501-509.

### Example 4

### 7C5B2 Antibody Sequencing and Hdespr Composite Human Antibody Variant Generation

Described herein are sequencing results obtained from the monoclonal antibody expressed by the murine hybridoma 7C5B2 (anti-hDEspR), in which the heavy and light chain V-region (V_{H} and V_{L}) sequences of the 7C5B2 antibody have been determined and exemplary anti-hDEspR composite human antibody variants have been designed.

From viable frozen hybridoma cell pellets, RNA was extracted and PCR amplification of antibody specific transcripts was performed after reverse transcription of mRNA. The nucleotide and amino acid sequences of the antibody heavy and light chain V-regions were determined and the sequence data was analyzed. Fully humanized antibodies were then designed using Composite Human Antibody™ technology, as described herein.

### Methods and Results

### RNA Extraction, RT-PCR and Cloning

RNA was extracted from a cell pellet using an RNAqueous®-4PCR kit (Ambion cat. no. AM1914). RT-PCR was performed using degenerate primer pools for murine signal sequences with constant region primers for each of IgGVH, IgMVH, IgκVL and IgλVL. Heavy chain V-region RNA was amplified using a set of six degenerate primer pools (HA to HF) and light chain V-region mRNA was amplified using a set of eight degenerate primer pools, seven for the κ cluster (KA to KG) and one for the λ cluster (LA).

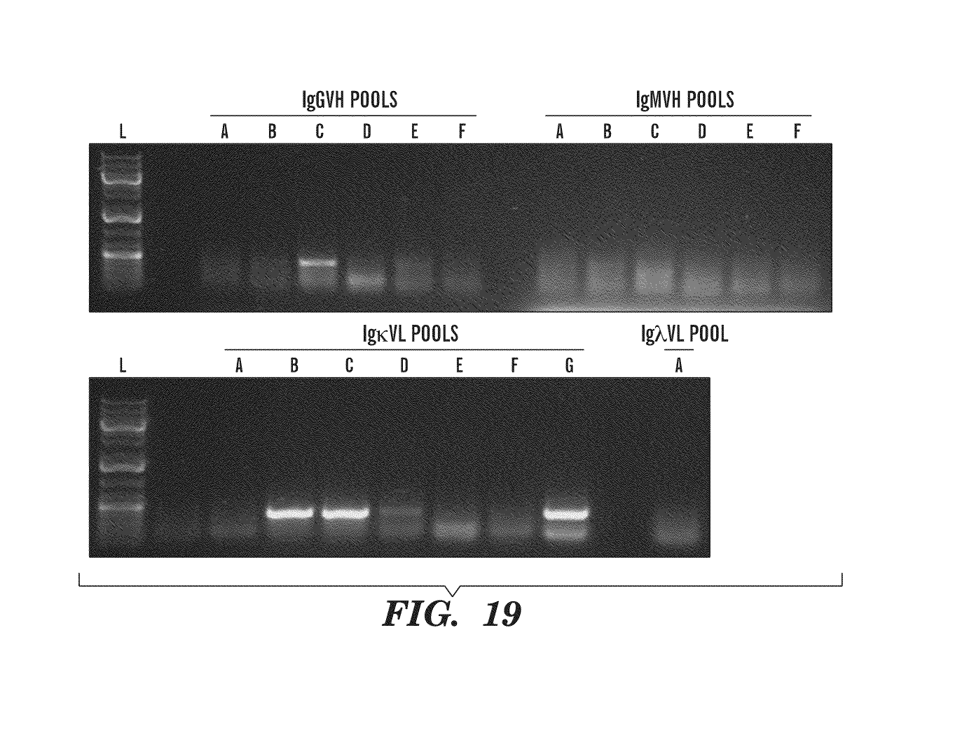

For the heavy chain V-region, amplification products of the expected size were obtained from the IgGVH reverse transcription primer and primer pool HC. For the light chain V-region, amplification products were obtained from the IgκVL reverse transcription primer and light chain primer pools KB, KC, KD, and KG (FIG. 19). The PCR products from each of the above pools were purified and cloned into a ‘TA’ cloning vector (pGEM (R)-T Easy, Promega cat. no. A1360). Six VH and 24 Vκ clones were sequenced.

A single functional VH gene was identified in five clones from pool HC and a single functional Vκ gene sequence was identified in six clones from primer pool KG. The twelve clones from primer pools KB and KC were found to contain an aberrant transcript (GenBank accession number M35669) normally associated with the hybridoma fusion partner SP2/0 and the six clones from pool KD were found to not contain a functional Vκ transcript.

### Chimeric Antibody

VH and Vκ (pool KG) genes were PCR amplified using primers that introduced flanking restriction enzyme sites for cloning into Antitope's VH and Vκ chain expression vectors. The VH region was cloned using MluI and HindIII sites, and the Vκs region were cloned using BssHII and BamHI restriction sites. All constructs were confirmed by sequencing.

The chimeric antibody constructs were transiently transfected into HEK293 cells using calcium phosphate precipitation. The transient transfections were incubated for three days prior to harvesting supernatants.

### Sequence Analysis

Analysis of sequences obtained from the hybridoma 7C5B2 is summarized in Table 1. The heavy and light chain V-regions show good homology to their closest human germline sequences (64% and 82%, respectively) and the individual framework sequences have close homologues in the human germline database.

### Design of Composite Human Antibodies

Design of COMPOSITE HUMAN ANTIBODY™ Variable Region Sequences

Structural models of the mouse anti-hDEspR 7C5B2antibody V regions were produced using Swiss PDB and analysed in order to identify important “constraining” amino acids in the V regions that were likely to be essential for the binding properties of the antibody. Residues contained within the CDRs (using Kabat definition) together with a number of framework residues were considered to be important. Both the VH and Vκ sequences of anti-hDEspR contain typical framework residues and the CDR 1, 2 and 3 motifs are comparable to many murine antibodies.

From the above analysis, it was considered that composite human sequences of anti-hDEspR could be created with a wide latitude of alternatives outside of CDRs but with only a narrow menu of possible alternative residues within the CDR sequences. Analysis indicated that corresponding sequence segments from several human antibodies could be combined to create CDRs similar or identical to those in the murine sequences. For regions outside of and flanking the CDRs, a wide selection of human sequence segments were identified as components of the novel Composite Human Antibody™ V regions described herein (see Table 1).

### Design of Variants

Based upon the above analysis, a large preliminary set of sequence segments that could be used to create anti-hDEspR COMPOSITE HUMAN ANTIBODY™ variants were selected and analysed using iTope™ technology for in silico analysis of peptide binding to human MHC class II alleles (Perry et al 2008), and using the TCED™ (T Cell Epitope Database) of known antibody sequence-related T cell epitopes (Bryson et al 2010). Sequence segments that were identified as significant non-human germline binders to human MHC class II or that scored significant hits against the TCED™ were discarded. This resulted in a reduced set of segments, and combinations of these were again analysed, as above, to ensure that the junctions between segments did not contain potential T cell epitopes.

Selected segments were then combined to produce heavy and light chain V region sequences for synthesis. For anti-hDEspR, five VH chains (SEQ ID NO: 13-SEQ ID NO: 17) and two Vκ chains (SEQ ID NO: 18 and SEQ ID NO: 19) were designed with sequences as detailed herein.

### Example 5

### Treatment at Onset of Acute Stroke in Spontaneously Stroke-Prone, Hypertensive-Hyperlipidemic Rat Model (spTg25 Rat Model)

spTg25 rats (Decano et al. Circulation. 2009 Mar. 24; 119(11):1501-9) are genetically hypertensive, and transgenic for the human cholesteryl ester transfer protein (CETP) thus exhibiting hypercholesterolemia and hypertriglyceridemia on normal rat chow, and become stroke prone on 0.4% NaCl normal rat chow. Onset of spontaneous strokes are monitored which presents with unequivocal neurologic deficits followed by death within 24. This experimental design recapitulates the clinical scenario when a patient presents with acute onset of neurologic deficits due to a stroke.

### Materials and Methods

1. Single dose, IV-infusion of anti-DespR Mab-therapy was tested in spTg25 rats at onset of acute stroke signs, manifesting either as seizures, or limb paresis, or limb paralysis, or lethargy, or decorticate limb posture, or abnormal athetoid-like movements. Treatment was begun within 3 hours after detection by monitoring personnel and documentation of neurologic deficits by video. We note that detection by monitoring personnel does not coincide with actual start of neurologic deficits, but within 16 hours as all rats are monitored daily and found to be “ok” the day before stroke onset.

2. Controls comprised of littermate, genetically identical spTg25 rats that were mock-treated following the identical time table, with IV isotype antibody infusion using identical volumes and vehicle.

3. Observations after therapy or mock therapy were done hourly in the first 12 hours, then daily. Resolution of neurologic deficits in anti-DEspR Mab-treated rats was documented by video and observed to begin around 2-3 days, with almost complete recovery in 1 week (for rats that survived >1 week.)

4. No other therapies were given; no anti-hypertensive medications or anti-hypercholesterolemic medications were given. Rats were observed and euthanized upon signs of end-stage status. Treated rats did have recurrence of stroke after resolution of initial neurologic deficits, at different intervals. No further therapies were given.

### Results:

Effect of anti-DEspR treatment on stroke survival in Tg25 stroke-prone Dahl S rat model (Dahl S rats transgenic for human cholesteryl ester transfer protein).

Tg25 female rats were treated (IV infusion) with a single dose of either 10 μg of Isotype control (IgG1, n=10) or 10 μg of anti-DEspR 10A3H10 mAb (n=6) at stroke onset (rats were 4-6 months of age with documented neurological deficits). Rats were allowed to proceed to recovery up to eventual death. As shown in FIG. 29, a significant increase in post-stroke survival was observed upon anti-DEspR treatment (Mean post-stroke survival time for untreated controls=2.35±1.27 days versus Mean post-stroke survival time for anti-DEspR treated group=25.5±7.3 days; P=0.0007, Gehan-Breslow Test) extending post-stroke survival >ten-fold compared with littermate, genetically identical non-treated controls.

### Discussion

This experimental design simulates the stroking patient brought to the emergency room, and given anti-DEspR Mab therapy upon the detection of stroke signs (neurologic deficits). Just as would occur in patients, the time from onset of stroke to treatment in the emergency room would vary.

The spTg25 rat model is a model of human ischemic-hemorrhagic stroke, as well as a model of chronic low-flow ischemia leading to microhemorrhages (usually asymptomatic as observed in humans) and eventual macro/larger hemorrhages causing the presentation of neurologic deficits.

While some resolution of neurologic deficits is observed in patients due to the decrease of ensuing brain swelling from hypoxia/ischemia, it is clear from the observation of treated vs untreated rats that anti-DEspR treated rats have a more favorable survival outcome. Only 1/10 untreated rats had some resolution of neurologic deficits; all treated rats (6/6) had resolution of neurologic deficits and 5/6 lived longer than all untreated rats, and group mean survival from onset of 1st stroke is significantly different (P <0.0007).

We also note that resolution of neurologic deficits occurred in treated rats regardless of neurologic deficit: seizure, athetoid movement, lethargy, paresis, paralysis with decorticate positioning of limb.

Anti-DEspR Mab-therapy likely stabilizes the acute ischemic-hemorrhagic crisis presenting as neurologic deficits most likely due to the stabilization of leaky angiogenic microvessels thus decreasing further edema and hemorrhages. Leaky microvessels, just as observed in cancer, are most likely the product of angiogenesis induced by the chronic low flow ischemia present in this spTg25 rat model. Chronic low flow ischemia was previously documented in this model by MRI (Decano et al 2009). Blood pressure measurements in Dahl S female rats maintained in Harlan diet.

Five weeks old Dahl S female rats were implanted with radiotelemetry implants (PA-C10) and blood pressure was collected from 6-17 weeks of age. A single IV injection of 10 μg of 10A3H10 mAb was performed at 16 weeks of age with continuing blood pressure monitoring. Blood pressure was measured using intra-aortic abdominal radio-telemetric implants (PA-C10, Dataquest A.R.T. 4.2 system from DATA SCIENCES INTERNATIONAL) obtaining blood pressure measurements over ten-seconds every 5 minutes continuously from week 14-17. The average systolic (SBP), diastolic (DBP) and mean arterial pressures (MAP) were obtained, along with heart rate and activity.

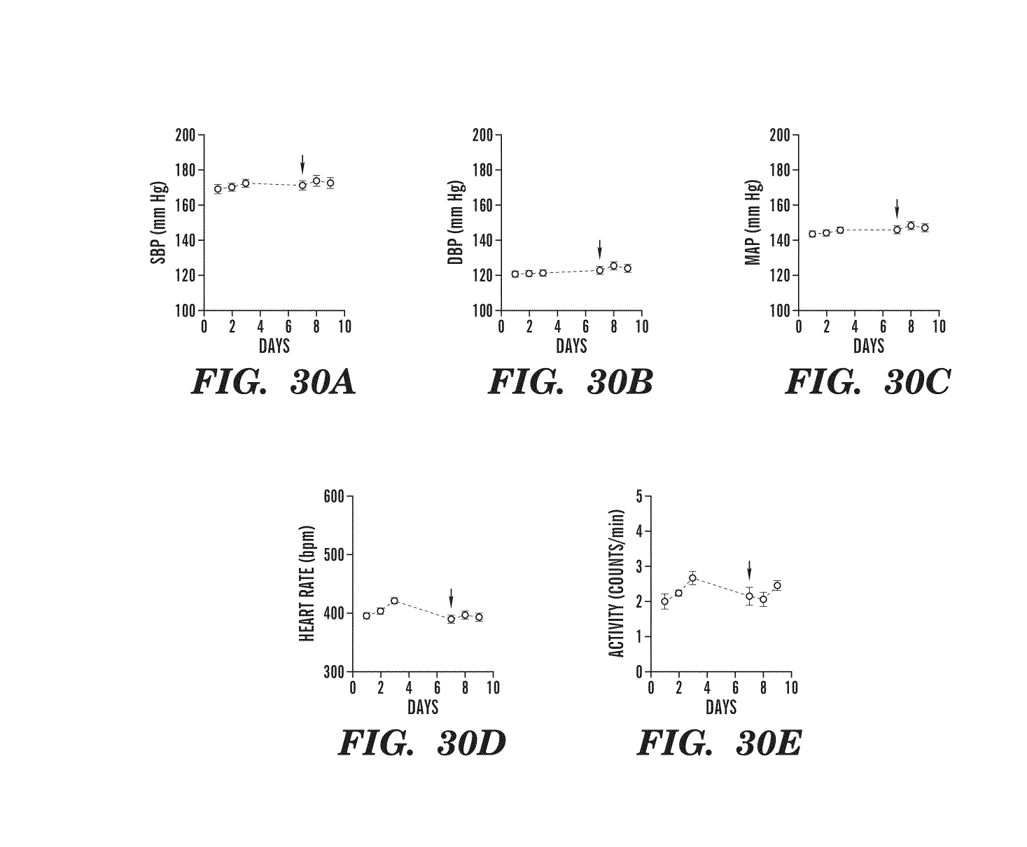

FIG. 30A-30E shows the analysis of blood pressure, heart rate and activity in Dahl S female rats challenge with 10A3H10 mAb. Dahl S female rats (n=6) at 16 weeks of age were injected with 10 μg of 10A3H10 mAb (IV). (30A) Mean systolic blood pressure ±sem (SBP; mmHg). (30B) Mean diastolic blood pressure ±sem (DBP; mmHg). (30C) Mean mean arterial pressure ±sem (MAP; mmHg). (30D) Mean heart rate ±sem (beats/min; bpm). (30E) Mean activity ±sem (Counts/min). Arrow indicates time of injection.

1. A method of treatment comprising
administering to a human subject within two days of the subject having a stroke a DEspR inhibitor in an amount effective to treat the stroke.

2-10. (canceled)

11. The method of claim 1, wherein the DEspR inhibitor comprises a human or humanized monoclonal antibody or fragment thereof that binds DEspR.

12-13. (canceled)

14. The method of claim 11, wherein the human or humanized monoclonal antibody or fragment thereof binds VEGFsp.

15-20. (canceled)

21. A method of inhibiting an adverse neurological event comprising,
administering to a human subject having or suspected of having micro-hemorrhages a DEspR inhibitor in an amount effective to inhibit the adverse neurological event.

22. The method of claim 21, wherein the adverse neurological event is further micro-hemorrhages.

23-24. (canceled)

25. A method of treating cancer comprising administering to a subject having a cancer expressing DEspR an antibody or fragment thereof that binds selectively to VEGFsp in an amount effective to inhibit the cancer.

26. (canceled)

27. The method of claim 25 wherein the antibody or fragment thereof is a human or humanized monoclonal antibody.

28-33. (canceled)

34. A method of inhibiting angiogenesis comprising administering to a subject having a disease or disorder dependent on or modulated by angiogenesis, an antibody or fragment thereof that binds selectively VEGFsp in an amount effective to inhibit the angiogenesis.

35. The method of claim 34 wherein the disease or disorder is age-related macular degeneration, carotid artery disease, diabetic retinopathy, rheumatoid arthritis, a neurodegenerative disease, Alzheimer's disease, obesity, endometriosis, psoriasis, atherosclerosis, ocular neovascularization, neovascular glaucoma, osteoporosis, or restenosis.

36. (canceled)

37. The method of claim 35 wherein the antibody or fragment thereof is a human or humanized monoclonal antibody.

38-43. (canceled)

44. A pharmaceutical preparation comprising a human or humanized antibody or fragment thereof that binds selectively VEGFsp and a pharmaceutically acceptable carrier constructed and arranged for administration to a human.

45. The pharmaceutical preparation of claim 44 wherein the antibody or fragment thereof is a monoclonal antibody.

46. The pharmaceutical preparation of claim 44 wherein the antibody or fragment thereof blocks binding of VEGFsp to DEspR.

47-52. (canceled)

53. A composition comprising VEGFsp, or a fragment thereof that binds DEspR, coupled to a toxin.

54. The composition of claim 53, wherein the VEGFsp or the fragment thereof that binds DEspR is covalently coupled to a toxin.

55-57. (canceled)

58. The composition of claim 53, wherein the VEGFsp or the fragment thereof that binds DEspR is coupled to a particle that is coupled to, coated with, embedded with or contains the toxin.

59-62. (canceled)

63. A pharmaceutical preparation comprising VEGFsp, or a fragment thereof that binds DEspR, coupled to a pharmaceutical agent, and a pharmaceutically acceptable carrier constructed and arranged for administration to a human.

64. The pharmaceutical preparation of claim 63, wherein the VEGFsp or the fragment thereof that binds DEspR is covalently coupled to a toxin.

65-67. (canceled)

68. The pharmaceutical preparation of claim 63, wherein the VEGFsp or the fragment thereof that binds DEspR is coupled to a particle that is coupled to, coated with, embedded with or contains the toxin.

69-72. (canceled)

73. A method for inhibiting growth of tumor cells comprising contacting tumor cells expressing DEspR with a DEspR agonist coupled to a toxin, in an amount effective to inhibit growth of the tumor.

74. The method of claim 73 wherein the tumor cells are in a subject who has had one or more of (i) radiation treatment for cancer, (ii) chemotherapy for cancer, or (iii) surgical treatment for cancer.

75. The method of claim 73 wherein the DEspR agonist is an antibody or fragment thereof that binds DEspR.

76. (canceled)

77. The method of claim 75 wherein the antibody or fragment thereof is a human or humanized monoclonal antibody.

78. (canceled)

79. The method of claim 73 wherein the DEspR agonist is VEGFsp or a fragment of VEGFsp that binds DEspR.

80. The method of claim 75, wherein the DEspR agonist is covalently coupled to a toxin.

81-83. (canceled)

84. The method of claim 80, wherein the DEspR agonist is coupled to a particle that is coupled to, coated with, embedded with or contains the toxin.

85-88. (canceled)

89. A method of reducing cancer re-occurrence comprising administering to a subject after the subject has had one or more of (i) radiation treatment for cancer, (ii) surgical treatment for cancer and (iii) chemotherapy treatment for cancer, a DEspR inhibitor in an amount effective to reduce cancer re-occurrence.

90. A method for identifying a circulating tumor cell comprising contacting a circulating tumor cell expressing DEspR with an agent that binds DEspR, and detecting the agent bound to the circulating tumor cell.

91. The method of claim 90, wherein the agent is (i) an antibody that binds DEspR or (ii) VEGFsp.

92. The method of claim 91, wherein the agent is labeled.


SEQUENCE LISTING (27 sequences)

SEQ ID NO: 1 - protein, 85 aa, Homo sapiens
MTMFKGSNEMKSRWNWGSITCIICFTCVGSQLSMSSSKASNFSGPLQLYQRELEIFIVLTDVPNYRLIKENSHLHTTIVDQGRTV

SEQ ID NO: 2 - protein, 26 aa, Homo sapiens
MNFLLSWVHWSLALLLYLHHAKWSQA

SEQ ID NO: 3 - DNA, 357 bp, Artificial Sequence
  source: /note=“Description of Artificial Sequence Synthetic polynucleotide”
caggtgcaactgaaggagtcaggacctggcctggtggcgccctcacagagcctgtccattacctgcactgtctctgggttctcattaaccagctatgatataagctggattcgccagccaccaggaaagggtctggagtggcttggagtaatatggactggtggaggcacaaattataattcagctttcatgtccagactgagcatcagcaaggacaactccaagagccaagttttcttaaaaatgaacagtctgcaaactgatgacacagccatatattactgtgtaagagatcgggattacgacgggtggtacttcgatgtctggggcgcagggaccacggtcaccgtctcctca

SEQ ID NO: 4 - protein, 119 aa, Artificial Sequence
  source: /note=“Description of Artificial Sequence Synthetic polypeptide”
QVQLKESGPGLVAPSQSLSITCTVSGFSLTSYDISWIRQPPGKGLEWLGVIWTGGGTNYNSAFMSRLSISKDNSKSQVFLKMNSLQTDDTAIYYCVRDRDYDGWYFDVWGAGTTVTVSS

SEQ ID NO: 5 - protein, 10 aa, Artificial Sequence
  source: /note=“Description of Artificial Sequence Synthetic peptide”
GFSLTSYDIS

SEQ ID NO: 6 - protein, 16 aa, Artificial Sequence
  source: /note=“Description of Artificial Sequence Synthetic peptide”
VIWTGGGTNYNSAFMS

SEQ ID NO: 7 - protein, 11 aa, Artificial Sequence
  source: /note=“Description of Artificial Sequence Synthetic peptide”
DRDYDGWYFDV

SEQ ID NO: 8 - DNA, 336 bp, Artificial Sequence
  source: /note=“Description of Artificial Sequence Synthetic polynucleotide”
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaa

SEQ ID NO: 9 - protein, 112 aa, Artificial Sequence
  source: /note=“Description of Artificial Sequence Synthetic polypeptide”
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 10 - protein, 16 aa, Artificial Sequence
  source: /note=“Description of Artificial Sequence Synthetic peptide”
RSSQSIVHSNGNTYLE

SEQ ID NO: 11 - protein, 7 aa, Artificial Sequence
  source: /note=“Description of Artificial Sequence Synthetic peptide”
KVSNRFS

SEQ ID NO: 12 - protein, 9 aa, Artificial Sequence
  source: /note=“Description of Artificial Sequence Synthetic peptide”
FQGSHVPYT

SEQ ID NO: 13 - protein, 119 aa, Artificial Sequence
  source: /note=“Description of Artificial Sequence Synthetic polypeptide”
QVQLQESGPGLVKPSQTLSLTCTVSGFSLTSYDISWIRQPPGKGLEWLGVIWTGGGTNYNSAFMSRLTISKDNSKSTVYLQMNSLRAEDTAIYYCVRDRDYDGWYFDVWGQGTTVTVSS

SEQ ID NO: 14 - protein, 119 aa, Artificial Sequence
  source: /note=“Description of Artificial Sequence Synthetic polypeptide”
QVQLQESGPGLVKPSQTLSLTCTVSGFSLTSYDISWIRQPPGKGLEWLGVIWTGGGTNYNSAFMSRLTISKDNSKNTVYLQMNSLRAEDTAIYYCVRDRDYDGWYFDVWGQGTTVTVSS

SEQ ID NO: 15 - protein, 119 aa, Artificial Sequence
  source: /note=“Description of Artificial Sequence Synthetic polypeptide”
QVQLQESGPGLVKPSQTLSLTCTVSGFSLTSYDISWIRQPPGKGLEWLGVIWTGGGTNYNSAFMSRFTISKDNSKNTVYLQMNSLRAEDTAIYYCVRDRDYDGWYFDVWGQGTTVTVSS

SEQ ID NO: 16 - protein, 119 aa, Artificial Sequence
  source: /note=“Description of Artificial Sequence Synthetic polypeptide”
QVQLQESGPGLVKPSQTLSLTCTVSGFSLTSYDISWIRQPPGKGLEWLGVIWTGGGTNYNSAFMSRLTISKDNSKNTVYLQMNSLRAEDTAVYYCVRDRDYDGWYFDVWGQGTTVTVSS

SEQ ID NO: 17 - protein, 119 aa, Artificial Sequence
  source: /note=“Description of Artificial Sequence Synthetic polypeptide”
QVQLQESGPGLVKPSQTLSLTCTVSGFSLTSYDISWIRQPPGKGLEWLGVIWTGGGTNYNSAFMSRFTISKDNSKNTVYLQMNSLRAEDTAVYYCVRDRDYDGWYFDVWGQGTTVTVSS

SEQ ID NO: 18 - protein, 112 aa, Artificial Sequence
  source: /note=“Description of Artificial Sequence Synthetic polypeptide”
DVLMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPYTFGQGTKLEIK

SEQ ID NO: 19 - protein, 112 aa, Artificial Sequence
  source: /note=“Description of Artificial Sequence Synthetic polypeptide”
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPYTFGQGTKLEIK

SEQ ID NO: 20 - protein, 9 aa, Homo sapiens
MTMFKGSNE

SEQ ID NO: 21 - DNA, 357 bp, Artificial Sequence
  source: /note=“Description of Artificial Sequence Synthetic polynucleotide”
caggtgcagctgcaggagagcggccctggcctggtgaagcctagccagaccctgagcctgacctgcaccgtgagcggcttcagcctgaccagctacgacatcagctggatcagacagcctcctggcaagggcctggagtggctgggcgtgatctggaccggcggcggcaccaactacaacagcgccttcatgagcagactgaccatcagcaaggacaacagcaagagcaccgtgtacctgcagatgaacagcctgagagccgaggacaccgccatctactactgcgtgagagacagagactacgacggctggtacttcgacgtgtggggccagggcaccaccgtgaccgtgagcagc

SEQ ID NO: 22 - DNA, 357 bp, Artificial Sequence
  source: /note=“Description of Artificial Sequence Synthetic polynucleotide”
caggtgcagctgcaggagagcggccctggcctggtgaagcctagccagaccctgagcctgacctgcaccgtgagcggcttcagcctgaccagctacgacatcagctggatcagacagcctcctggcaagggcctggagtggctgggcgtgatctggaccggcggcggcaccaactacaacagcgccttcatgagcagactgaccatcagcaaggacaacagcaagaacaccgtgtacctgcagatgaacagcctgagagccgaggacaccgccatctactactgcgtgagagacagagactacgacggctggtacttcgacgtgtggggccagggcaccaccgtgaccgtgagcagc

SEQ ID NO: 23 - DNA, 357 bp, Artificial Sequence
  source: /note=“Description of Artificial Sequence Synthetic polynucleotide”
caggtgcagctgcaggagagcggccctggcctggtgaagcctagccagaccctgagcctgacctgcaccgtgagcggcttcagcctgaccagctacgacatcagctggatcagacagcctcctggcaagggcctggagtggctgggcgtgatctggaccggcggcggcaccaactacaacagcgccttcatgagcagattcaccatcagcaaggacaacagcaagaacaccgtgtacctgcagatgaacagcctgagagccgaggacaccgccatctactactgcgtgagagacagagactacgacggctggtacttcgacgtgtggggccagggcaccaccgtgaccgtgagcagc

SEQ ID NO: 24 - DNA, 357 bp, Artificial Sequence
  source: /note=“Description of Artificial Sequence Synthetic polynucleotide”
caggtgcagctgcaggagagcggccctggcctggtgaagcctagccagaccctgagcctgacctgcaccgtgagcggcttcagcctgaccagctacgacatcagctggatcagacagcctcctggcaagggcctggagtggctgggcgtgatctggaccggcggcggcaccaactacaacagcgccttcatgagcagactgaccatcagcaaggacaacagcaagaacaccgtgtacctgcagatgaacagcctgagagccgaggacaccgccgtgtactactgcgtgagagacagagactacgacggctggtacttcgacgtgtggggccagggcaccaccgtgaccgtgagcagc

SEQ ID NO: 25 - DNA, 357 bp, Artificial Sequence
  source: /note=“Description of Artificial Sequence Synthetic polynucleotide”
caggtgcagctgcaggagagcggccctggcctggtgaagcctagccagaccctgagcctgacctgcaccgtgagcggcttcagcctgaccagctacgacatcagctggatcagacagcctcctggcaagggcctggagtggctgggcgtgatctggaccggcggcggcaccaactacaacagcgccttcatgagcagattcaccatcagcaaggacaacagcaagaacaccgtgtacctgcagatgaacagcctgagagccgaggacaccgccgtgtactactgcgtgagagacagagactacgacggctggtacttcgacgtgtggggccagggcaccaccgtgaccgtgagcagc

SEQ ID NO: 26 - DNA, 336 bp, Artificial Sequence
  source: /note=“Description of Artificial Sequence Synthetic polynucleotide”
gacgtgctgatgacccagagccctctgagcctgcctgtgaccctgggccagcctgccagcatcagctgcagaagcagccagagcatcgtgcacagcaacggcaacacctacctggagtggtacctgcagaagcctggccagagccctcagctgctgatctacaaggtgagcaacagattcagcggcgtgcctgacagattcagcggcagcggcagcggcaccgacttcaccctgaagatcagcagagtggaggccgaggacgtgggcgtgtactactgcttccagggcagccacgtgccttacaccttcggccagggcaccaagctggagatcaag

SEQ ID NO: 27 - DNA, 336 bp, Artificial Sequence
  source: /note=“Description of Artificial Sequence Synthetic polynucleotide”
gacgtggtgatgacccagagccctctgagcctgcctgtgaccctgggccagcctgccagcatcagctgcagaagcagccagagcatcgtgcacagcaacggcaacacctacctggagtggtacctgcagaagcctggccagagccctcagctgctgatctacaaggtgagcaacagattcagcggcgtgcctgacagattcagcggcagcggcagcggcaccgacttcaccctgaagatcagcagagtggaggccgaggacgtgggcgtgtactactgcttccagggcagccacgtgccttacaccttcggccagggcaccaagctggagatcaag